SEQUENCE LISTING (16 sequences)

SEQ ID NO: 1 - protein, 8 aa, Homo sapiens
DRVYHIPF

SEQ ID NO: 2 - protein, 13 aa, Homo sapiens
SYSMEHFRWGKPV

SEQ ID NO: 3 - protein, 14 aa, Homo sapiens
ALKRQGRTLYGFGG

SEQ ID NO: 4 - protein, 11 aa, Homo sapiens
RPKPQQFFGLM

SEQ ID NO: 5 - protein, 9 aa, Homo sapiens
HWSYGLRPG

SEQ ID NO: 6 - protein, 13 aa, synthetic construct
  LIPID 1: note = decapeptide lactone core resulting from the cyclization of the Thr-4 hydroxyl group onto the C-terminal carboxylate; note = N-terminal decanoyl fatty acid side chain
WNDTGKDADGSID

SEQ ID NO: 7 - protein, 36 aa, synthetic construct
  MOD_RES 1: note = The N-terminus is acylated; note = The C-terminus is a carboxamide
YTSLIHSLIEESQNQQEKNEQELLELDKWASLWNWF

SEQ ID NO: 8 - protein, 20 aa, Homo sapiens
  MOD_RES 1: note = Amino acid 1 is D-phenylalanine
FPRPGGGGNGDFEEIPEEYL

SEQ ID NO: 9 - DNA, 23 bp, synthetic construct
  source 1..23: mol_type = other DNA
  misc_feature 1: note = 12 carbon linker (5AmMC12) at the 5' end
tgcggttgtagtactcgtggccg

SEQ ID NO: 10 - DNA, 19 bp, synthetic construct
  source 1..19: mol_type = other DNA
  misc_feature 1: note = 12 carbon linker (5AmMC12) at the 5' end
ccctagttagccatctccc

SEQ ID NO: 11 - DNA, 19 bp, synthetic construct
  source 1..19: mol_type = other DNA
  misc_feature 19: note = 3'-labeled with Cy5
gggagatggctaactaggg

SEQ ID NO: 12 - DNA, 42 bp, synthetic construct
  source 1..42: mol_type = other DNA
  misc_feature 1: note = 12 carbon linker (5AmMC12) at the 5' end
gcagcggtgtgggggcagcggtgtgggggcagcggtgtgggg

SEQ ID NO: 13 - DNA, 42 bp, synthetic construct; identical to SEQ ID NO: 12
  source 1..42: mol_type = other DNA
  misc_feature 42: note = 3-AmMO label present

SEQ ID NO: 14 - DNA, 42 bp, synthetic construct; identical to SEQ ID NO: 12
  source 1..42: mol_type = other DNA

SEQ ID NO: 15 - DNA, 42 bp, synthetic construct
  source 1..42: mol_type = other DNA
ccccacaccgctgcccccacaccgctgcccccacaccgctgc

SEQ ID NO: 16 - DNA, 19 bp, synthetic construct
  source 1..19: mol_type = other DNA
ccctagttagccatctccc

(19) United States
(12) Patent Application Publication
(10) Pub. No.: US 2023/0068165 A1
(43) Pub. Date: Mar. 2, 2023

(54) PHOSPHINE-MEDIATED AMINE-AZIDE COUPLING IN IONIC LIQUID FOR BIOCONJUGATION REACTIONS

(71) Applicant: North Carolina State University, Raleigh, NC (US)

(72) Inventors: Jun Ohata, Raleigh, NC (US); Hisham El-Shaffey, Raleigh, NC (US); Elizabeth Jasmine Gross, Hillsborough, NC (US); Yvonne Hall, Raleigh, NC (US)

(21) Appl. No.: 17/877,083

(22) Filed: Jul. 29, 2022

Related U.S. Application Data
(60) Provisional application No. 63/328,592, filed on Apr. 7, 2022.
(60) Provisional application No. 63/227,155, filed on Jul. 29, 2021.

(51) Int. Cl. C07C 273/18

(52) U.S. Cl. CPC C07C 273/1809

(57) ABSTRACT

The use of ionic liquids as a solvent for chemoselective bioconjugation reactions is described. For example, methods of preparing bioconjugates in ionic liquids via a phosphine-mediated azide-amine reaction to form a urea linkage between a biomolecule substrate and a second molecule are described. Methods of preparing bioconjugates with amide or enamine linkages in ionic liquids are also described. The methods can be used to prepare tagged biomolecules, such as dye-tagged proteins, peptides, nucleic acids, or saccharides (e.g., aminosaccharides), for use in various applications; to form biomolecule-polymer conjugates; or to form biomolecule-therapeutic agent conjugates, such as antibody-drug conjugates.

### CROSS REFERENCE TO RELATED APPLICATION

The presently disclosed subject matter claims the benefit of and priority to U.S. Provisional Patent Application Ser. No. 63/328,592, filed Apr. 7, 2022; U.S. Provisional Patent Application Ser. No. 63/230,183, filed Aug. 6, 2021; and U.S. Provisional Patent Application Ser. No. 63/227,155, filed Jul. 29, 2021, the disclosures of each of which are incorporated herein by reference in their entireties.

### REFERENCE TO SEQUENCE LISTING XML SUBMITTED ELECTRONICALLY

The content of the Sequence Listing XML filed using Patent Center as an XML file (Name: 297-353-4.xml; Size: 19,000 bytes; and Date of Creation: Jul. 28, 2022) is incorporated herein by reference in its entirety.

### TECHNICAL FIELD

The subject matter described herein relates to chemoselective bioconjugation reactions (e.g. of proteins, nucleic acids, and saccharides) performed in ionic liquids. Exemplary bioconjugation reactions described herein include phosphine-mediated reactions between aminoalkyl groups and azides resulting in conjugates with urea linkages, diboron compound-mediated reactions between amino groups and carboxylic acids resulting in conjugates with amide linkages; and reactions between aminoalkyl groups and phosphonium aldehydes resulting in conjugates with enamine linkages.

### BACKGROUND

Bioconjugation technologies have become a cornerstone of multifaceted fields of chemistry and biology for various applications spanning therapeutics,^{1,2} enzyme activity profiling,³ elucidation of cellular processes,^{4,5} fluorescence imaging,⁶⁻⁸ and material preparation.⁹ Despite the wide array of available chemical tools that have provided for the rapid growth of such research fields, there remains a demand for development of additional chemical labeling methods, as individual experimental design can often benefit a particular technology for diverse aims.¹⁰ Traditional criteria of bioconjugation tool design includes method compatibility with aqueous media,¹¹⁻¹³ and a variety of approaches have been reported to overcome the difficulties of performing organic chemistry reactions in water.¹⁴⁻¹⁸ However, there is an ongoing need for methods that involve a novel medium for bioconjugation that is compatible with both biomolecules and organic chemistry reactions, as such a medium could provide access to traditionally unapproachable biomolecule labeling processes.^{19,20}

### Summary

This summary lists several embodiments of the presently disclosed subject matter, and in many cases lists variations and permutations of these embodiments. This summary is merely exemplary of the numerous and varied embodiments. Mention of one or more representative features of a given embodiment is likewise exemplary. Such an embodiment can typically exist with or without the feature(s) mentioned: likewise, those features can be applied to other embodiments of the presently disclosed subject matter, whether listed in this summary or not. To avoid excessive repetition, this Summary does not list or suggest all possible combinations of such features.

In some embodiments, the presently disclosed subject matter provides a method of performing a chemoselective bioconjugation reaction, the method comprising contacting a biomolecule substrate with a functionalized molecule, wherein said biomolecule substrate comprises one of the group comprising a peptide, a protein, and a nucleic acid, and wherein said functionalized molecule comprises at least one chemical functional group that can form a bond with a chemical functional group present in said biomolecule substrate, and wherein the contacting is performed in a reaction mixture comprising a solvent or solvent mixture comprising, consisting essentially of, or consisting of, an ionic liquid, thereby forming a bioconjugate product. In some embodiments, the biomolecule substrate comprises one of the group comprising an enzyme, an antigenic protein, a chemokine, a cytokine, a cellular receptor, a cellular receptor ligand, an aptamer, and an antibody or active fragment thereof.

In some embodiments, the biomolecule substrate comprises one or more aminoalkyl moiety, the functionalized molecule is an azide-containing compound: the reaction mixture further comprises a triarylphosphine; and the bioconjugation product comprises an urea linkage. In some embodiments, the aminoalkyl moiety comprises an amino group of a terminal amino acid residue in a protein or peptide or an amino group of a lysine residue side chain in a peptide or protein. In some embodiments, the azide-containing compound comprises an azide-containing derivative of one of the group consisting of a small molecule therapeutic agent; a nucleic acid; a lipid: a carbohydrate; a polymer; and a detectable label.

In some embodiments, the ionic liquid comprises one or more of the group comprising 1-butyl-1-methylpyrrolidinium trifluoromethanesulfonate (BMPy OTf), 1-butyl-1-methylpyrrolidinium bis(trifluoromethanesulfonyl)imide (BMPy NTf₂), 1-ethyl-3-methylimidazolium acetate (EMIM OAc), 1-butyl-3-methylimidazolium acetate (BMIM OAc), 1-butyl-3-methylimidazolium trifluoromethanesulfonate (BMIM OT), 1-butyl-3-methylimidazolium bis(trifluoromethanesulfonyl)imide (BMIM NTf₂), 1-butyl-3-methylimidazolium tetrafluoroborate (BMIM BF₄), and tributylethylphosphonium diethylphosphate (TBEP). In some embodiments, the contacting is performed at a temperature of about 20 degrees Celsius (° C.) to about 70° C. In some embodiments, the contacting is performed for about 30 minutes to about 72 hours.

In some embodiments, the biomolecule substrate is present in the reaction mixture at a concentration of about 0.025 millimolar (mM) to about 0.4 mM; the azide-containing compound is present at a concentration of about 3 mM to about 20 mM, and the triarylphosphine is present at a concentration of about 3 mM to about 7.5 mM. In some embodiments, the reaction mixture further comprises a bicarbonate buffer, a borate buffer, or an acetate buffer. In some embodiments, the reaction mixture comprises no more than 6% by volume water.

In some embodiments, the biomolecule substrate comprises a protein comprising one or more carboxylic acid group: the functionalized molecule comprises an amino group: the reaction mixture further comprises a diboron compound; and the bioconjugation product comprises an amide linkage.

In some embodiments, the biomolecule substrate comprises a protein comprising one or more aminoalkyl group; the functionalized molecule comprises a triarylphosphonium aldehyde; and the bioconjugation product comprises an enamine linkage.

In some embodiments, the presently disclosed subject matter provides a method of performing a chemoselective bioconjugation reaction, the method comprising contacting a first molecule and a second molecule in a reaction mixture comprising a triarylphosphine and a solvent or solvent mixture, wherein said first molecule comprises an aminoalkyl group, wherein said second molecule comprises an azide group, and wherein the solvent or solvent mixture comprises, consists essentially of, or consists of an ionic liquid, thereby forming a bioconjugate product comprising a urea linkage, and wherein at least one of said first molecule and said second molecule comprises a biomolecule or a derivative thereof, wherein said biomolecule or derivative thereof a biomolecule or derivative selected from the group consisting of a protein, a peptide, a nucleic acid, a carbohydrate, and derivatives thereof. In some embodiments, the solvent or solvent mixture comprises 6% by volume water or less.

In some embodiments, the first molecule is present in the reaction mixture at a concentration of about 0.025 millimolar (mM) to about 2 mM; the second molecule is present at a concentration of about 0.3 mM to about 125 mM, and the triarylphosphine is present at a concentration of about 3 mM to about 125 mM. In some embodiments, the reaction mixture further comprises a bicarbonate buffer, a borate buffer, or an acetate buffer. In some embodiments, at least one of said first molecule and said second molecule comprises a dye, a fluorophore, a polymer, an affinity label, a lipid, a small molecule therapeutic agent, and a radioisotope.

It is an object of the presently disclosed subject matter to provide methods of performing chemoselective bioconjugation reactions in ionic liquids. This and other objects are achieved in whole or in part by the presently disclosed subject matter. Further, an object of the presently disclosed subject matter having been stated above, other objects and advantages of the presently disclosed subject matter will become apparent to those skilled in the art after a study of the following description and Figures.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1E: Urea-forming amine-azide coupling reaction with triphenylphosphine (PPh₃) in ionic liquid. (FIG. 1A) Schematic diagram of general reaction scheme. (FIG. 1B) Schematic illustration of compatibility of ionic liquids with both biomolecules and organic reactions. BMPy OTf: Butylmethylpyrrolidinium trifluoromethanesulfonate (Butylmethylpyrrolidinium triflate). (FIG. 1C) Matrix-assisted laser desorption/ionization (MALDI)-MS analysis of reaction of angiotensin II peptide with azide 1 and PPh₃ in different media: aqueous (top), glycerol (middle), and BMPy OTf (bottom). (FIG. 1D) NMR analysis of ¹⁵N-enriched urea product. Nitrogen atoms with “*” contain 1:1 ratio of ¹⁴N and ¹⁵N derived from parent ¹⁵N-enriched sodium azide.³² (FIG. 1E) Ultraviolet (UV) chromatograms (280 nm) of the liquid chromatography-mass spectrometry (LC-MS) analysis of crude reaction mixtures of daptomycin modification (without alkali hydrolysis) with azide 2 in ionic liquid in the presence of different amounts of water. Prior to the work-up process of the reaction mixtures (i.e. acetone precipitation), total volume of ionic liquid in each condition was adjusted to be the same (i.e. an additional 4%, 19%, or 39% BMPy OTf was introduced to 5%, 20%, and 40% water conditions, respectively) to eliminate bias during the workup.

FIGS. 2A-2C: Urea formation reaction on peptide substrates. Reaction conditions: peptide (0.1-0.4 mM), KHCO₃ (20 mM), azide (7.5 mM), PPh₃ (20 mM) in BMPy OTf at 50° C. for 2 h. *4 h reaction time. (FIG. 2A) Chemical structure and amino acid sequence of peptide substrates: angiotensin II (SEQ ID NO:1), alpha-melanocyte-stimulating hormone (α-MSH; SEQ ID NO:2), osteogenic growth peptide (OGP: SEQ ID NO:3), substance P (SEQ ID NO:4), luteinizing hormone-releasing hormone (LHRH; SEQ ID NO:5), and daptomycin (SEQ ID NO:6). Alkylamine-containing amino acid residue highlighted in red. Conversion in the parentheses and the modification ratio were obtained by the peak area integration in liquid-chromatography (LC) analysis. (FIG. 2B) Tandem mass spectrometry analysis of angiotensin II (SEQ ID NO: 1). Observed y and b ions are highlighted in green and cyan, respectively. (FIG. 2C) LC-mass spectrometry (LC-MS) analysis of modification of alkali-hydrolyzed daptomycin by azide 2 with PPh₃ or O═PPh₃. SD: Internal standard for the LC experiment (benzoyl-arginine ethyl ester).

FIGS. 3A-3E: Urea formation reaction on proteins with conservation of native protein activity. Typical modification conditions: proteins (0.025-0.075 mM), KHCO₃ (20 mM), alkyl azide (3-7.5 mM), PPh₃ (3-7.5 mM) in BMPyOTf at 37° C. for 2 h. Error bars represent standard deviation (n=3). (FIG. 3A) Schematic drawing of structures of proteins modified by azide reagents: enfuvirtide, lysozyme, α-chymotrypsinogen A, and streptavidin. Conversion was calculated based on the intensity in the mass spectrometry analysis. (FIG. 3B) Plot of fluorescence intensity of anti-biotin western blot for modification of α-chymotrypsinogen A with biotin azide at different time points (0 to 40 minutes (min)). (FIG. 3C) Structure of cyanine-based azide (3″, Cy3-azide) and gel fluorescence analysis of modification of α-chymotrypsinogen A with 3″. (FIG. 3D) Bar graph representing chemiluminescence intensity of anti-biotin western blot of biotinylated α-chymotrypsinogen A with the urea linker incubated in the designated condition. MES: (N-morpholino)ethanesulfonic acid (50 mM). GSH: glutathione. HEPES: 2-[4-(2-hydroxyethyl)piperazin-1-yl]ethanesulfonic acid (50 mM). (FIG. 3E) Bar graph representing fluorescence intensity of streptavidin incubated with biotin-fluorescein after the urea-forming reaction in the designated buffer as well as the buffer exchange.

FIGS. 4A-4C: Urea-forming bioconjugation on antibodies. Modification conditions: antibody (0.01 mM), KHCO₃ (20 mM), alkylazide (3 mM), PPh₃ (3 mM) in BMPyOTf at 37° C. for 2 h. (FIG. 4A) Structure of antibody conjugates with biotin and isotopoisomerase inhibitor (SN-38). (FIG. 4B) Blot analysis of Trastuzumab (sold under the tradename HERCEPTIN®) modification with biotin-azide in the presence of triphenylphosphine (circle) or triphenylphosphine oxide (rectangle). The reactions were analyzed by anti-biotin western blot with streptavidin-fluorophore (Cy5) conjugate. Reduction (lane #iii and iv) was performed with tris(2-carboxyethyl)phosphine (TCEP, 5 mM) at rt for 5 min. Total stain: Ponceau S stain of the blot membrane. HC and LC: heavy and light chains, respectively. (FIG. 4C) Confocal microscope images of SK-BR-3. Her2-overexpressing cell line stained with unmodified (top) or SN-38-modified (bottom) Trastuzumab, visualized by anti-human secondary antibody-fluorophore (Cy5) conjugate (red). Blue: Nuclear stain with DAPI. DIC: Differential interference contrast. Scale bar: 20 μm.

FIG. 5: Schematic drawing of a diboron-catalyzed bioconjugation reaction between a carboxylic acid moiety of a biomolecule substrate (e.g., a protein) and an amino moiety of a functionalized molecule performed in an ionic liquid. The bioconjugation reaction forms a bioconjugate comprising an amide linkage.

FIG. 6: Schematic drawing of a bioconjugation reaction between an amino group on a protein and a triarylphosphonium aldehyde performed in an ionic liquid to form an enamine linkage.

FIG. 7: Schematic drawing of a site-specific phosphine-mediated urea-forming azide-amino reaction with DNA substrates performed in ionic liquids.

FIGS. 8A-8E: Alkylamine-selective urea-forming reaction with pentanucleotides in an ionic liquid. Reaction conditions: KHCO₃ (20 mM), XTTTT (0.2 mM), azide 1a (7.5 mM), and PPh₃ (20 mM) in 1-butyl-1-methylpyrrolidinium trifluoromethanesulfonate (BMPy OTf) at 50° C. for 2 h. (FIG. 8A) Chemical structure of nucleotide backbone in an XTTTT sequence with or without an alkylamine. (FIG. 8B) Chemical structure of azide 1a. (FIG. 8C) Structure of the ionic liquid, BMPy OTf. (FIGS. 8D and 8E) Matrix-assisted laser desorption/ionization (MALDI-MS) analysis of the reaction of XTTIT where X=adenosine (FIG. 8D, top), thymidine (FIG. 8D, bottom), cytidine (FIG. 8E, top), guanosine (FIG. 8E, second from top), deoxyuridine (FIG. 8E, second from bottom), and thymidine with alkylamine containing a 12 carbon linker (5AmMC12) (FIG. 8E, bottom).

FIG. 9: Urea-forming DNA bioconjugation with a variety of alkylazides. Modification reaction conditions: KHCO₃ (20 mM), 5′-TTTTT-3′-alkyl-NH₂ (0.2 mM), azide derivatives 1b-1j (7.5 mM), and PPh₃ (20 mM) in BMPy OTf at 50° C. for 2 h. *Reaction was incubated overnight. Conversion in the parentheses were calculated based on matrix-assisted laser desorption/ionization (MALDI-MS) analysis.

FIGS. 10A-10D: Screening of phosphine reagents in urea formation reaction on a DNA aptamer toward human serum albumin (HSA). Reaction conditions: Aminoalkyl-modified HSA DNA aptamer (HSA aptamer-5′-NH₂ (SEQ ID NO: 9), 0.1 mM), KHCO₃ (20 mM), biotin azide (7.5 mM), and phosphine (3.0 mM) in a mixture of 1-ethyl-3-methylimidazolium acetate (EMIM OAc)/DMF (1:1) at 50° C. for 2 h. (FIG. 10A) General chemical structure of phosphine reagents. The circle, square and triangle represent the aryl or alkyl groups shown in FIG. 10C. (FIG. 10B) Chemical structure of ionic liquid EMIM OAc. (FIG. 10C) Chemical structures of the aryl or alkyl groups of different phosphine/phosphite reagents. (FIG. 10D) Bar graph showing the anti-biotin southern blot after modification of the HSA DNA aptamer with biotin azide with different phosphines. Error bars represent standard deviation (n=3). Representative blot membrane images for the anti-biotin southern blot (Cy5) and total stain with Mayer's hemalum solution (MHS) are shown below the bar graphs.

FIGS. 11A-11C: Reactivity and selectivity analysis of the urea forming reaction on pentanucleotides with an alkylamine in different locations. (FIG. 11A) Bar graph showing fluorescence intensity of 5′-TTTTT-3′ with and without an alkyl-amine and treated with fluorophore azide 1k. Reaction conditions; KHCO₃ (20 mM), 5′-TTTTT-3′ with and without amine group (0.2 mM), azide 1k (3 mM), and PPh₃ (3 mM) in BMPy OTf at 50° C. for a specific time. Error bars represent standard deviation (n=3). (FIG. 11B) Chemical structure of alkylazide containing a boron dipyrromethene (BODIPY) group (1k). (FIG. 11C) Agarose gel images for the reaction of TTTTT-5′-NH₂ with azide 1k in the presence of DNA ladder (10-300 bps). Total DNA samples were visualized by the fluorescence from SYBR® Gold nucleic acid gel stain (Cy3 excitation and emission), while modified DNA samples were visualized by the fluorescence from BODIPY (Cy2 excitation and emission). Reaction conditions: KHCO₃ (20 mM), oligonucleotide with or without NH₂ tag at 5′ position (0.2 mM), DNA mixture (0.9 mg/mL), azide 1k (3 mM), and PPh₃ (3 mM) in BMPy OTf at 50° C. for 2 h. Chemical structures of the alkylamine groups on different positions (internal, 3′, and 5′) are shown in Scheme 1 in the Examples.

FIGS. 12A-12D: Attachment of cholesterol to a DNA aptamer toward the RNA hairpin of human immunovirus (HIV)-1 transactivation-responsive (TAR) element through the urea-forming reaction. Sequence of the HIV-1-TAR aptamer: 5′-CCCTAGTTAGCCATCTCCC-3′ (SEQ ID NO: 16).⁹⁸ Modification conditions: HIV-1-TAR-5′-NH₂ aptamer (SEQ ID NO: 10; 0.1 mM), KHCO₃ (20 mM), azide 11 (7.5 mM), and PPh₃ (2) or O═PPh₃ (3) (20 mM) in EMIM OAc/DMF/DMSO (2:1:1) at 50° C. for 2 h. (FIG. 12A) Chemical structure of cholesterol azide (01). (FIG. 12B) Agarose gel analysis of HIV-1-TAR-5′-NH₂ aptamer (SEQ ID NO: 10) modified with cholesterol azide (1l) in the presence of triphenylphosphine oxide (3), triphenylphosphine (2a) JohnPhos (2h), and sulfonate-substituted triphenylphosphine (2e). Degree of conversion was calculated by quantification of the unmodified DNA bands in comparison with the triphenylphosphine oxide condition as 100% of the starting material. (FIG. 12C) Matrix-assisted laser desorption/ionization (MALDI-MS) analysis of the modification of HIV-1-TAR-5′-NH₂ aptamer (SEQ ID NO: 10) with azide 11 and triphenylphosphine oxide (top, negative control) or triphenylphosphine (bottom). (FIG. 12D) Confocal microscopy images of HeLa cells stained with HIV-TAR aptamer (SEQ ID NO: 10) hybridized with its complementary DNA-Cy5 conjugate (magenta). Top: Cholesterol-modified aptamer (azide/PPh₃-treated aptamer). Bottom: Unmodified aptamer (azide/O═PPh₃-treated aptamer). Green: Actin filament stain with Phalloidin-CF488 conjugate. Scale bar: 20 μm.

FIGS. 13A and 13B: Urea-forming reaction of DNA aptamer with the Her2 receptor. Sequence of the Her2 aptamer: 5′-GCAGCGGTGTGGGGGCAGCGGTGTGGGGGCAGCGGTGTGGGG-3 (SEQ ID NO: 14)¹⁰² Modification conditions: Her2 aptamer-5′-NH₂ aptamer (SEQ ID NO: 12, 0.1 mM), KHCO₃ (20 mM), biotin azide (7.5 mM), and PPh₃ (20 mM) in EMIM OAc/DMF/DMSO (2:1:1 ratio) at 50° C. for 2 h. (FIG. 13A) Partial crystal structure of Her2 receptor (PDB ID: 1N8Z). (FIG. 13B) Confocal microscopy images of Her2-overexpressing SK-BR-3 cells stained with biotin-modified Her2 aptamer (top), unmodified aptamer (middle), or biotin-modified Her2 aptamer hybridized with complementary DNA sequence (bottom). The bound aptamer was visualized by streptavidin-fluorophore (Cy5) conjugate (1:50 dilution) shown in magenta. Blue: Nuclear stain with DAPI. DIC: differential interference contrast. Scale bar: 20 μm.

FIG. 14: Schematic drawing of site-specific bioconjugation reactions on carbohydrate compounds. Hexagon structures indicate carbohydrate, while the star represents functionality such as visualization, affinity, and reactive handles.

FIGS. 15A-15D: Characterization of the urea structure formed in the phosphine-mediated amine-azide coupling reaction of a carbohydrate substrate. (FIG. 15A) A schematic diagram for the urea-forming reaction. The “*” sign denotes the ¹⁵N-labeling. (FIG. 15B) X-ray structure of the reaction product of aminomethyl-pyrene and azidomethyl-pyrene at 60% probability level. Hydrogen atoms on the pyrene rings are omitted for clarity. Shaded red ellipsoid: oxygen. Shaded blue ellipsoid: nitrogen. Black ellipsoid: carbon. Plain sphere: hydrogen. Selected bond lengths (Å) and angles (deg): N1-C1 1.355(2), N2-C1 1.355(2), C1-O1 1.237(4), N1-C1-N2 115.6(3), N1-C1-O1 122.21(13), N2-C1-O1 122.21(13). (FIG. 15C) Electrospray ionization mass spectrometry (ESI-MS) analysis of the modification of amine-containing molecule daptomycin under air, ¹²CO₂, or ¹³CO₂. (FIG. 15D) ¹³C {¹H} NMR spectra of urea products with and without isotope labels. “*” sign in middle spectra denotes 50% ¹⁵N incorporation and in the spectra on the right denotes its 100% incorporation.

FIGS. 16A-16C: Post-reaction clean-up processes of ionic liquid-based carbohydrate bioconjugation prior to downstream analysis. IL: ionic liquid. Aq. sol.: aqueous solution. Org. solv.: organic solvent. (FIG. 16A) Schematic diagrams and photographic images for liquid-liquid extraction and precipitation of hydrophilic saccharides. (1, top)(Left) schematic diagram of extraction processes for water-soluble saccharides. (Right) An image of extraction process of fluorescein isothiocyanate (FITC)-labeled dextran from a mixture of butylmethylpyrrolidinium triflate (BMPy OTf)/bistriflimide (BMPy NTf₂) by using 1× phosphate-buffered saline. (2, bottom) (Left) Schematic diagram of precipitation process with acetone and (right) a photographic image of pellets of FITC-labeled vancomycin from a BMPy OTf reaction mixture. (FIG. 16B) A general scheme (left) and photographic image (right) of extraction of hydrophobic carbohydrate valrubicin from ethylmethylimidazolium acetate (EMIM OAc) reaction mixture using ethyl acetate. (FIG. 16C) Schematic diagram of thin layer chromatography (TLC)-based purification of a hydrophobic saccharide. A mixture of hydrophobic saccharide valrubicin (orange) with model compounds Coomassie Brilliant Blue (blue) and amine-containing nitrobenzofurazan (green) was subjected to reverse-phase TLC, followed by extraction of the target compound from stationary phase with hexafluoroisopropanol (the photographic image of the orange solution on the right).

FIGS. 17A-17E: Triarylphosphine-mediated bioconjugation of small saccharides in ionic liquid. Reaction conditions: KHCO₃ (20 mM), saccharides (0.3-0.5 mM), azide 2 or S4 (7.5-125 mM), PAr₃ (3-125 mM) in butylmethylpyrrolidinium triflate (BMPy OTf) at 37-50° C. for 2 h. (FIG. 17A) General reaction scheme targeting primary and secondary alkylamine groups. (FIG. 17B) Structures of amine-containing anthracene derivatives (2a′-2d′) and bar graph of percentage conversion of their modification with azide 2. Error bars represent standard deviation (n=3). (FIG. 17C) Chemical structure of doxorubicin (3a), a primary amine containing antitumor agent. Chemical structure of valrubicin (3b) bearing a capped amine. (FIG. 17D) Liquid chromatography-mass spectrometry (LC-MS) analysis of reactions of doxorubicin (3a, left) and valrubicin (3b, right) with and without PPh₂Ar (Ar=m-sulfophenyl). (FIG. 17E) Chemical structure of vancomycin and derivatives 4a-4c and LC analysis of the modification reaction with azide S4. The different substituents are highlighted in red and magenta while the monomethyl secondary amine is highlighted in green. The modification ratio and/or conversion were obtained by peak intensity (anthracene derivatives) or peak area integration (doxorubicin and vancomycin derivatives) in UV chromatograms.

FIGS. 18A-18E: The phosphine-mediated reaction on chitosan amine-containing polysaccharide. Typical modification conditions. Chitosan or diethylaminoethyl (DEAE)-dextran (1 mg/mL), biotin-azide (3 mM), and PPh₃ (3 mM) in ethylmethylimidazolium acetate (EMIM OAc) at 37° C. for 2 h. Error bars represent standard deviation (n=3). (FIG. 18A) Chemical structure of chitosan (5a) and DEAE-dextran (5b). (FIG. 18B) Bar graph showing the fluorescence intensity from anti-biotin blot for modification of chitosan and DEAE-dextran with biotin-azide. (FIG. 18C) A plot of fluorescence intensity of anti-biotin blot for modification of chitosan with different concentrations of biotin-azide (0, 0.3, 0.75, 1.5, and 3 mM) and triphenylphosphine (0, 0.3, 0.75, 1.5, and 3 mM). (FIG. 18D) A bar graph showing the fluorescence intensity of anti-biotin blot for labeling chitosan in a variety of ionic liquids. Control: A negative control experiment with O═PPh₃ in EMIM OAc instead of PPh₃. (FIG. 18E) Chemical structure of ionic liquids: BMPy: butylpyridinium. BMIM: butylmethylimidazolium. EMIM: ethylmethylimidazolium. OTf: triflate. OAc: acetate. BF₄: tetrafluoroborate. NTf₂: bistriflimide.

FIGS. 19A-19G. Phosphine-mediated bioconjugation of azide containing saccharides in ionic liquid using excess amine reagents. Reaction conditions: azide-containing saccharide (0.1 mM or 1 mg/mL), KHCO₃ (20 mM), amine reagent (3-20 mM), and PPh₃ (20 mM) in butylpyrrolium triflate (BMPy OTf) or ethylmethylimidazolium acetate (EMIM OAc) at 37° C. Error bars represent standard deviation (n=3). (FIG. 19A) General reaction scheme. (FIG. 19B) ¹H NMR analysis of the reaction of an azide substrate modified with excess alkylamine reagent (bottom), compared with the reaction of an aminoalkyl group of an amino acid substrate reacting with excess alkylazide reagent (top). (FIG. 19C) Matrix-assisted laser desorption/ionization mass spectrometry (MALDI-MS) analysis of the reaction of azide-containing nonasaccharide with benzyl amine derivatives 6a-6c and PPh₃ (top row) or O═PPh₃ (bottom row). “*” indicates potassium adducts. (FIG. 19D) Chemical structure of hyaluronic acid with and without the azide tag (left), chemical structure of fluorophore-alkylamine reagent (6d, middle), and bar graph of quantification of dot blot analysis of the hyaluronic acid modification with the fluorophore amine (right). (FIG. 19E) Modification of hyaluronic acid derivatives with amine reagents containing alkyne (6e, left and 6f, middle) and trans-cyclooctene (6g, right), which were subject to the secondary modification with fluorophore bearing azide and tetrazine, respectively. Bar graphs represent quantification of dot blot analysis of the modification reaction. (FIG. 19F) Chemical structure of amine-functionalized biotin (6h) used in the cell lysate study. (FIG. 19G) Anti-biotin western blot and total stain (Ponceau S.) of the biotinylating reaction of human embryo kidney (HEK) 293T cells in BMPy OTf with and without the azide tag.

### DETAILED DESCRIPTION

The presently disclosed subject matter will now be described more fully. The presently disclosed subject matter can, however, be embodied in different forms and should not be construed as limited to the embodiments set forth herein below and in the accompanying Examples. Rather, these embodiments are provided so that this disclosure will be thorough and complete, and will fully convey the scope of the embodiments to those skilled in the art.

All references listed herein, including but not limited to all patents, patent applications and publications thereof, and scientific journal articles, are incorporated herein by reference in their entireties to the extent that they supplement, explain, provide a background for, or teach methodology, techniques, and/or compositions employed herein.

### I. Definitions

While the following terms are believed to be well understood by one of ordinary skill in the art, the following definitions are set forth to facilitate explanation of the presently claimed subject matter.

Following long-standing patent law convention, the terms “a”, “an”, and “the” refer to “one or more” when used herein, including in the claims.

As used herein, the term “about”, when referring to a value or an amount, for example, relative to another measure, is meant to encompass variations of in some embodiments ±20%, in some embodiments ±10%, in some embodiments ±5%, in some embodiments ±1%, and in some embodiments ±0.1% from the specified value or amount, as such variations are appropriate. The term “about” can be applied to all values set forth herein.

As used herein, ranges can be expressed as from “about” one particular value, and/or to “about” another particular value. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, the term “and/or” when used in the context of a listing of entities, refers to the entities being present singly or in combination. Thus, for example, the phrase “A, B, C, and/or D” includes A, B, C, and D individually, but also includes any and all combinations and sub-combinations of A, B, C, and D.

The term “comprising”, which is synonymous with “including,” “containing,” or “characterized by” is inclusive or open-ended and does not exclude additional, unrecited elements or method steps. “Comprising” is a term of art used in claim language which means that the named elements are present, but other elements can be added and still form a construct or method within the scope of the claim.

As used herein, the phrase “consisting of” excludes any element, step, or ingredient not specified in the claim. When the phrase “consists of” appears in a clause of the body of a claim, rather than immediately following the preamble, it limits only the element set forth in that clause: other elements are not excluded from the claim as a whole.

As used herein, the phrase “consisting essentially of” limits the scope of a claim to the specified materials or steps, plus those that do not materially affect the basic and novel characteristic(s) of the claimed subject matter.

With respect to the terms “comprising”, “consisting of”, and “consisting essentially of”, where one of these three terms is used herein, the presently disclosed and claimed subject matter can include the use of either of the other two terms.

As used herein, amino acids are represented by the full name thereof, by the three letter code corresponding thereto, or by the one-letter code corresponding thereto, as indicated in Table 1:

TABLE 1
Amino Acid Codes and Functionally
Equivalent Codons

| | 3- | 1- | |
|---|---|---|---|
| | Letter | Letter | Functionally |
| Full Name | Code | Code | Equivalent Codons |
| Aspartic Acid | Asp | D | GAC; GAU |
| Glutamic | Glu | E | GAA; GAG |
| Acid | | | |
| Lysine | Lys | K | AAA; AAG |
| Arginine | Arg | R | AGA; AGG; CGA; CGC; |
| | | | CGG; CGU |
| Histidine | His | H | CAC; CAU |
| Tyrosine | Tyr | Y | UAC; UAU |
| Cysteine | Cys | C | UGC; UGU |
| Asparagine | Asn | N | AAC; AAU |
| Glutamine | Gln | Q | CAA; CAG |
| Serine | Ser | S | ACG; AGU; UCA; UCC; |
| | | | UCG; UCU |
| Threonine | Thr | T | ACA; ACC; ACG; ACU |
| Glycine | Gly | G | GGA; GGC; GGG; GGU |
| Alanine | Ala | A | GCA; GCC; GCG; GCU |
| Valine | Val | V | GUA; GUC; GUG; GUU |
| Leucine | Leu | L | UUA; UUG; CUA; CUC; |
| | | | CUG; CUU |
| Isoleucine | Ile | I | AUA; AUC; AUU |
| Methionine | Met | M | AUG |
| Proline | Pro | P | CCA; CCC; CCG; CCU |
| Phenylalanine | Phe | F | UUC; UUU |
| Tryptophan | Trp | W | UGG |

The expression “amino acid” as used herein is meant to include both natural and synthetic amino acids, and both D and L amino acids. “Standard amino acid” means any of the twenty standard L-amino acids commonly found in naturally occurring peptides. “Nonstandard amino acid residue” means any amino acid, other than the standard amino acids, regardless of whether it is prepared synthetically or derived from a natural source. As used herein, “synthetic amino acid” also encompasses chemically modified amino acids, including but not limited to salts, amino acid derivatives (such as amides), and substitutions. Amino acids contained within the peptides of the presently disclosed subject matter, and particularly at the carboxy- or amino-terminus, can be modified by methylation, amidation, acetylation or substitution with other chemical groups which can change the peptide's circulating half-life without adversely affecting their activity. Additionally, a disulfide linkage can be present or absent in the peptides of the presently disclosed subject matter.

The term “amino acid” is used interchangeably with “amino acid residue,” and can refer to a free amino acid or to an amino acid residue of a peptide. It will be apparent from the context in which the term is used whether it refers to a free amino acid or a residue of a peptide or protein.

Amino acids can be classified into seven groups on the basis of the side chain R: (1) aliphatic side chains, (2) side chains containing a hydroxylic (OH) group, (3) side chains containing sulfur atoms, (4) side chains containing an acidic or amide group, (5) side chains containing a basic group. (6) side chains containing an aromatic ring, and (7) proline, an imino acid in which the side chain is fused to the amino group.

Amino acids have the following general structure:

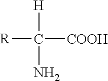

The nomenclature used to describe the peptide compounds of the presently disclosed subject matter follows the conventional practice wherein the amino group is presented to the left and the carboxy group to the right of each amino acid residue. In the formulae representing selected specific embodiments of the presently disclosed subject matter, the amino- and carboxy-terminal groups, although not specifically shown, will be understood to be in the form they would assume at physiologic pH values, unless otherwise specified.

The term “basic” or “positively charged” amino acid, as used herein, refers to amino acids in which the R groups have a net positive charge at pH 7.0, and include, but are not limited to, the standard amino acids lysine, arginine, and histidine.

As used herein, the term “conservative amino acid substitution” is defined herein as an amino acid exchange within one of the five groups summarized in the following Table 2.

TABLE 2
Conservative Amino Acid Substitutions

| Group | Characteristics | Amino Acids |
|---|---|---|
| A. | Small aliphatic, nonpolar or slightly | Ala, Ser, Thr, Pro, |
| | polar residues | Gly |
| B. | Polar, negatively charged residues | Asp, Asn, Glu, Gln |
| | and their amides | |
| C. | Polar, positively charged residues | His, Arg, Lys |
| D. | Large, aliphatic, nonpolar residues | Met Leu, Ile, Val, |
| | | Cys |
| E. | Large, aromatic residues | Phe, Tyr, Trp |

The terms “amino-containing saccharide” and “aminosaccharide” as used herein refer to molecules comprising one or more saccharide group having an amino substituent (i.e., one or more “aminosugar”). Thus, the term “aminosaccharide” refers to any synthetic or naturally occurring saccharide wherein one or more carbon atoms are substituted with an amino group (e.g., NH₂). Such substitution can occur without regard to orientation or configuration of any asymmetric carbons present in the saccharide. Unless stated otherwise, the term “aminosugar” refers to either anomer (α or β) of a cyclic aminosaccharide. Aminosugars can be N-substituted with alkyl or acyl group, w % here one hydrogen atom of a pendant amino group is replaced by an alkyl or acyl moiety (e.g., C(═O)R where R is lower alkyl, such as methyl)). Representative aminosugars include, but are not limited to, L-vancosamine, 3-desmethyl-vancosamine, 3-epi-vancosamine, 4-epi-vancosamine, acosamine, actinosamine, daunosamine, 3-epi-daunosamine, ristosamine, N-methyl-D-glucamine, N-acetylglucosamine, glucosamine, galactosamine, N-acetylgalactosamine, iminocyclitol, and the like.

As used herein, an “analog” of a chemical compound is a compound that, by way of example, resembles another in structure but is not necessarily an isomer (e.g., 5-fluorouracil is an analog of thymine).

The term “antibody”, as used herein, refers to an immunoglobulin molecule which is able to specifically or selectively bind to a specific epitope on an antigen. Antibodies can be intact immunoglobulins derived from natural sources or from recombinant sources and can be immunoreactive portions of intact immunoglobulins. Antibodies are typically tetramers of immunoglobulin molecules. The antibodies in the presently disclosed subject matter can exist in a variety of forms. The term “antibody” refers to polyclonal and monoclonal antibodies and derivatives thereof (including chimeric, synthesized, humanized and human antibodies), including an entire immunoglobulin or antibody or any functional fragment of an immunoglobulin molecule which binds to the target antigen and or combinations thereof. Examples of such functional entities include complete antibody molecules, antibody fragments, such as Fᵥ, single chain Fᵥ, complementarity determining regions (CDRs), V_{L} (light chain variable region), V_{H} (heavy chain variable region), Fab, F(ab′)₂ and any combination of those or any other functional portion of an immunoglobulin peptide capable of binding to target antigen.

Antibodies exist, e.g., as intact immunoglobulins or as a number of well characterized fragments produced by digestion with various peptidases. Thus, for example, pepsin digests an antibody below the disulfide linkages in the hinge region to produce F(ab′)₂ a dimer of Fab which itself is a light chain joined to V_{H}-C_{H1} by a disulfide bond. The F(ab′)₂ can be reduced under mild conditions to break the disulfide linkage in the hinge region, thereby converting the F(ab′)₂ dimer into an Fab₁ monomer. The Fab₁ monomer is essentially a Fab with part of the hinge region (see Paul, 1993). While various antibody fragments are defined in terms of the digestion of an intact antibody, one of skill will appreciate that such fragments can be synthesized de now) either chemically or by utilizing recombinant DNA methodology. Thus, the term antibody, as used herein, also includes antibody fragments either produced by the modification of whole antibodies or those synthesized de novo using recombinant DNA methodologies.

An “antibody heavy chain”, as used herein, refers to the larger of the two types of polypeptide chains present in all intact antibody molecules.

An “antibody light chain”, as used herein, refers to the smaller of the two types of polypeptide chains present in all intact antibody molecules.

The term “single chain antibody” refers to an antibody wherein the genetic information encoding the functional fragments of the antibody are located in a single contiguous length of DNA. For a thorough description of single chain antibodies, see Bird et al., 1988: Huston et al., 1988).

The term “humanized” refers to an antibody wherein the constant regions have at least about 80% or greater homology to human immunoglobulin. Additionally, some of the nonhuman, such as murine, variable region amino acid residues can be modified to contain amino acid residues of human origin. Humanized antibodies have been referred to as “reshaped” antibodies. Manipulation of the complementarity-determining regions (CDR) is a way of achieving humanized antibodies. See for example, U.S. Pat. Nos. 4,816,567; 5,482,856; 6,479,284; 6,677,436; 7,060,808; 7,906,625; 8,398,980; 8,436,150; 8,796,439; and 10,253,111; and U.S. Patent Application Publication Nos. 2003/0017534, 2018/0298087, 2018/0312588, 2018/0346564, and 2019/0151448, each of which is incorporated by reference in its entirety.

By the term “synthetic antibody” as used herein, is meant an antibody which is generated using recombinant DNA technology, such as, for example, an antibody expressed by a bacteriophage as described herein. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

As used herein, the term “secondary antibody” refers to an antibody that binds to the constant region of another antibody (the primary antibody).

The term “bioconjugate” as use herein refers to product formed from a reaction between two molecules, where one of the two molecules is a biomolecule or biomolecule derivative (e.g., an amino- or azido-functionalized nucleic acid), that forms a bond or bonds between the two molecules. The one or more bonds can be covalent, non-covalent or coordination bonds. In some embodiments, the reaction forms one or more covalent bonds between the two molecules, optionally wherein one or more atoms of either or both of the molecules form a “side product” or “leaving group” that is released during the reaction.

The term “bioconjugation” refers to a chemical reaction between two molecules, where one of the two molecules is a biomolecule or biomolecule derivative, wherein the two molecules are coupled together by one or more bonds. In some embodiments, the one or more bonds are covalent bonds and the product of the bioconjugation is a larger entity where the two molecules or monovalent derivatives thereof are bonded to one another via a covalent linkage or linker group.

The term “biomolecule” as used herein refers to peptides, proteins, nucleic acids (e.g., DNA, RNA, and derivatives thereof), saccharides (including monosaccharides, disaccharides, and polysaccharides), and lipids. The term can refer to both naturally occurring and synthesized molecules.

“Chemokine”, as used herein, refers to an intercellular signaling molecule involved in the chemotaxis of white blood cells, such as T cells.

The term “chitin” refers to (poly)GlcNAc linked in a β-1,4 fashion. Chitin is found throughout nature, for example in the exoskeletons of insects and crustacea.

The term “chitosan” refers to deacylated chitin or (poly)N-glucosamine linked in a β-1,4 fashion.

A “compound”, as used herein, refers to any type of substance or agent that is commonly considered a drug, or a candidate for use as a drug, combinations, and mixtures of the above, as well as peptides, proteins, nucleic acids, saccharides, and antibodies of the presently disclosed subject matter.

“Cytokine”, as used herein, refers to intercellular signaling molecules, the best known of which are involved in the regulation of mammalian somatic cells. A number of families of cytokines, both growth promoting and growth inhibitory in their effects, have been characterized including, for example, interleukins, interferons, and transforming growth factors. A number of other cytokines are known to those of skill in the art. The sources, characteristics, targets, and effector activities of these cytokines have been described.

As used herein, a “derivative” of a compound refers to a chemical compound that can be produced from another compound of similar structure in one or more steps (e.g., by one or more chemical or enzymatic reactions), such as by replacement of a hydrogen atom by an alkyl, acyl, or amino group. Thus, for example, derivatives can be formed via esterification of a carboxylic acid or hydroxyl group, acylation of an amino group, hydrolysis of an ester, reduction of a double bond, oxidation of a bond, and the like. The term “monovalent derivative” refers to a derivative of a molecule wherein one atom (e.g., one H atom) or chemical functional group has been removed to provide a site of covalent attachment of the molecule to another molecule or different chemical functional group, either directly or via a bivalent linker group.

As used herein, a “detectable marker” or a “reporter molecule” is an atom or a molecule that permits the specific detection of a compound comprising the marker in the presence of similar compounds without a marker. Detectable markers or reporter molecules include, e.g., radioactive isotopes, antigenic determinants, enzymes, nucleic acids available for hybridization, chromophores, fluorophores, chemiluminescent molecules, electrochemically detectable molecules, and molecules that provide for altered fluorescence-polarization or altered light-scattering.

A “fragment” or “segment” is a portion of an amino acid sequence, comprising at least one amino acid, or a portion of a nucleic acid sequence comprising at least one nucleotide. The terms “fragment” and “segment” are used interchangeably herein.

As used herein, the term “fragment”, as applied to a protein or peptide, can ordinarily be at least about 3-15 amino acids in length, at least about 15-25 amino acids, at least about 25-50 amino acids in length, at least about 50-75 amino acids in length, at least about 75-100 amino acids in length, and greater than 100 amino acids in length.

As used herein, the term “fragment” as applied to a nucleic acid, may ordinarily be at least about 20 nucleotides in length, typically, at least about 50 nucleotides, more typically, from about 50 to about 100 nucleotides, in some embodiments, at least about 100 to about 200 nucleotides, in some embodiments, at least about 200 nucleotides to about 300 nucleotides, yet in some embodiments, at least about 300 to about 350, in some embodiments, at least about 350 nucleotides to about 500 nucleotides, yet in some embodiments, at least about 500 to about 600, in some embodiments, at least about 600 nucleotides to about 620 nucleotides, yet in some embodiments, at least about 620 to about 650, and most in some embodiments, the nucleic acid fragment will be greater than about 650 nucleotides in length.

As used herein, a “functional” molecule is a molecule in a form in which it exhibits a property or activity by which it is characterized.

The term “glycopeptide” refers to a molecule comprising a peptide, e.g., a cyclic or multicyclic peptide, and further comprising a carbohydrate moiety or moieties covalently attached to a side chain of one or more amino acid residues of the peptide. As used herein, the term “glycopeptide” refers to those glycopeptides wherein at least one of the carbohydrate moieties comprises a primary aminoalkyl group or a secondary amino alkyl group. For example, in some embodiments, the glycopeptide can be a glycopeptide antibiotic comprising one or more primary or secondary aminoalkyl groups, such as, but not limited to vancomycin. In addition to vancomycin, other exemplary glycopeptides include, but are not limited to, actaplanin, actinoidin, avoparcin, balhimycin, chloropolysporin c, eremonmycin, galacardin a, helvecardin a, and helvecardin b.

The term “glycosaminoglycan” refers to long heteropolysaccharide molecules containing repeating disaccharide units. The disaccharide units can comprise modified aminosugars: D-, N-acetylgalactosamine or D-GlcNAc and an uronic acid such as D-glucuronate or L-iduronate. Among other functions, glucosaminoglycans (GAGs) serve as a lubricating fluid in the joints. Exemplary GAGs include, but are not limited to, hyaluronic acid, dermatan sulfate, chondroitin sulfate, heprin, heparan sulfate, and keratan sulfate.

“Homologous” as used herein, refers to the subunit sequence similarity between two polymeric molecules, e.g., between two nucleic acid molecules, e.g., two DNA molecules or two RNA molecules, or between two polypeptide molecules. When a subunit position in both of the two molecules is occupied by the same monomeric subunit, e.g., if a position in each of two DNA molecules is occupied by adenine, then they are homologous at that position. The homology between two sequences is a direct function of the number of matching or homologous positions, e.g., if half (e.g., five positions in a polymer ten subunits in length) of the positions in two compound sequences are homologous then the two sequences are 50% homologous, if 90% of the positions, e.g., 9 of 10, are matched or homologous, the two sequences share 90% homology. By way of example, the DNA sequences 5′-ATTGCC-3′ and 5′-TATGGC-3′ share 50% homology.

As used herein, “homology” is used synonymously with “identity”. The determination of percent identity between two nucleotide or amino acid sequences can be accomplished using a mathematical algorithm. For example, a mathematical algorithm useful for comparing two sequences is the algorithm of Karlin & Altschul (1990) Methods for assessing the statistical significance of molecular sequence features by using general scoring schemes. Proc Natl Acad Sci USA 87:2264-2268, modified as in Karlin & Altschul (1993) Applications and statistics for multiple high-scoring segments in molecular sequences. Proc Natl Acad Sci USA 90:5873-5877). This algorithm is incorporated into the NBLAST and XBLAST programs (see Altschul et al. (1990a) Basic local alignment search tool. J Mol Biol 215:403-410; Altschul et al. (1990b) Protein database searches for multiple alignments. Proc Natl Acad Sci USA 87:14:5509-5513, and can be accessed, for example at the National Center for Biotechnology Information (NCBI) world wide web site. BLAST nucleotide searches can be performed with the NBLAST program (designated “blastn” at the NCBI web site), using the following parameters: gap penalty=5; gap extension penalty=2; mismatch penalty=3; match reward=1; expectation value 10.0; and word size=11 to obtain nucleotide sequences homologous to a nucleic acid described herein. BLAST protein searches can be performed with the XBLAST program (designated “blastn” at the NCBI web site) or the NCBI “blastp” program, using the following parameters: expectation value 10.0. BLOSUM62 scoring matrix to obtain amino acid sequences homologous to a protein molecule described herein. To obtain gapped alignments for comparison purposes, Gapped BLAST can be utilized as described in Altschul et al. (1997) Gapped BLAST and PSI-BLAST: a new generation of protein database search programs. Nucleic Acids Res 25:3389-3402. Alternatively, PSI-Blast or PHI-Blast can be used to perform an iterated search which detects distant relationships between molecules (Id.) and relationships between molecules which share a common pattern. When utilizing BLAST, Gapped BLAST, PSI-Blast, and PHI-Blast programs, the default parameters of the respective programs (e.g., XBLAST and NBLAST) can be used.

The percent identity between two sequences can be determined using techniques similar to those described above, with or without allowing gaps. In calculating percent identity, typically exact matches are counted.

As used herein, the term “hybridization” is used in reference to the pairing of complementary nucleic acids. Hybridization and the strength of hybridization (i.e., the strength of the association between the nucleic acids) is impacted by such factors as the degree of complementarity between the nucleic acids, stringency of the conditions involved, the length of the formed hybrid, and the G:C ratio within the nucleic acids.

The term “hyaluronic acid” refers to a mucopolysaccharide comprising alternating subunits of D-glucuronic acid and D-N-acetyl glucosamine linked by β-(1-4)-glycosidic linkages. Hyaluronic acid is commercially available in several molecular weight ranges spanning from about 50,000 Daltons to about 8×10⁶ Daltons. Hyaluronic acid is also available as a sodium salt and is a dried, highly purified substance.

The term “ionic liquid” as used herein refers a molecule (a salt) which is in the form of a liquid at temperatures below about 100° C., where at least part of the liquid is in the form of ions. Ionic liquids are polar and aprotic. In some embodiments, the ionic liquid comprises a cation selected from the group consisting of imidazolium, alkyl-imidazole, alkyl-ammonium, alkyl-sulfonium, alkyl-piperidinium, alkyl-pyridinium, alkyl-phosphonium, and alkyl-pyrrolidinium; and an anion selected from the group consisting of carboxylate, halide, fulminate, persulfate, sulfate, sulfites, phosphates, phosphites, nitrate, nitrites, hypochlorite, chlorite, bicarbonates, imides, sulfonimides, and borates.

An “isolated nucleic acid” refers to a nucleic acid segment or fragment, which has been separated from sequences, which flank it in a naturally occurring state, e.g., a DNA fragment that has been removed from the sequences, which are normally adjacent to the fragment, e.g., the sequences adjacent to the fragment in a genome in which it naturally occurs. The term also applies to nucleic acids, which have been substantially purified, from other components, which naturally accompany the nucleic acid, e.g., RNA or DNA, or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (e.g., as a cDNA or a genomic or cDNA fragment produced by PCR or restriction enzyme digestion) independent of other sequences. It also includes a recombinant DNA, which is part of a hybrid gene encoding additional polypeptide sequence.

As used herein, a “ligand” is a compound that specifically binds to a target compound. A ligand (e.g., an antibody) “specifically binds to” or “is specifically immunoreactive with” a compound when the ligand functions in a binding reaction which is determinative of the presence of the compound in a sample of heterogeneous compounds. Thus, under designated assay (e.g., immunoassay) conditions, the ligand binds preferentially to a particular compound and does not bind to a significant extent to other compounds present in the sample. For example, an antibody specifically binds under immunoassay conditions to an antigen bearing an epitope against which the antibody was raised. A variety of immunoassay formats can be used to select antibodies specifically immunoreactive with a particular antigen. For example, solid-phase ELISA immunoassays are routinely used to select monoclonal antibodies specifically immunoreactive with an antigen. See Harlow & Lane, 1988 for a description of immunoassay formats and conditions that can be used to determine specific immunoreactivity.

As used herein, the term “linkage” refers to a connection between two groups, such as that formed between a biomolecule substrate and another molecule during a bioconjugation reaction. The connection can be either covalent or non-covalent, including but not limited to ionic bonds, hydrogen bonding, and hydrophobic/hydrophilic interactions. In some embodiments, the connection is via covalent bonds, e.g., via a bivalent chemical functional group such as an urea linkage.

As used herein, the term “mass spectrometry” (MS) refers to a technique for the identification and/or quantitation of molecules in a sample. MS includes ionizing the molecules in a sample, forming charged molecules; separating the charged molecules according to their mass-to-charge ratio; and detecting the charged molecules. MS allows for both the qualitative and quantitative detection of molecules in a sample. The molecules can be ionized and detected by any suitable means known to one of skill in the art. Some examples of mass spectrometry are “tandem mass spectrometry” or “MS/MS,” which are the techniques wherein multiple rounds of mass spectrometry occur, either simultaneously using more than one mass analyzer or sequentially using a single mass analyzer. The term “mass spectrometry” can refer to the application of mass spectrometry to protein analysis. In some embodiments, electrospray ionization (ESI) and matrix-assisted laser desorption/ionization (MALDI) can be used in this context.

The term “nucleic acid” refers to molecules composed of monomeric nucleotides. Thus, “nucleic acid” includes ribonucleic acids (RNA), deoxyribonucleic acids (DNA), single-stranded nucleic acids (ssDNA), double-stranded nucleic acids (dsDNA), small interfering ribonucleic acids (siRNA) and microRNAs (miRNA). Nucleic acids also include antisense nucleic acid, ribozymes, aptamers, and spiegelmers. A nucleic acid can also comprise any combination of these elements in a single molecule.

Thus, as used herein, the term “nucleic acid” encompasses RNA as well as single and double stranded DNA and cDNA. Furthermore, the terms, “nucleic acid”, “DNA”, “RNA” and similar terms also include nucleic acid analogs, i.e. analogs having other than a phosphodiester backbone. For example, the so called “peptide nucleic acids”, which are known in the art and have peptide bonds instead of phosphodiester bonds in the backbone, are considered within the scope of the presently disclosed subject matter. Thus, by “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, bridged phosphoramidate, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine, and uracil).

Conventional notation is used herein to describe polynucleotide sequences: the left-hand end of a single-stranded polynucleotide sequence is the 5′-end: the left-hand direction of a double-stranded polynucleotide sequence is referred to as the 5′-direction. The direction of 5′ to 3′ addition of nucleotides to nascent RNA transcripts is referred to as the transcription direction. The DNA strand having the same sequence as an mRNA is referred to as the “coding strand”; sequences on the DNA strand which are located 5′ to a reference point on the DNA are referred to as “upstream sequences”; sequences on the DNA strand which are 3′ to a reference point on the DNA are referred to as “downstream sequences”.

The term “nucleic acid construct”, as used herein, encompasses DNA and RNA sequences encoding the particular gene or gene fragment desired, whether obtained by genomic or synthetic methods.

Unless otherwise specified, a “nucleotide sequence encoding an amino acid sequence” includes all nucleotide sequences that are degenerate versions of each other and that encode the same amino acid sequence. Nucleotide sequences that encode proteins and RNA can include introns.

The term “oligonucleotide” typically refers to short polynucleotides, generally, no greater than about 50 nucleotides. It will be understood that when a nucleotide sequence is represented by a DNA sequence (i.e., A, T, G, C), this also includes an RNA sequence (i.e., A, U, G, C) in which “U” replaces “T”.

By describing two polynucleotides as “operably linked” is meant that a single-stranded or double-stranded nucleic acid moiety comprises the two polynucleotides arranged within the nucleic acid moiety in such a manner that at least one of the two polynucleotides is able to exert a physiological effect by which it is characterized upon the other. By way of example, a promoter operably linked to the coding region of a gene is able to promote transcription of the coding region.

The terms “polymer” and “polymeric” refer to chemical structures that have repeating units (i.e., multiple copies of a given chemical substructure). Polymers can be formed from polymerizable monomers. A polymerizable monomer is a molecule that comprises one or more moieties that can react to form bonds (e.g., covalent or coordination bonds) with moieties on other molecules of polymerizable monomer. In some embodiments, each polymerizable monomer molecule can bond to two or more other molecules/moieties. In some cases, a polymerizable monomer will bond to only one other molecule, forming a terminus of the polymeric material.

As used herein “organic polymers” are those that do not include silica or metal atoms in their repeating units. Exemplary organic polymers include polyvinylpyrrolidone (PVO), polyesters, polyamides, polyethers, polydienes, and the like. Some organic polymers contain biodegradable linkages, such as esters or amides, such that they can degrade overtime under biological conditions. The term “hydrophilic polymer” as used herein generally refers to hydrophilic organic polymers, such as but not limited to, poly vinylpyrrolidone (PVP), polyvinylmethylether, polymethyloxazoline, polyethyloxazoline, polyhydroxy-propyloxazoline, polyhydroxypropylmethacrylamide, polymethyacrylamide, polydimethylacrylamide, polyhydroxypropylmethacrylate, polyhydroxy-ethylacrylate, hydroxymethylcellulose, hydroxyethylcellulose, polyethylene-imine (PEI), polyethyleneglycol (i.e., PEG) or another hydrophilic poly(alkyleneoxide), polyglycerine, and polyaspartamide. The term “hydrophilic” refers to the ability of a molecule or chemical species to interact with water. Thus, hydrophilic polymers are typically polar or have groups that can hydrogen bond to water.

A “polynucleotide” means a single strand or parallel and anti-parallel strands of a nucleic acid. Thus, a polynucleotide can be either a single-stranded or a double-stranded nucleic acid.

“Polypeptide” refers to a polymer composed of amino acid residues, related naturally occurring structural variants, and synthetic non-naturally occurring analogs thereof linked via peptide bonds, related naturally occurring structural variants, and synthetic non-naturally occurring analogs thereof.

“Synthetic peptides or polypeptides” means a non-naturally occurring peptide or polypeptide. Synthetic peptides or polypeptides can be synthesized, for example, using an automated polypeptide synthesizer. Various solid phase peptide synthesis methods are known to those of skill in the art.

The term “protein” typically refers to large polypeptides (e.g., greater than 50 amino acid residues), while “peptide” can be used to refer to smaller polypeptides (e.g., 2 to 50 amino acid residues). Conventional notation is used herein to portray polypeptide sequences: the left-hand end of a polypeptide sequence is the amino-terminus: the right-hand end of a polypeptide sequence is the carboxyl-terminus.

As used herein, “protecting group” with respect to a terminal amino group refers to a terminal amino group of a peptide, which terminal amino group is coupled with any of various amino-terminal protecting groups traditionally employed in peptide synthesis. Such protecting groups include, for example, acyl protecting groups such as formyl, acetyl, benzoyl, trifluoroacetyl, succinyl, and methoxysuccinyl; aromatic urethane protecting groups such as benzyloxycarbonyl; and aliphatic urethane protecting groups, for example, tert-butoxycarbonyl or adamantyloxycarbonyl. See Gross & Mienhofer, 1981 for suitable protecting groups.

As used herein, “protecting group” with respect to a terminal carboxy group refers to a terminal carboxyl group of a peptide, which terminal carboxyl group is coupled with any of various carboxyl-terminal protecting groups. Such protecting groups include, for example, tert-butyl, benzyl, or other acceptable groups linked to the terminal carboxyl group through an ester or ether bond.

The terms “saccharide”, “carbohydrate,” “sugar.” and “starch” as used herein refer to a molecule comprising one or more units derived from a monosaccharide. The terms saccharide as used herein can refer to all carbohydrate, saccharide, sugar, or starch molecules of any size, structure, or function. A saccharide can be a monosaccharide or a single sugar molecule. Two or more monosaccharides can be joined by one or more glycosidic bonds to produce higher order saccharides. A disaccharide is comprised of two monosaccharides, an oligosaccharide is comprised of about 3 to about 10 monosaccharides, and a polysaccharide is comprised of about 10 or more monosaccharides.

Representative saccharides include, by way of illustration, hexoses such as D-glucose, D-mannose, D-xylose, D-galactose, vancosamine, 3-desmethyl-vancosamine, 3-epi-vancosamine, 4-epi-vancosamine, acosamine, actinosamine, daunosamine, 3-epi-daunosamine, ristosamine, N-methyl-D-glucamine, D-glucuronic acid, N-acetyl-D-glucosamine, N-acetyl-D-galactosamine, sialyic acid, iduronic acid, L-fucose, and the like; pentoses such as D-ribose or D-arabinose: ketoses such as D-ribulose or D-fructose; disaccharides such as 2-O-(α-L-vancosaminyl)-β-D-glucopyranose, 2-O-(3-desmethyl-α-L-vancosaminyl)-β-D-glucopyranose, sucrose, lactose, or maltose; derivatives such as acetals, amines, acylated, sulfated and phosphorylated sugars; and oligosaccharides having from 3 to 10 saccharide units.

As used herein the term “small molecule” refers to a molecule with a molecular weight of less than about 2000 Da, optionally less than about 1500 Da, less than about 1000 Da, less than about 900 Da, less than about 800 Da, less than about 750 Da, less than about 700 Da, less than about 650 Da, or less than about 600 Da. In some embodiments, the term “small molecule” is used to refer to a non-polymeric or non-oligomeric compound. In some embodiments, the small molecule is a synthetic molecule. In some embodiments, the small molecule is a naturally occurring molecule, e.g., an alkaloid, an antibiotic, etc.

As used herein the term “alkyl” refers to C₁₋₂₀ inclusive, linear (i.e., “straight-chain”), branched, or cyclic, saturated or at least partially and in some cases fully unsaturated (i.e., alkenyl and alkynyl) hydrocarbon chains, including for example, methyl, ethyl, propyl, isopropyl, butyl, isobutyl, tert-butyl, pentyl, hexyl, octyl, ethenyl, propenyl, butenyl, pentenyl, hexenyl, octenyl, butadienyl, propynyl, butynyl, pentynyl, hexynyl, heptynyl, and allenyl groups. “Branched” refers to an alkyl group in which a lower alkyl group, such as methyl, ethyl or propyl, is attached to a linear alkyl chain. In some embodiments, the alkyl group is “lower alkyl.” “Lower alkyl” refers to an alkyl group having 1 to about 8 carbon atoms (i.e., a C₁₋₈ alkyl), e.g., 1, 2, 3, 4, 5, 6, 7, or 8 carbon atoms. In some embodiments, the alkyl is “higher alkyl.” “Higher alkyl” refers to an alkyl group having about 10 to about 20 carbon atoms, e.g., 10, 11, 12, 13, 14, 15, 16, 17, 18, 19, or 20 carbon atoms. In certain embodiments, “alkyl” refers, in particular, to C₁₋₈ straight-chain alkyls. In other embodiments, “alkyl” refers, in particular, to C₁₋₈ branched-chain alkyls.

Alkyl groups can optionally be substituted (a “substituted alkyl”) with one or more alkyl group substituents, which can be the same or different. The term “alkyl group substituent” includes but is not limited to alkyl, substituted alkyl, halo, arylamino, acyl, hydroxyl, aryloxyl, alkoxyl, alkylthio, arylthio, aralkyloxyl, aralkylthio, carboxyl, alkoxycarbonyl, oxo, and cycloalkyl. There can be optionally inserted along the alkyl chain one or more oxygen, sulfur or substituted or unsubstituted nitrogen atoms, wherein the nitrogen substituent is hydrogen, lower alkyl (also referred to herein as “alkylaminoalkyl”), or aryl.

Thus, as used herein, the term “substituted alkyl” includes alkyl groups, as defined herein, in which one or more atoms or functional groups of the alkyl group are replaced with another atom or functional group, including for example, alkyl, substituted alkyl, halogen, aryl, substituted aryl, alkoxyl, hydroxyl, nitro, amino, alkylamino, dialkylamino, sulfate, and mercapto.

The term “aryl” is used herein to refer to an aromatic moiety that can be a single aromatic ring, or multiple aromatic rings that are fused together, linked covalently, or linked to a common group, such as, but not limited to, a methylene or ethylene moiety. The common linking group also can be a carbonyl, as in benzophenone, or oxygen, as in diphenylether, or nitrogen, as in diphenylamine. The term “aryl” specifically encompasses heterocyclic aromatic compounds. The aromatic ring(s) can comprise phenyl, naphthyl, biphenyl, diphenylether, diphenylamine and benzophenone, among others. In particular embodiments, the term “aryl” means a cyclic aromatic comprising about 5 to about 10 carbon atoms, e.g., 5, 6, 7, 8, 9, or 10 carbon atoms, and including 5- and 6-membered hydrocarbon and heterocyclic aromatic rings.

The aryl group can be optionally substituted (a “substituted aryl”) with one or more aryl group substituents, which can be the same or different, wherein “aryl group substituent” includes alkyl, substituted alkyl, aryl, substituted aryl, aralkyl, hydroxyl, alkoxyl, aryloxyl, aralkyloxyl, carboxyl, carbonyl, acyl, halo, nitro, alkoxycarbonyl, aryloxycarbonyl, aralkoxycarbonyl, acyloxyl, acylamino, aroylamino, carbamoyl, alkylcarbamoyl, dialkylcarbamoyl, arylthio, alkylthio, alkylene, and —NR′R″, wherein R′ and R″ can each be independently hydrogen, alkyl, substituted alkyl, aryl, substituted aryl, and aralkyl.

Thus, as used herein, the term “substituted aryl” includes aryl groups, as defined herein, in which one or more atoms or functional groups of the aryl group are replaced with another atom or functional group, including for example, alkyl, substituted alkyl, halogen, aryl, substituted aryl, alkoxyl, hydroxyl, nitro, amino, alkylamino, dialkylamino, sulfate, and mercapto.

Specific examples of aryl groups include, but are not limited to, cyclopentadienyl, phenyl, furan, thiophene, pyrrole, pyran, pyridine, imidazole, benzimidazole, isothiazole, isoxazole, pyrazole, pyrazine, triazine, pyrimidine, quinoline, isoquinoline, indole, carbazole, and the like.

The term “heteroaryl” refers to aryl groups wherein at least one atom of the backbone of the aromatic ring or rings is an atom other than carbon. Thus, heteroaryl groups have one or more non-carbon atoms selected from the group including, but not limited to, nitrogen, oxygen, and sulfur.

As used herein, the term “acyl” refers to an organic carboxylic acid group wherein the —OH of the carboxyl group has been replaced with another substituent (i.e., as represented by RCO—, wherein R is an alkyl or an aryl group as defined herein). As such, the term “acyl” specifically includes arylacyl groups, such as an acetylfuran and a phenacyl group. Specific examples of acyl groups include acetyl and benzoyl.

“Cyclic” and “cycloalkyl” refer to a non-aromatic mono- or multicyclic ring system of about 3 to about 10 carbon atoms, e.g., 3, 4, 5, 6, 7, 8, 9, or 10 carbon atoms. The cycloalkyl group can be optionally partially unsaturated. The cycloalkyl group also can be optionally substituted with an alkyl group substituent as defined herein, oxo, and/or alkylene. There can be optionally inserted along the cyclic alkyl chain one or more oxygen, sulfur or substituted or unsubstituted nitrogen atoms, wherein the nitrogen substituent is hydrogen, alkyl, substituted alkyl, aryl, or substituted aryl, thus providing a heterocyclic group. Representative monocyclic cycloalkyl rings include cyclopentyl, cyclohexyl, and cycloheptyl. Multicyclic cycloalkyl rings include adamantyl, octahydronaphthyl, decalin, camphor, camphane, and noradamantyl.

The terms “heterocycle” or “heterocyclic” refer to cycloalkyl groups (i.e., non-aromatic, cyclic groups as described hereinabove) wherein one or more of the backbone carbon atoms of a cyclic ring is replaced by a heteroatom (e.g., nitrogen, sulfur, or oxygen). Examples of heterocycles include, but are not limited to, tetrahydrofuran, tetrahydropyran, morpholine, dioxane, piperidine, piperazine, and pyrrolidine.

“Alkylene” refers to a straight or branched bivalent aliphatic hydrocarbon group having from 1 to about 20 carbon atoms, e.g., 1, 2, 3, 4, 5, 6, 7, 8, 9, 10, 11, 12, 13, 14, 15, 16, 17, 18, 19, or 20 carbon atoms. The alkylene group can be straight, branched or cyclic. The alkylene group also can be optionally unsaturated and/or substituted with one or more “alkyl group substituents.” There can be optionally inserted along the alkylene group one or more oxygen, sulfur or substituted or unsubstituted nitrogen atoms (also referred to herein as “alkylaminoalkyl”), wherein the nitrogen substituent is alkyl as previously described. Exemplary alkylene groups include methylene (—CH₂—); ethylene (—CH₂—CH₂—); propylene (—(CH₂)₃—); cyclohexylene (—C₆H₁₀—); —CH═CH—CH═CH—; —CH═CH—CH₂—; —(CH₂)_{q}—N(R)—(CH₂)ᵣ—, wherein each of q and r is independently an integer from 0 to about 20, e.g., 0, 1, 2, 3, 4, 5, 6, 7, 8, 9, 10, 11, 12, 13, 14, 15, 16, 17, 18, 19, or 20, and R is hydrogen or lower alkyl; methylenedioxyl (—O—CH₂—O—); and ethylenedioxyl (—O—(CH₂)₂—O—). An alkylene group can have about 2 to about 3 carbon atoms and can further have 6-20 carbons.

The term “arylene” refers to a bivalent aryl group.

“Alkoxyl” or “alkoxy” refers to an alkyl-O— group wherein alkyl is as previously described. The term “alkoxyl” as used herein can refer to, for example, methoxyl, ethoxyl, propoxyl, isopropoxyl, butoxyl, t-butoxyl, and pentoxyl. The term “oxyalkyl” can be used interchangeably with “alkoxyl”.

“Aralkyl” refers to an aryl-alkyl-group wherein aryl and alkyl are as previously described, and included substituted aryl and substituted alkyl. Exemplary aralkyl groups include benzyl, phenylethyl, and naphthylmethyl. The term “aralkylene” refers to a bivalent aralkyl group.

The term “amino” refers to the —NR′R″ group, wherein R′ and R″ are each independently selected from the group including H and substituted and unsubstituted alkyl, cycloalkyl, heterocycle, aralkyl, aryl, and heteroaryl. In some embodiments, the amino group is —NH₂. The term “amine” can be used to refer to a compound comprising an amino group or to the amino group itself.

The terms “aminoalkyl”, “alkylamine”, and “alkylamino” as used herein refer to an group having the formula NH(R′)-alkyl, wherein the R′ is selected from H and substituted and unsubstituted alkyl or cycloalkyl; and wherein alkyl is substituted or unsubstituted alkyl with one or more alkyl group substituents. In some embodiments, the term “aminoalkyl” refers to a primary aminoalkyl group, i.e., having the formula NH₂-alkyl. In some embodiments, the term “aminoalkyl” refers to a secondary aminoalkyl group, i.e., having the formula NH(R′)-alkyl, where R′ is substituted or unsubstituted alkyl or cycloalkyl. In some embodiments, “aminoalkyl” refers to both primary and secondary aminoalkyl groups.

The term “carbonyl” refers to the —(C═O)— or a double bonded oxygen substituent attached to a carbon atom of a previously named parent group.

The terms “carboxyl” and “carboxylic acid” refers to the —C(═O)OH group. Unless specified otherwise, the terms also encompass “carboxylate”, i.e., —C(═O)O—.

The terms “halo”, “halide”, or “halogen” as used herein refer to fluoro, chloro, bromo, and iodo groups.

The terms “hydroxyl” and “hydroxy” refer to the —OH group.

The term “azide” refers to a group or compound comprising an —N₃ moiety.

The term “urea” as used herein refers to a group or a molecule comprising the formula —NH—C(═O)—NH—.

The term “amide” refers to a group or molecule comprising the formula —NH—C═O)—R or —NH—(C═O)—R′—, wherein R is substituted or unsubstituted alkyl, cycloalkyl, aralkyl, or aryl and R′ is a substituted or unsubstituted alkylene, cycloalkylene, aralkylene, or arylene.

The term “phosphine” as used herein refers to a molecule comprising the formula P(R)₃, wherein each R is independently selected from H and substituted or unsubstituted alkyl, cycloalkyl, aralkyl, and aryl. The term “triarylphosphine” refers to a molecule comprising the formula P(R)₃, wherein each R is substituted or unsubstituted aryl.

The terminology used herein is for the purpose of describing the particular versions or embodiments only and is not intended to limit the scope of the presently disclosed subject matter. All publications mentioned herein are incorporated by reference in their entirety.

### II. General Considerations

Biomolecule modification can play a variety of roles in diverse medical applications, and selective chemical reactions can be useful tools for the formation of a covalent bond between a biomolecule (e.g., a proteins) and new functionalities. For instance, antibodies are proteins produced in response to a particular antigen and which can selectively bind to the particular antigen. Antibodies and drugs can be combined to create antibody-drug conjugates (ADCs). ADCs can be used as a form of nanomedicine, in which the antibody acts as a nanoscale carrier to selectively deliver and release, for example, a cytotoxic or other therapeutic molecule to a target antigen or target-antigen associated entity (e.g., a cancer cell expressing a target antigen). Examples of ADCs include gemtuzumab ozogamicin, used in the treatment of acute myeloid leukemia, and brentuximab vedotin, used in the treatment of Hodgkin lymphoma. In addition to therapeutic compounds, protein modification is also useful for the introduction of a visualization handle providing for the creation of diagnostic tools, e.g., for diagnosing cancer. In some embodiments, the visualization handle can comprise a radioisotope and the resulting modified protein can be used as a diagnostic in methods using positron emission tomography (PET). Biomolecule conjugation has also been used to add entities that can prolong circulating half-life and/or reduce antigenicity. Accordingly, covalent attachment of polyethylene glycol chains (referred to as PEGylation) and/or other polymers to proteins is another exemplary type of protein modification. Examples of PEGylated protein therapeutics include those sold under the tradenames PLEGRIDY® (Biogen MA Inc., Cambridge, Mass., United States of America), used in the treatment of multiple sclerosis, and SYLATRON™ (Merck. Sharp & Dohme Corp., Whitehouse Station, N.J. United States of America), used in the treatment of melanoma.

While a diverse set of design strategies have produced various chemical tools for biomolecule labeling in aqueous media, the development of nonaqueous, biomolecule-compatible media for bioconjugation has significantly lagged behind. One impediment to the creation of robust conjugation of biomolecules such as proteins and peptides with other molecules, e.g., cytotoxins, is the difficulty in performing selective chemical reactions in an aqueous solution. On the other hand, the use of organic solvents for bioconjugation reactions can be difficult due to their general incompatibility with proteins and other biomolecules.

According to one aspect of the presently disclosed subject matter, a bioconjugation strategy using biomolecule-compatible non-aqueous media, also referred to herein as “Bioconjugation in Nonaqueous-Driven Reaction Solvent” or “BINDRS” is provided. In some embodiments, the presently disclosed subject matter provides a method of performing a bioconjugation reaction in a reaction mixture using an ionic liquid solvent or solvents. In some embodiments, the reaction mixture comprises a solvent or solvent mixture comprising no more than about 6% of an aqueous solvent. For example, it is demonstrated hereinbelow that aprotic ionic liquid serves as a reaction solvent for protein bioconjugation without noticeable loss of the biomolecule functions. The presently disclosed strategy with an untraditional ionic liquid medium can provide untapped opportunities for expanding the scope of chemical approaches for bioconjugation.

Ionic liquids emerged as an alternative medium for studying biomolecules by virtue of their organic framework combined with an ionic nature akin to that of biological buffers. Salts that are in a liquid state at <100° C., ionic liquids have been examined as substitutes for organic solvents in various chemistry areas.²¹⁻²³ At the same time, there is evidence indicating compatibility of various biomolecules with ionic liquids,²⁴⁻²⁶ including the example of the better thermal stability in ionic liquid than aqueous solution.²⁷ However, the presently disclosed subject matter is believed to be the first utilization of an ionic liquid for protein bioconjugation.

The presently disclosed ionic liquid bioconjugation approach led, for example, to discovery of a phosphine-mediated amine-azide coupling reaction in ionic liquids which forges a stable urea linkage on unprotected peptides and proteins. For comparison, the same reaction was performed in an aqueous solution and in organic solvents. As disclosed herein, it was observed that the peptide was only modified with the azide-containing compound when using the ionic liquid solvent. The bioconjugation reaction was further screened using various exemplary azides compounds and various peptide and protein substrates, such as daptomycin (an antibiotic used to treat serious bacterial infections) and angiotensin II (a hormone that helps maintain blood pressure and fluid balance in the body). The bioconjugation reactions were also performed using antibodies as the biomolecule substrate to form an exemplary ADC, which did not lose functionality after exposure to the ionic liquid medium.

In addition to bioconjugations of peptides and proteins, the presently disclosed strategy was also employed with other biomolecules, such as nucleic acids and saccharides (e.g., aminosaccharides), providing access to additional functionalized biomolecules. For instance, chemically functionalized deoxyribonucleic acids (DNA) have become invaluable in various applications such as molecular beacons,⁶⁴⁻⁶⁵ polymerase chain reaction (PCR) technologies,⁶⁶ deoxyribozymes (DNAzymes),^{67,68} asymmetric catalysis,⁶⁹ nanotechnolgy,⁷⁰ and therapeutics⁷¹ such as antisense sequences,^{72,73} transcription-factor decoys,^{74,75} and CpG motifs.⁷⁶⁻⁷⁷. While chemical synthesis methods for nucleic acids can facilitate the introduction of desired functionalities⁷⁸ and post-synthetic modification methods,⁷⁹⁻⁸⁵ including bio-inspired enzymatic post-synthetic labeling technologies,⁸⁶ have proven useful, site-specific chemical modification of nucleic acids remains a challenge.

Owing to the ionic nature and resulting solubility of nucleic acids (e.g., DNAs), water or aqueous buffer is often a common choice for bioconjugation processes. However, aqueous reaction conditions can have negative effects, which include the low solubility and/or stability of labeling reagents and sluggish kinetics. Ionic liquids have been successfully employed for dissolution and stabilization of various DNA molecules.^{25,105} However, ionic liquids have not been used in DNA bioconjugation processes. As described herein bioconjugation processes in ionic liquid solvents represents an alternative strategy to address the compatibility issue between DNAs and organic chemical reactions.

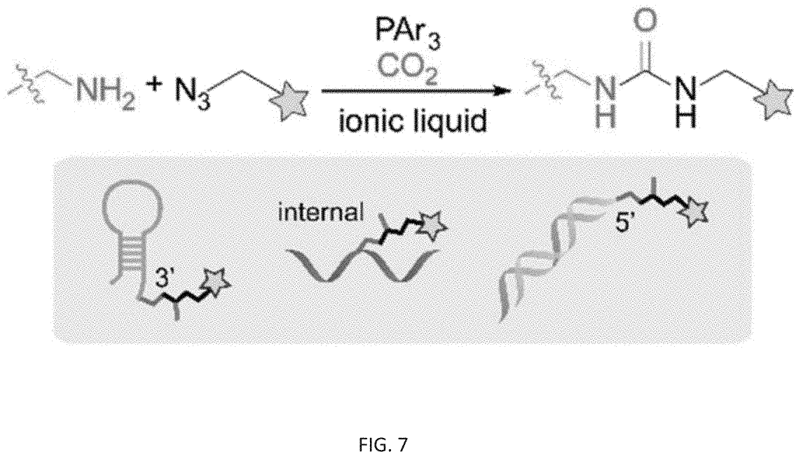

More particularly, as described herein, it was found that phosphine-mediated amine-azide coupling in ionic liquids had no observable effect on native DNA functional groups such as DNA bases, phosphate backbone and ribose groups. This capability provided the possibility of site-specific modification at a desired location in DNA through incorporation of an exogenous alkylamine group. See FIG. 7. Reagent screening revealed the wide tolerance of the amine-azide coupling of various chemical functionalities, enabling preparation of a variety of functionalized DNA aptamers such as fluorophore, cholesterol, and biotin conjugates. The aptamer conjugates were successfully used in staining experiments of cancer cell lines, validating the compatibility of the ionic liquid-based modification processes with the biomolecule.

Chemical functionalization of carbohydrates also provides an important chemical tool at the interface of chemistry and biology research areas. A saccharide motif is often found in therapeutically important small molecules (e.g. anti-tumor agents, vaccines, and antibiotics),^{106,107} and attachment of new functionality through bioconjugation is an approach to increase their efficacy for treatment.^{108,109} The utility of carbohydrate bioconjugation is not limited only for small mono- or oligosaccharides, however. Conjugates of structurally and functionally diverse polysaccharides, such as chitosan,¹¹⁰ alginate,¹¹¹ cellulose,¹¹² dextran,¹¹³ and hyaluronate¹¹⁴ have also demonstrated their potential for various applications. In addition, carbohydrate-targeting bioconjugation is an emerging approach for preparation of glycoprotein conjugates including antibody-drug conjugates, where oligosaccharide units could act as reactive handles for selective chemical modification processes.^{115,116} Further, the advent of bioorthogonal chemistry has led to increased interest in approaches for labeling saccharides of interest in a complicated mixture of biomolecules, including in cell and in vivo environments, to interrogate their roles in living systems.¹¹⁷⁻¹²²

Challenges of selective labeling of saccharides stem from their chemical diversity with highly oxygen-rich nature. The polyol structure not only hinders selective labeling of a target functional group, but the presence of multiple hydrophilic groups also results in their poor solubility in organic solvents.¹²³ In addition to difficulties at the molecular level, each polysaccharide tends to form their unique, intricate three-dimensional structures and aggregates, which can prevent generalization of a single method to different polysaccharides. Selective labeling of saccharides in glycoproteins is also a challenge, as ideally, these methods would include suppression of the reactivity of the labeling reagents toward a number of nucleophilic functional groups (e.g., thiol, imidazole, and phenol) present in the polypeptide portions of these molecules.¹²⁴

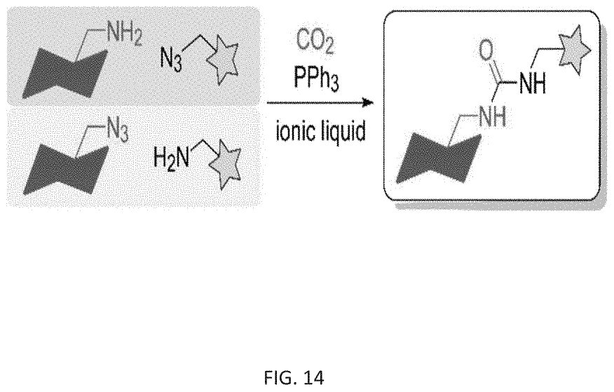

A phosphine-mediated chemical labeling strategy in ionic liquid for bioconjugation of amine- and azide-containing saccharides is described herein. See FIG. 14. Isotope labeling, crystallographic, and chromatographic/spectroscopic data confirmed that a urea group is formed through the amine-azide coupling by incorporating carbon dioxide from the atmosphere. In addition, in order to separate the target saccharide from ionic liquid and excess labeling reagents that are often incompatible with the down-stream analysis, multiple post-reaction cleanup methods were established to handle a variety of types of carbohydrate derivatives. A survey of chemoselectivity of the phosphine-mediated chemistry toward different types of amines revealed its selectivity toward amines comprising primary and secondary aminoalkyl groups, and this capability was applied for labeling of saccharides such as anti-tumor agent, antibiotics, and polysaccharide substrates containing the active amine groups. In addition to the modification of amino-carbohydrates, azide-containing saccharides were selectively modified by amine reagents, as described further hereinbelow. As also described hereinbelow, tagging of azide-labeled saccharides through amine-azide coupling is possible, even in cell lysate. It is believed that the presently disclosed subject matter represents the first bioconjugations of saccharides in ionic liquid that do not involve traditional chemical reactions (e.g. amide condensation and simple substitution reactions).

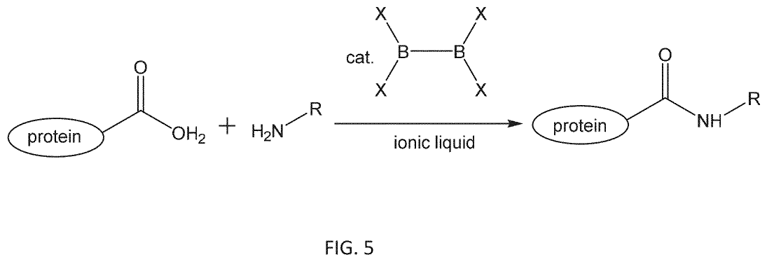

As well as performing amine-azide bioconjugation reactions, bioconjugation reactions were also performed between the carboxylic acid groups of biomolecule substrates (e.g., carboxylic acid groups of proteins) and the amino groups of functionalized molecules comprising amines using a diboron catalyst to form amide linkages. See FIG. 5. This reaction is based on previously reported diboron-catalyzed dehydrative amidation reactions.⁶¹ The diboron-catalyzed conjugation reaction showed promising results for proteins. High-performance liquid chromatography (HPLC) chromatogram analysis of a reaction involving an exemplary protein substrate/starting material, i.e., bivalirudin, showed significant conversion of starting material to bioconjugate when the protein substrate was contacted with an alkoxyamine (e.g., as an exemplary amine starting material) in the presence of a diboron compound in an ionic liquid solvent.

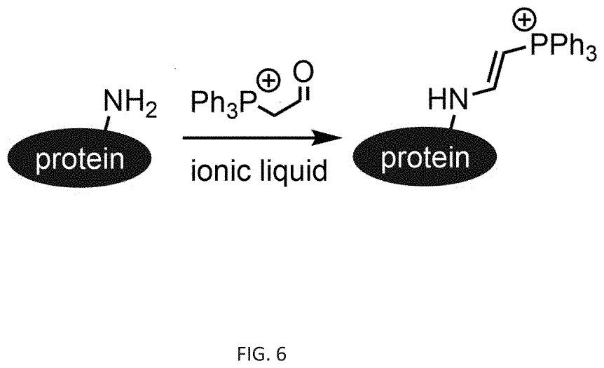

Further, bioconjugation reactions involving aminoalkyl moiety-containing biomolecules substrates (e.g., proteins or peptides) and triarylphosphonium aldehydes can be performed in ionic liquid solvents to provide a bioconjugate comprising a enamine. See FIG. 6. The same reaction was not observed to take place in aqueous solution. Without being bound to any one theory, this may be because, in water, the exemplary triarylphosphonium aldehyde P-(formylmethyl)-triphenylphosphonium has been reported to have low reactivity due to the strong covalent hydration of the carbony group.⁶² The enamine-phosphonium group is stable, even in aqueous solution.

Compounds containing a triarylphosphonium group available via this bioconjugation reaction have interest in a variety of applications, including, for example, the development of antioxidants and anticancer drugs and as functional probes in the mitochondria.⁶³

### III. Bioconjugation in Ionic Liquids

Accordingly, in some embodiments, the presently disclosed subject matter provides a method of preparing a bioconjugate wherein the method comprises performing a bioconjugation reaction in a reaction mixture comprising an ionic liquid. For instance, the reaction mixture can comprise a solvent or solvent mixture comprising one or more ionic liquids. In some embodiments, the solvent or solvent mixture consists essentially of one or more ionic liquids. In some embodiments, the solvent or solvent mixture (and the reaction mixture as a whole) comprises less than about 6% by volume of water (e.g., less than about 6%, 5%, 4%, 3%, 2%, or 1% water).

In some embodiments, the presently disclosed subject matter provides a method of performing a chemoselective bioconjugation reaction, i.e., wherein only a certain chemical functional groups on a biomolecule substrate form a linkage (e.g., a covalent linkage) to another reactant during the bioconjugation reaction. In some embodiments, the method comprises contacting a biomolecule substrate with a functionalized molecule, wherein said functionalized molecule comprises at least one chemical functional group that can form a bond with a chemical functional group present in said biomolecule substrate, and wherein the contacting is performed in a reaction mixture comprising a solvent or solvent mixture comprising, consisting essentially of, or consisting of, an ionic liquid, thereby forming a bioconjugate product.

Any suitable biomolecule substrate can be used. In some embodiments, the biomolecule substrate comprises or has been functionalized to comprise at least one functional group selected from a primary aminoalkyl group, a secondary aminoalkyl group, a carboxylic acid group, and an azide. In some embodiments, the biomolecule substrate comprises a peptide, a protein, a nucleic acid, a carbohydrate, or a lipid.

In some embodiments, when the biomolecule substrate is a carbohydrate, the biomolecule substrate is a carbohydrate other than a cellulose or a lignocellulose. In some embodiments, the biomolecule substrate is a carbohydrate that comprises an aminosugar. In some embodiments, the biomolecule substrate is aminosaccharide. In some embodiments, the biomolecule substrate is an aminosaccharide other than chitosan or chitan. In some embodiments, the biomolecule substrate is a GAG or a glycopeptide. In some embodiments, the carbohydrate biomolecule substrate is selected from hyaluronic acid, dermatan sulfate, chondroitin sulfate, heprin, heparan sulfate, and keratan sulfate. In some embodiments, the biomolecule substrate is a therapeutic aminosaccharide (e.g., a therapeutic glycopeptide). In some embodiments, the therapeutic aminosaccharide is an anti-cancer drug (e.g., doxorubicin or valrubicin) or an antibiotic (e.g., vancomycin or oritavancin).

In some embodiments, the biomolecule substrate is a peptide, a protein, or a nucleic acid (e.g., a DNA, RNA, aptamer, etc.). Exemplary peptide, protein, and nucleic acid biomolecule substrates include, but are not limited to, an enzyme, an antigenic protein, a chemokine, a cytokine, a cellular receptor, a cellular receptor ligand, an aptamer, and an antibody or active fragment thereof.

In some embodiments, the biomolecule substrate (e.g., the peptide or protein biomolecule substrate) has a molecular weight of greater than about 1 kilodalton (kDa). In some embodiments, the biomolecule substrate has a molecular weight of greater than about 25 kDa, about 50 kDa, about 100 kDa, 150 kDa, 200 kDa, about 250 kDa, about 500 kDa. or about 1,000 kDa. In some embodiments, the biomolecule substrate has a molecular weight greater than about 2,000 kDa. In some embodiments, the biomolecule substrate has a molecular weight of about 1 kDa to about 150 kDa.

The solvent or solvent mixture can comprise any suitable ionic liquid or a mixture of ionic liquids. In some embodiments, the ionic liquid comprises a cation selected from the group comprising imidazolium, alkyl-imidazole, alkyl-ammonium, alkyl-sulfonium, alkyl-piperidinium, alkyl-pyridinium, alkyl-phosphonium, and alkyl-pyrrolidinium. In some embodiments, the ionic liquid comprises an anion selected from the group consisting of carboxylate, halide, fulminate, persulfate, sulfate, sulfites, phosphates, phosphites, nitrate, nitrites, hypochlorite, chlorite, bicarbonates, imides, sulfonimides, and borates. In some embodiments, the ionic liquid comprises one or more of the group comprising 1-butyl-1-methylpyrrolidinium trifluoromethanesulfonate (BMPy OTf), 1-butyl-1-methylpyrrolidinium bis(trifluoromethanesulfonyl)imide (BMPy NTf₂), 1-ethyl-3-methylimidazolium acetate (EMIM OAc), 1-butyl-3-methylimidazolium acetate (BMIM OAc), 1-butyl-3-methylimidazolium trifluoromethanesulfonate (BMIM OTf), 1-butyl-3-methylimidazolium bis(trifluoromethanesulfonyl)imide (BMIM NTf₂), 1-butyl-3-methylimidazolium tetrafluoroborate (BMIM BF₄), and tributylethylphosphonium diethylphosphate (TBEP). In some embodiments, the ionic liquid comprises or consists of BMP OTf. In some embodiments, the ionic liquid comprises or consists of EMIM OAc.

As noted hereinabove, generally, the solvent or solvent mixture (or reaction mixture as a whole) comprises minimal water or aqueous solution (e.g., less than 6% by volume water). However, in some embodiments, organic solvents can be included in the reaction mixture as cosolvents. Thus, in some embodiments, the solvent or solvent mixture comprises one or more ionic liquids and one or more polar aprotic organic solvents, such as, but not limited to, dimethylsulfoxide (DMSO), dimethylformamide (DMF), tetrahydrofuran (THF), ethyl acetate, and acetonitrile (ACN or MeCN). In some embodiments, the solvent or solvent mixture can comprise one or more ionic liquids and one or more polar, aprotic organic solvents where the ratio of ionic liquid(s) to polar aprotic solvent(s) is about 1:0.01 to about 1:1 (e.g., about 1:0.01, 1:0.1, 1:0.2, 1:0.3, 1:0.4, 1:0.5, 1:0.6, 1:0.7, 1:0.8, 1:0.9, or about 1:1). Thus, typically, the solvent or solvent mixture comprises at least about 50% ionic liquid.

In some embodiments, one of the two “starting materials” of the bioconjugation reaction (i.e., one of the biomolecule substrate and the functionalized molecule) comprises an aminoalkyl group and the other comprises an azide group. In some embodiments, the biomolecule substrate comprises one or more aminoalkyl moiety (e.g., one or more primary aminoalkyl and/or secondary aminoalkyl moiety), the functionalized molecule is an azide-containing compound (e.g., a molecule functionalized to contain an azide group), and the reaction mixture further comprises a phosphine. In some embodiments, the phosphine is a triarylphosphine. In some embodiments, each aryl group of the triaryl phosphine is independently a substituted or unsubstituted phenyl or pyridyl group. Exemplary substituents for the phenyl and pyridyl groups of the triarylphosphine include, but are not limited to, alkyl (e.g., C₁-C₆ alkyl), alkoxy, carboxy, and sulfonate. In some embodiments, the triarylphosphine is selected from the group comprising triphenylphosphine, tri-p-tolylphosphine, tris(4-methoxyphenyl)phosphine; diphenyl(m-sulfonatophenyl)phosphine or a salt thereof, and 4-(diphenylphosphino)benzoic acid. In some embodiments, the bioconjugate product of the bioconjugation between the aminoalkyl moiety-containing molecule and the azide-containing molecule comprises a urea linkage. For instance, the urea linkage can covalently link a monovalent derivative of biological substrate to a monovalent derivative of the functionalized molecule.

In some embodiments, the biological substrate is a peptide or a protein and the aminoalkyl moiety comprises an amino group of a terminal amino acid residue in the protein or peptide or is an amino group of a lysine residue side chain in the peptide or protein. In some embodiments, the peptide or protein is an antibody or antibody fragment. In some embodiments, the biomolecule substrate comprises more than one aminoalkyl moiety. In some embodiments, the biomolecule substrate (e.g., the peptide or protein biomolecule substrate) comprises between 2 and 10 aminoalkyl moieties (i.e., 2, 3, 4, 5, 6, 7, 8, 9, or 10 aminoalkyl moieties). In some embodiments, the biomolecule substrate (e.g., the peptide or protein biomolecule substrate) comprises more than 10 aminoalkyl moieties. In some embodiments, the bioconjugate product comprises linkages to more than one functionalized molecule, i.e., one from each former aminoalkyl moiety site.

In some embodiments, the biomolecule substrate comprises a nucleic acid (e.g., an oligonucleotide, a single-stranded DNA or a double-stranded DNA) functionalized with a primary aminoalkyl group. For instance, the nucleic acid can be functionalized (e.g., at the 5′ or 3′ end) with one of the modifiers of Scheme 1, below. In some embodiments, the aminoalkyl group comprises a NH₂ group attached to the nucleic acid via a C₆-C₁₂ alkylene group.

The azide-containing compound can be any suitable azide-containing compound. In some embodiments, the azide-containing compound comprises an azide-containing derivative of one of the group comprising a small molecule therapeutic agent; a nucleic acid: a lipid (e.g., cholesterol); a carbohydrate; a polymer; and a detectable label. In some embodiments, the detectable label comprises a dye, fluorophore, an affinity label, or a radioisotope. In some embodiments, the detectable label is biotin or a derivative thereof. In some embodiments, the detectable label is desthiobiotin. In some embodiments, the detectable label is a fluorescent molecule. In some embodiments, the polymer is a hydrophilic polymer, such as, but not limited to polyethylene gycol (PEG) or PVP. Hydrophilic polymers, such as PEGs, present in a protein bioconjugate can reduce the immunogenicity of the protein or increase half-life in vivo.

In view of the chemoselectivity of the presently disclosed amine-azide bioconjugation reaction, the azide-containing compound can comprise a wide variety of additional chemical functional groups that can be present during the bioconjugation reaction and which will not react or interfere with the bioconjugation reaction. For instance, the azide-containing compound can include (in addition to an azide group), one or more of the chemical functional groups including, but not limited to, a carbamate group, a hydroxy group, a thiol, an ether, a tertiary amine, an ester, a halide, an amide group, an imide group, and a urea group.

In some embodiments, the contacting is performed at a temperature of about 20 degrees Celsius (° C.) to about 70° C. (e.g., about 20, 25, 30, 35, 40, 45, 50, 55, 60, 65 or about 70° C.). In some embodiments, the temperature is about 37° C. to about 50° C. In some embodiments, the contacting is performed for about 30 minutes to about 72 hours. In some embodiments, the contacting is performed for about 40 minutes to about 16 hours (e.g., about 40 minutes, 1 hour, 2 hours, 4 hours, 6 hours, 8 hours, 10 hours, 12 hours, 14 hours, or 16 hours). In some embodiments, the contacting is performed for about 40 minutes to about 4 hours (e.g., about 40 minutes, 60 minutes, 100 minutes, 120 minutes, 140 minutes, 160 minutes, 200 minutes, or about 240 minutes).

In some embodiments, the reaction mixture can comprise a molar excess of the azide-containing compound and/or phosphine (e.g., triarylphosphine) as compared to the aminoalkyl-containing biomolecule substrate. In some embodiments, the biomolecule substrate is present in the reaction mixture at a concentration of about 0.025 millimolar (mM) to about 0.5 mM; the azide-containing compound is present at a concentration of about 0.3 mM to about 125 mM, and the phosphine is present at a concentration of about 3 mM to about 125 mM. In some embodiments, the azide-containing compound and/or the phosphine are present at a concentration of about 3 mM to about 20 mM. In some embodiments, the azide-containing compound and/or the phosphine are present at a concentration of about 3 mM to about 7.5 mM. In some embodiments, the biomolecule substrate is present at a concentration of about 0.1 mM to about 0.4 mM.

In some embodiments, the reaction mixture further comprises a buffer, e.g., a bicarbonate buffer, a borate buffer, or an acetate buffer. In some embodiments, the buffer comprises an alkali bicarbonate salt. In some embodiments, the alkali bicarbonate salt is sodium bicarbonate (NaHCO₃) or potassium bicarbonate (KHCO₃). In some embodiments, the buffer comprises KHCO₃. In some embodiments, the alkali bicarbonate salt is present in the reaction mixture at a concentration of about 20 mM.

In some embodiments, the method further comprises isolating the bioconjugate product. For instance, in some embodiments, the method further comprises extracting the bioconjugation product into an aqueous liquid. In some embodiments, the method comprises precipitating the bioconjugation product. In some embodiments, the method comprises purifying the bioconjugation product, e.g., via liquid chromatography (e.g., HPLC) or some other suitable technique as would be apparent to one of ordinary skill in the art.

The ionic liquid bioconjugation reactions are not limited to azide-amine reactions, but also include conjugation reactions between amino groups and carboxylic acid groups and conjugation reactions between amino groups and triarylphosphonium aldehydes. Thus, in some embodiments, the biomolecule substrate comprises a protein comprising one or more carboxylic acid group; the functionalized molecule comprises an amino group (e.g., an alkoxyamine); the reaction mixture further comprises a diboron compound (e.g., tetrahydroxyl diboron); and the bioconjugation product comprises an amide linkage. Exemplary reaction mixtures for the diboron-mediated bioconjugation reaction include reaction mixtures with the following concentrations of materials in an ionic liquid solvent: about 0.05 mM to about 0.3 mM biomolecule substrate; about 5 mM to about 100 mM amine (e.g., alkoxyamine); and about 5 mM to about 100 mM diboron compound. In some embodiments, the reaction can be performed in BMPy OTf at 50° C. for several hours (e.g., overnight). In some embodiments, the reaction is performed at a temperature at which the solvent or solvent mixture refluxes. In some embodiments, the reaction is performed in the presence of air.

In some embodiments, the biomolecule substrate comprises a protein or peptide comprising one or more aminoalkyl group; the functionalized molecule comprises a triarylphosphonium aldehyde; and the bioconjugation product comprises an enamine linkage. In some embodiments, the triarylphosphonium aldehyde is a triphenylphosphonium aldehyde. In some embodiments, the triphenylphosphonium aldehyde is a (formylmethyl)triphenylphosphonium salt (e.g., (formylmethyl)triphenylphosphonium chloride). Exemplary reaction mixtures for the bioconjugation reaction to form enamine linkages include mixtures with the following concentrations of materials: about 0.1 M peptide/protein; about 10 mM triarylphosphonium aldehyde; and about 20 mM K₂CO₃ in about 40 microliters (μl) BMPy OTf.

In some embodiments, the presently disclosed subject matter provides a bioconjugate prepared according to a bioconjugation reaction as described herein. For instance, in some embodiments, the presently disclosed subject matter provides a bioconjugate comprising a urea linkage or an enamine (e.g., a triarylphosphonium-substituted enamine). In some embodiments, the bioconjugate comprises a first monovalent derivative and a second monovalent derivative, wherein the first monovalent derivative and the second monovalent derivative are attached to one another via a urea linkage, and wherein the first monovalent derivative is a monovalent derivative of a protein, peptide, aminosaccharide, or nucleic acid, and the second monovalent derivative is a monovalent derivative of a small molecule therapeutic agent; a nucleic acid; a lipid (e.g., cholesterol): a carbohydrate; a polymer; and a detectable label. In some embodiments, the bioconjugate is an antibody-drug conjugate (ADC).

### IV. Amine-Azide Bioconjugation in Ionic Liquids

In some embodiments, the presently disclosed subject matter provides a method of performing a chemoselective bioconjugation reaction between an aminoalkyl-containing molecule and an azide-containing molecule in an ionic liquid. In some embodiments, the method comprises contacting a first molecule and a second molecule in a reaction mixture comprising a triarylphosphine and a solvent or solvent mixture, wherein said first molecule comprises an aminoalkyl group (e.g., a primary aminoalkyl group or a secondary aminoalkyl group), wherein said second molecule comprises an azide group, and wherein the solvent or solvent mixture comprises, consists essentially of, or consists of an ionic liquid, thereby forming a bioconjugate product comprising a urea linkage, and wherein at least one of said first molecule and said second molecule comprises a biomolecule or a derivative thereof. In some embodiments, the biomolecule or derivative thereof is selected from a protein, a peptide, a nucleic acid, a carbohydrate (e.g., an aminosaccharide), and derivatives thereof.

In some embodiments, the ionic liquid comprises a cation selected from the group including, but not limited to, imidazolium, alkyl-imidazole, alkyl-ammonium, alkyl-sulfonium, alkyl-piperidinium, alkyl-pyridinium, alkyl-phosphonium, and alkyl-pyrrolidinium. In some embodiments, the ionic liquid comprises an anion selected from the group including, but not limited to, carboxylate, halide, fulminate, persulfate, sulfate, sulfites, phosphates, phosphites, nitrate, nitrites, hypochlorite, chlorite, bicarbonates, imides, sulfonimides, and borates. In some embodiments, the ionic liquid comprises one or more of the group comprising BMPy OTf, 1-butyl-1-BMPy NTf₂, EMIM OAc, BMIM OAc, BMIM OTf, BMIM NTf₂, BMIM BF₄, and TBEP. In some embodiments, the ionic liquid comprises or consists of BMP OTf. In some embodiments, the ionic liquid comprises or consists of EMIM OAc.

In some embodiments, the solvent or solvent mixture comprises one or more ionic liquids and one or more polar aprotic organic solvents, such as, but not limited to, DMSO, DMF, THF, ethyl acetate and ACN. In some embodiments, the solvent or solvent mixture can comprise one or more ionic liquids and one or more polar, aprotic organic solvents where the ratio of ionic liquid(s) to polar aprotic solvent(s) is about 1:0.01 to about 1:1 (e.g., about 1:0.01, 1:0.1, 1:0.2, 1:0.3, 1:0.4, 1:0.5, 1:0.6, 1:0.7, 1:0.8, 1:0.9, or about 1:1). Thus, typically, the solvent or solvent mixture comprises at least about 50% ionic liquid. In some embodiments, the solvent or solvent mixture (or reaction mixture) comprises less than about 6% water by volume.

In some embodiments, the first molecule is a peptide, protein, nucleic acid or aminosaccharide. In some embodiments, the second molecule is an azide containing-saccharide. In some embodiments, the second molecule is an azide-containing derivative of one of the group comprising a small molecule therapeutic agent; a nucleic acid; a lipid (e.g., cholesterol); a carbohydrate: a polymer (e.g., a hydrophilic polymer); and a detectable label (e.g., a dye, a fluorophore, an affinity label, or a radioisotope). In addition to the azide and aminoalkyl group, the first and second molecules can include a variety of other chemical functional groups that will not react or interfere with the bioconjugation reaction. These other groups include, but are not limited to, a carbamate group, a hydroxy group, a thiol, an ether, a tertiary amine, an ester, a halide, an amide group, an imide group, and a urea group.

The triarylphosphine can be any suitable triarylphosphine as described hereinabove. In some embodiments, the triarylphosphine is selected from the group consisting of triphenylphosphine, tri-p-tolylphosphine, tris(4-methoxyphenyl)phosphine: diphenyl(m-sulfonatophenyl)phosphine or a salt thereof, and 4-(diphenylphosphino)benzoic acid.

In some embodiments, the first molecule is present in the reaction mixture at a concentration of about 0.025 mM to about 0.5 mM: the second molecule is present at a concentration of about 0.3 mM to about 125 mM; and the triarylphosphine is present at a concentration of about 3 mM to about 125 mM. In some embodiments, the second molecule and/or the triarylphosphine are present at a concentration of about 3 mM to about 20 mM. In some embodiments, the second molecule and/or the triarylphosphine are present at a concentration of about 3 mM to about 7.5 mM. In some embodiments, the first molecule is present at a concentration of about 0.1 mM to about 0.4 mM.

In some embodiments, the contacting is performed at a temperature of about 20 degrees Celsius (° C.) to about 70° C. (e.g., about 20, 25, 30, 35, 40, 45, 50, 55, 60, 65 or about 70° C.). In some embodiments, the temperature is about 37° C. to about 50° C. In some embodiments, the contacting is performed for about 30 minutes to about 72 hours. In some embodiments, the contacting is performed for about 40 minutes to about 16 hours (e.g., about 40 minutes, 1 hour, 2 hours, 4 hours, 6 hours, 8 hours, 10 hours, 12 hours, 14 hours, or 16 hours). In some embodiments, the contacting is performed for about 40 minutes to about 4 hours (e.g., about 40 minutes, 60 minutes, 100 minutes, 120 minutes, 140 minutes, 160 minutes, 200 minutes, or about 240 minutes).

In some embodiments, the reaction mixture further comprises a buffer, e.g., a bicarbonate buffer, a borate buffer, or an acetate buffer. In some embodiments, the buffer comprises an alkali bicarbonate salt. In some embodiments, the alkali bicarbonate salt is NaHCO₃ or KHCO₃. In some embodiments, the buffer comprises KHCO₃. In some embodiments, the alkali bicarbonate salt is present in the reaction mixture at a concentration of about 20 mM.

In some embodiments, the method further comprises isolating the bioconjugate product. For instance, isolation can be performed via liquid-liquid extraction. More particularly, when the bioconjugation product comprises a conjugate of a hydrophilic biomolecule and the ionic liquid is water immiscible (e.g., BMPy NTf₂ or another bistriflimide-containing ionic liquid), the bioconjugation product can be extracted into an aqueous liquid. If the reaction is performed in a water miscible ionic liquid (e.g., BMPy OTf), a water immiscible ionic liquid can be added to the reaction mixture prior to extraction to enhance the extraction of the bioconjugate product into water or an aqueous solution. When the bioconjugate product comprises a conjugate of a more hydrophobic biomolecule (e.g., a hydrophobic saccharide), the reaction can be performed in a ionic liquid that is less soluble in organic solvents (e.g., EMIM OAc) and the bioconjugate product can be extracted into an organic solvent (e.g., ethyl acetate). In some embodiments, e.g., when the bioconjugate product is hydrophilic, the bioconjugate product can be isolated by precipitating it with an organic solvent (e.g., acetone). In some embodiments, the method comprises purifying the bioconjugation product, e.g., via liquid chromatography (e.g., HPLC) or some other suitable technique as would be apparent to one of ordinary skill in the art.

In some embodiments, the presently disclosed subject matter provides a bioconjugate prepared according the presently disclosed amine-azide bioconjugate reaction.

### EXAMPLES

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter.

### Materials and Reagents

All the chemicals including peptides and proteins were purchased from commercial vendors unless otherwise noted. A list of peptides/proteins and azide compounds used in the examples is provided in Tables 3 and 4, respectively, hereinbelow. Peptides and proteins were purchased from commercial sources as shown in Table 3: MedChemExpress (Monmouth Junction, N.J. United States of America), Sigma-Aldrich (St. Louis, Mo., United States of America), Bachem Holding AG (Bubendorf, Switzerland). APExBio (Houston, Tex., United States of America), Gbiosciences (St. Louis, Mo., United States of America), Jacksonimmuno (West Grove, Pa., United States of America), and Biosynth Carbosynth (Staad, Switzerland). Azides in Table 4, with the exception of 2 and S7, were purchased from Combi-Blocks (San Diego, Calif., United States of America), Sigma-Aldrich (St. Louis. Mo., United States of America), TCI (Tokyo, Japan) and Chem-Impex (Chem-Impex International, Wood Dale, Ill., United States of America). 1-Azido-2-(2-methoxyethoxy)ethane 2⁵⁷ and Boc-protected SN-38⁵⁸ were synthesized according to previous reports. ¹⁵N-enriched sodium azide was purchased from Millipore-Sigma (#609374: Burlington, Mass., United States of America). 1-Butyl-1-MethylPyrrolidinium triflate (BMPy OTf) was purchased from Tokyo Chemical Industry (B5568; Tokyo, Japan) or Synthonix (B52266; Wake Forest, N.C., United States of America). Molecular weight marker was purchased from Thermo Scientific (#26619; Waltham, Mass., United States of America).

TABLE 3
Peptides and Proteins.

| | SEQ ID | | | | |
|---|---|---|---|---|---|
| | NO or | | | | |
| Name (concn^{a}; | Uniprot | MW | Azide | Phosphine | |
| supplier, catalog #) | ID | (Da) | (concn)^{a} | concn^{a} | |
| Angiotensin II | 1 | 1046.2 | S4 (7.5 mM) | 20 | mM |
| (0.2 mM)^{b}, | | | | | |
| MedChemExpress, | | | | | |
| HY-13948) | | | | | |
| α-MSH (0.3 mM, | 2 | 1664.9 | 1 (7.5 mM) | 20 | mM |
| Sigma-Aldrich, M4125) | | | | | |
| OGP (0.3 mM, | 3 | 1523.7 | S4 (7.5 mM) | 20 | mM |
| MedChemExpress, | | | | | |
| HY-P1563) | | | | | |
| Substance P (0.4 mM, | 4 | 1347.6 | 2 (7.5 mM) | 20 | mM |
| Bachem, H-1890) | | | | | |
| LHRH (0.2 mM, | 5 | 1182.3 | S4 (7.5 mM) | 20 | mM |
| Sigma-Aldrich, L7134) | | | | | |
| Daptomycin (0.1 mM, | 6 | 1619.7 | 2 (7.5 mM) | 20 | mM |
| APExBio, A1206) | | | | | |
| Enfuvirtide (75 μM, | 7 | 4491.9 | 2 (7.5 mM) | 3 | mM |
| MedChemExpress, HY- | | | | | |
| P00524) | | | | | |
| Lysozyme (25 μM, | P00698 | 14k | S7 (3 mM) | 3 | mM |
| Sigma-Aldrich, L4919) | | | | | |
| α-chymotrypsinogen A | P00766 | 16k | S7 (3 mM) | 3 | mM |
| (25 μM, Sigma-Aldrich, | | | | | |
| C4879) | | | | | |
| Streptavidin (75 μM, | P22629 | 53k | S5 (7.5 mM) | 7.5 | mM |
| Gbiosciences, 786-584) | | | | | |
| Polyclonal anti-Ms | | 50k | 3 (7.5 mM) | 3 | mM |
| antibody Fab | | | | | |
| fragment (7.5 μM, | | | | | |
| Jacksonimmuno | | | | | |
| (315-007-003) | | | | | |
| Herceptin (10 μM, | | 150k | S7 (3 mM) | 3 | mM |
| Biosynth Carbosynth, | | | | | |
| FT65040) | | | | | |

^{a}Representative concentration (concn) of reagents for the modification reaction.
^{b}Reaction time was elongated to 4 hours.

TABLE 4
Azide Compounds for Peptide and Protein Bioconjugation Studies.

| | Compound | | Supplier |
|---|---|---|---|
| Structure | # | Groups | (Catalog #) |
| 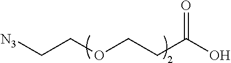 | 1 | Carboxylate | Combi- Blocks (QA-4033) |
| 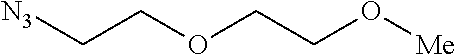 | 2 | MeO | synthesized |
| 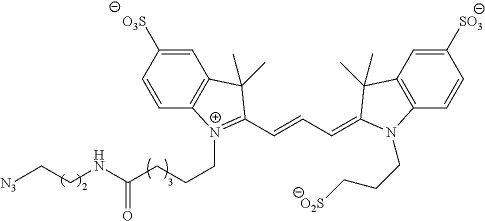 | 3″ | Cy3 | Sigma-Aldrich (777315) |
| 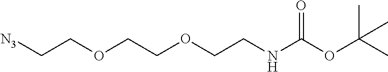 [SMILES: CC(C)(C)OC(=O)NCCOCCOCCN=[N+]=[N-]] | S3 | Boc | Combi- Blocks (QJ-6227) |
| 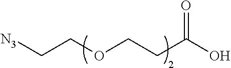 | S4 | Carboxylate | Combi- Blocks (QJ-7530) |
| 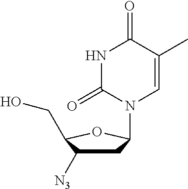 | S5 | Thymidine | TCI (A20521G) |
| 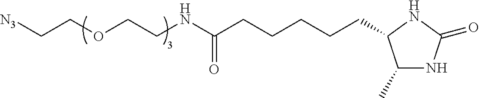 | S6 | Desthiobiotin | Chem-Impex (35116) |
| 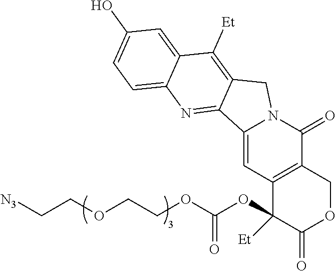 | S7 | SN-38 | synthesized |
| 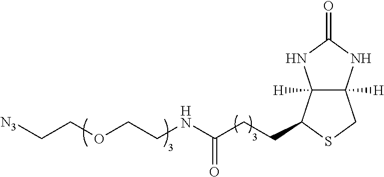 | S8 | Biotin | Sigma- Aldrich (762024) |

SK-BR-3 cells were cultured in Dulbecco's Modified Eagle Medium (DMEM) with Glutamax and 10% fetal bovine serum (FBS) using 24-well cell culture plate (Corning 3524) coated with poly-L-lysine under 5% CO₂ at 37° C. Cells were fixed with 4% paraformaldehyde at 70% confluency, washed with PBS three times, and used for the immunofluorescence studies.

NMR was performed on Bruker AVANCE NEO 600 and 700 (Bruker, Billerica, Mass. United States of America). For ¹⁵N NMR, neat nitromethane (381.6 ppm, TCI N0209 (Tokyo Chemical Industry, Tokyo. Japan) was used as the external standard.

MALDI-MS was conducted on a Bruker Daltonics Autoflex-TOF (Bruker, Billerica, Mass., United States of America. A sample (0.5 or 1 μL) was mixed with an equal volume (0.5 or 1 μL) of matrix solution (20 mg/mL soln in 50:50:0.1 H₂O/MeCN/trifluoroacetic acid) on a ground-steel MALDI plate (Bruker 8280784, Billerica, Mass., United States of America). Sinapic acid or α-cyano-4-hydroxycinnamic acid (CHCA) was used as a matrix.

Tandem mass spectrometry was performed on an Orbitrap Elite (Thermo Scientific, Waltham, Mass. United States of America) mass spectrometer for undigested peptide samples. Each peptide was dissolved at 10 pmol/μL in 0.1% formic acid in water and analyzed by direct injection at 40 uL/min. Approximately 0.08 ug of the modified peptide sample was used for the analysis. The Orbitrap Elite, coupled to nano-flow HPLC (Agilent Technologies, Santa Clara, Calif., United States of America) was used for LC/MS/MS analysis of digested protein samples. Digestion of the protein sample was performed by incubation of the protein sample (4.5 ug) with 100 uL washed immobilized pepsin beads (Thermo Scientific. Waltham, Mass., United States of America) at 50% slurry in 200 mM ammonium acetate in water, pH 2.3 at 4° C. for 5 mm.

LC-MS analysis of peptide reactions (substance P and daptomycin) and were performed on Shimadzu LCMS-2020 (Shimadzu, Kyoto, Japan) with a 2.6 μm C18 column (50×2.1 mm). The flow rate was 1 mL/min with the gradient of acetonitrile (5-90%) in the presence of 0.1% formic acid. The analysis of the reactions were performed by the UV detection of peptide peaks at 280 nm. Benzoylarginine ethyl ester (0.5 mM) was added to each sample as an internal standard.

For all the other peptides, LC-MS analysis was performed on Agilent Technologies 1260 Infinity II series single quad instrument with 5 μm Luna C18 column (150×4.6 mm) (Agilent Technologies, Santa Clara, Calif., United States of America). The flow rate was 0.5 mL/min with the gradient of acetonitrile (10-90%) in the presence of 0.1% trifluoroacetic acid. The analysis of the reactions were performed by the UV detection of peptide peaks at 280 nm. 1,3,5-trimethoxybenzene (0.5 mM) was added to each sample as an internal standard.

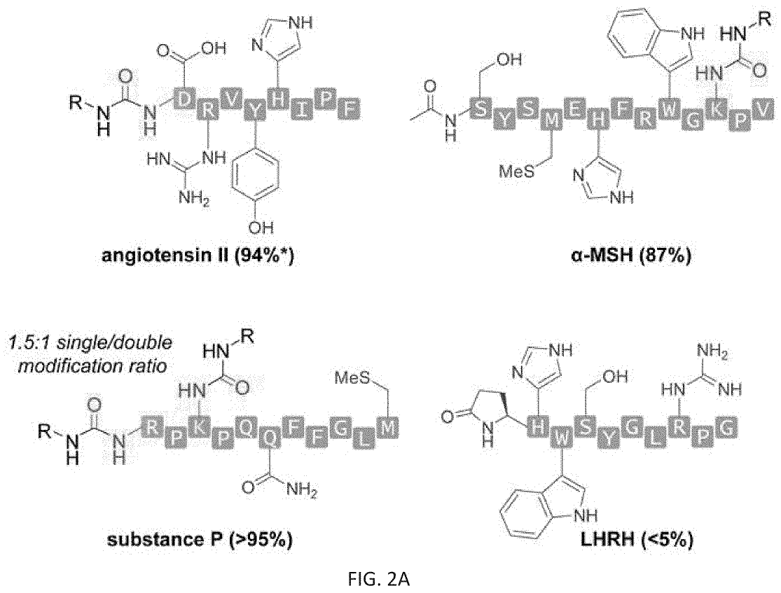

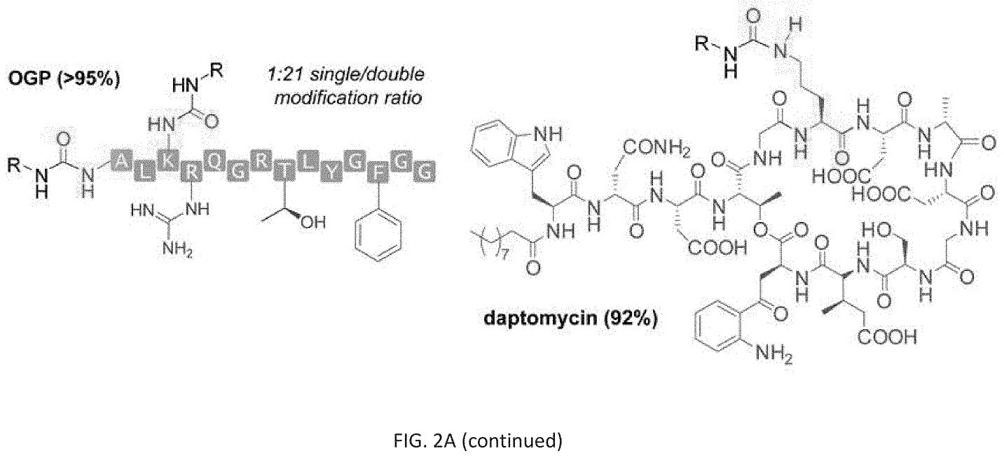

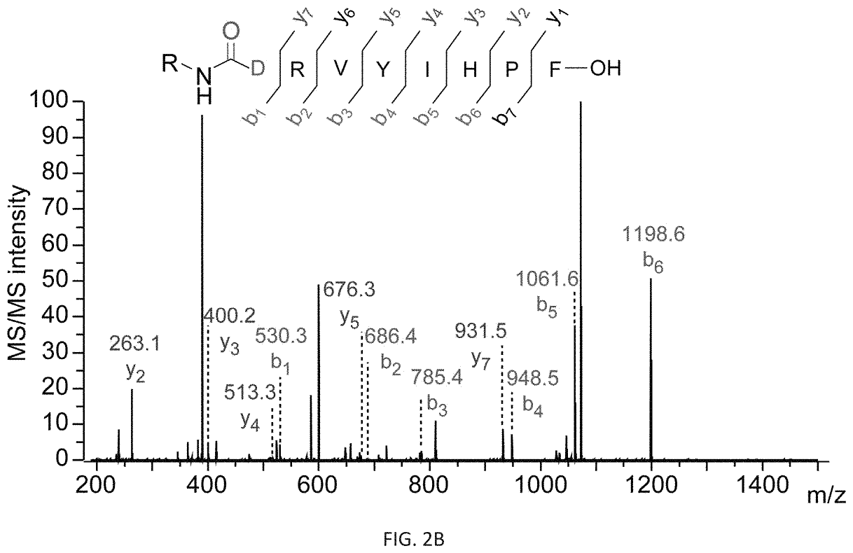

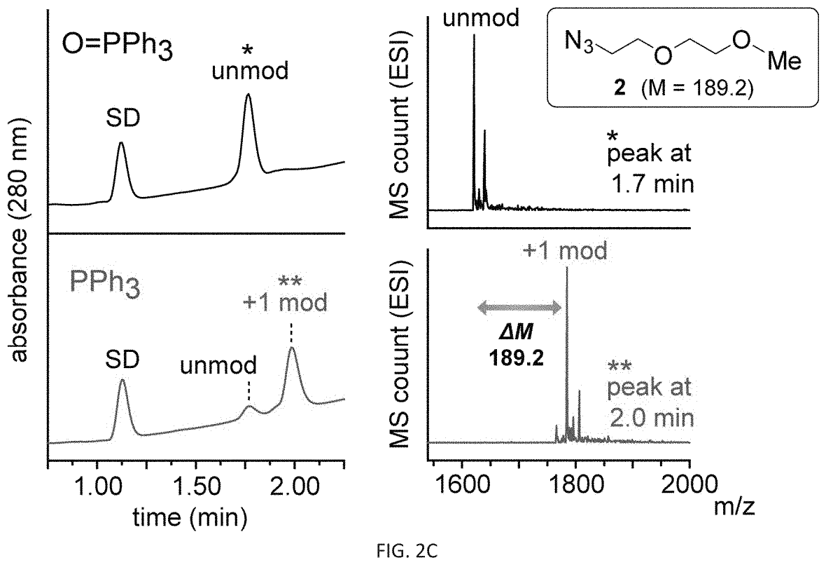

The conversions shown in FIG. 2A were calculated by dividing the product peak area by the sum of product peak area and starting material peak area. The LC-MS yield was calculated using a calibration curve.

Gel fluorescence and western blot imaging was conducted on Amersham IMAGEQUANT® 800 (Cytiva, Upsala, Sweden). Gel or blot fluorescence imaging were performed using 360-nm (SN-38), 535-nm (Cy3), and 635-nm (Cy5) light sources with corresponding emission bandpass filters at 525 nm (±20 nm), 605 nm (±40 nm), and 705 (±40 nm), respectively. Anti-biotin western blot was performed with either streptavidin-HRP conjugate (Jackson ImmunoResearch (West Grove, Pa., United States of America), 016-030-084, 1:10,000 dilution) or streptavidin-Cy5 conjugate (Jackson ImmunoResearch (West Grove, Pa., United States of America), 016-170-084, 1:2,000 dilution for western blot and 1:50 for immunofluorescence) after blocking with 5% BSA in TBST buffer.

Confocal microscope: Fluorescence microscope imaging was performed on Zeiss laser scanning microscope 710 (Carl Zeiss AG, Oberkochen, Germany) with a 40× water-immersion C-Apochromat objective lens (numerical aperture 1.1). Excitation at 405 nm (DAPI), 488 nm (Cy3), and 633 nm (Cy5) were used with filter settings 410-480 nm, 494-631 nm, and 638-759 nm, respectively. Image J software was used to generate images suitable for publication.

UV-vis spectroscopy was performed on UV NANODROP® (Thermo Fisher Scientific, Waltham, Mass., United States of America).

### Example 1

### Typical Peptide Protein Modification Procedure in Ionic Liquid

To BMPy OTf (typically 10-40 μL for analytical scale), potassium bicarbonate aqueous solution (20 mM final concentration (concn) from 2-M stock solution), aqueous solution of peptide/protein (0.025-0.4 mM final concn from 0.55 mM stock solution in H₂O or 50-mM pH 7.4 MES buffer), alkylazide (3-20 mM final concn from 100-500-mM stock solution in DMSO), and PPh₃ or O═PPh₃ (3-7.5 mM final concn from 150-500-mM stock solution in DMSO) were added. The final concn of H₂O was kept lower than 6% v/v. The reaction mixture was incubated at a 37- or 50-° C. incubator for 2 h and subjected to Post-reaction cleanup process for analytical scale reaction (Example 2) before analysis.

### Example 2

### Post-Reaction Cleanup Process for Analytical Scale Reaction

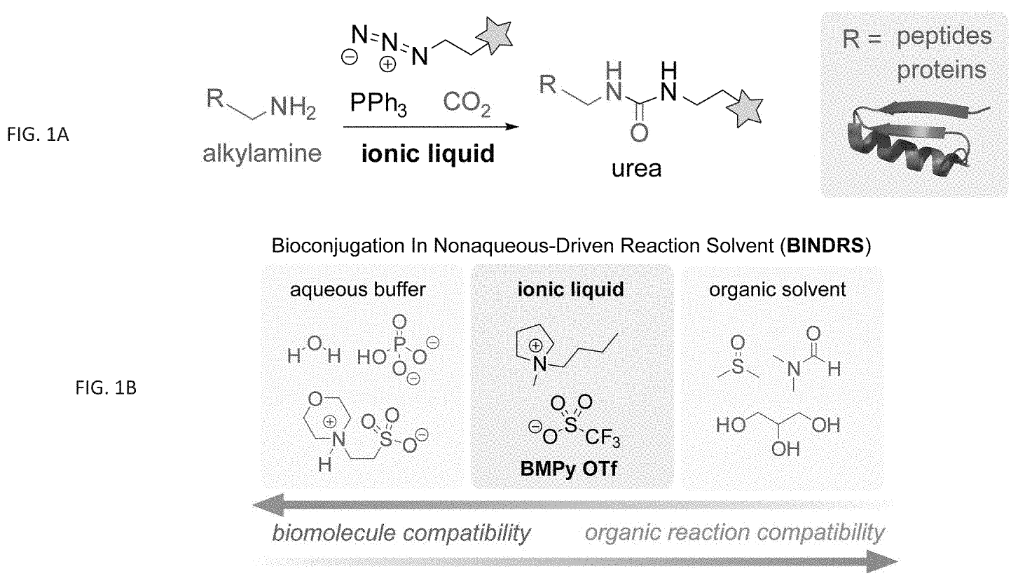

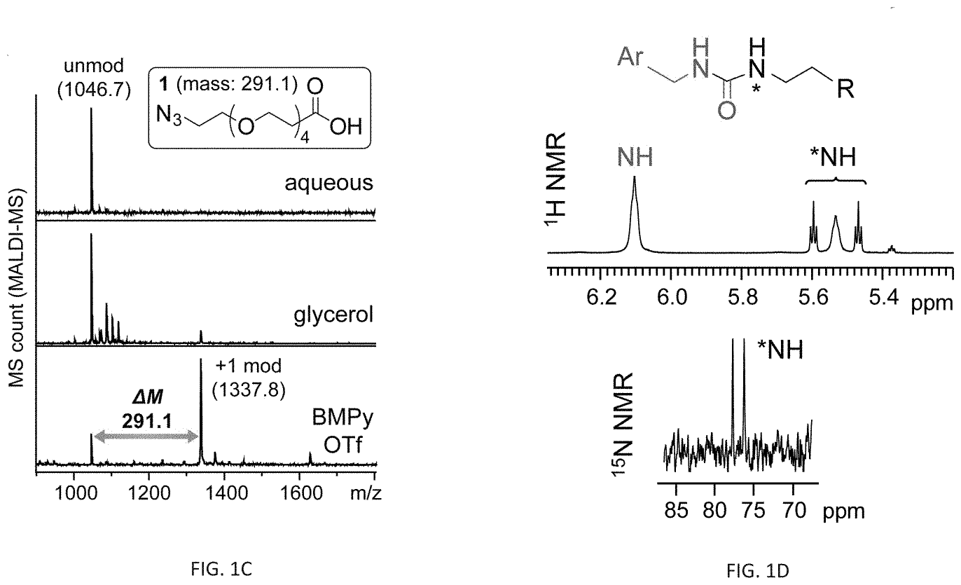

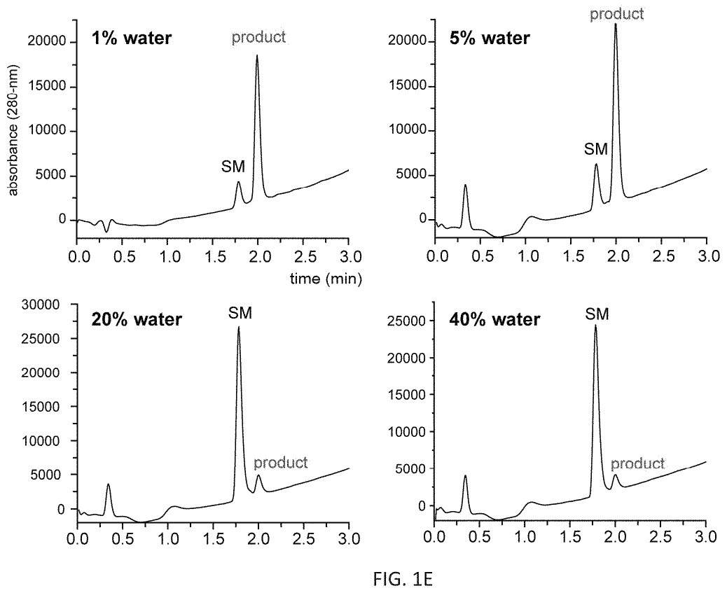

To the reaction mixture (typically 10-40 μL) in a 1.7-mL Eppendorf tube, cold acetone (600-900 μL, −20° C.) was added in one portion. For α-MSH, OGP, Substance P, and streptavidin, 20% volume of H₂SO₄ aqueous solution (stock concn: 5% v/v) was added (e.g. 2 μL of the H₂SO₄ solution to 10 μL reaction volume) before the addition of acetone. For the reactions in glycerol (experiment in FIG. 1C), isopropanol (100 μL) was added to prevent the phase separation from acetone, and the same treatment was applied for other conditions in different medium to eliminate the experimental bias. After the addition of the acetone, the mixture was mixed by upside-down shaking and sit at −80° C. for 1 h (protein samples) or overnight (peptide samples). The precipitates were collected by centrifugation (15,000 rcf, 15 min, 4° C.), and the pellet was air-dried on bench at rt for 15 min after removing acetone. For MALDI-MS analysis as well as samples with acetone-insoluble azide (i.e. Cy3-azide and SN-38-azide), the pellet was further washed by two additional cycles of acetone or methanol addition and centrifugation before the air-drying process. The dried pellet was reconstituted in 10-40 μL of 5 mM N-methylmorpholine buffer (for protein samples) or water (for peptide samples) and analyzed by suitable analytical methods.

### Example 3

### Preparative Synthesis of Daptomycin Modified with Azide 2

To BMPy OTf, potassium bicarbonate aqueous solution (20 mM final concn from 2-M stock solution), daptomycin (7.5 mM final concn from 150 mM stock solution in DMSO), azide 2 (75 mM final concn from 1000-mM stock solution in DMSO), and PPh₃ (125 mM final concn from 1000-mM stock solution in 1:1 DMSO/toluene) were added (total volume 0.24 mL). The reaction mixture was incubated at a 50-° C. incubator for 2 h and subjected to Post-reaction cleanup process for analytical scale reaction (Example 2) using 1.2 mL cold acetone for the precipitation. Conversion of the modification was ˜40% based on the analytical LC-MS. The crude mixture was purified on preparative HPLC (Agilent Technologies 218 purification system, Agilent Technologies, Santa Clara, Calif., United States of America) with a 10-20 μm C18 column (250×22 mm) sold under the tradename VYDAC® (W.R. Grace & Co., Columbia, Md., United States of America)) to afford daptomycin modified with azide 2 (0.635 μg, 20%). The flow rate of the preparative HPLC was 10 mL/min with the gradient of acetonitrile (50-95%) in the presence of 0.1% trifluoroacetic acid.

### Example 4

### Daptomycin Alkali Hydrolysis

The alkali hydrolysis procedure was adapted from a previous report.⁵⁹ After the modification reaction (10-μL scale reaction) and acetone precipitation described above, the dried pellet of daptomycin modified with azide S4 was reconstituted in LiOH aqueous solution (10 μL, 100 mM). After the solution was incubated at rt for 20 min, formic acid solution (0 μL, 5% v/v) was added to neutralize the solution. Acetone (0.6 mL) was added to the solution, and the precipitation/reconstitution process described in Post-reaction cleanup process for analytical scale reaction (Example 2) was performed without the additional acetone wash. The dried pellet was reconstituted in water and analyzed by LC-MS or MS/MS.

### Example 5

### Preparative Scale Synthesis of α-Chymotrypsinogen A-Biotin Conjugate (for the Linkage Stability Study)

To BMPy OTf (89 μL), potassium carbonate aqueous solution (20 mM final concn from 2-M stock solution), aqueous solution of α-chymotrypsinogen A (0.1 mM final concn from 2 mM stock solution 50-mM pH 7.4 MES buffer), biotin-azide (3 mM final concn from 100-mM stock solution in DMSO), and PPh₃ (20 mM final concn from 1000-mM stock solution in DMSO) were added (100 μL total volume). The reaction was replicated at the same time (4×10 μL). The reaction mixture was incubated at 37° C. for 2 h, and cold acetone (0.6 mL) was added to each reaction. After the solution was kept at −80° C. overnight, the precipitates were collected by centrifugation (15,000 rcf, 15 min, 4° C.), and the pellet was further washed by two additional cycles of acetone addition and centrifugation. The pellet was air-dried on bench at rt for 15 min after removing the final acetone solution. The dried pellet was reconstituted in 50 μL of 5 mM N-methylmorpholine buffer. All the reconstituted solutions were combined (200 μL total), aliquoted, and stored at −80° C. for the future use.

### Example 6

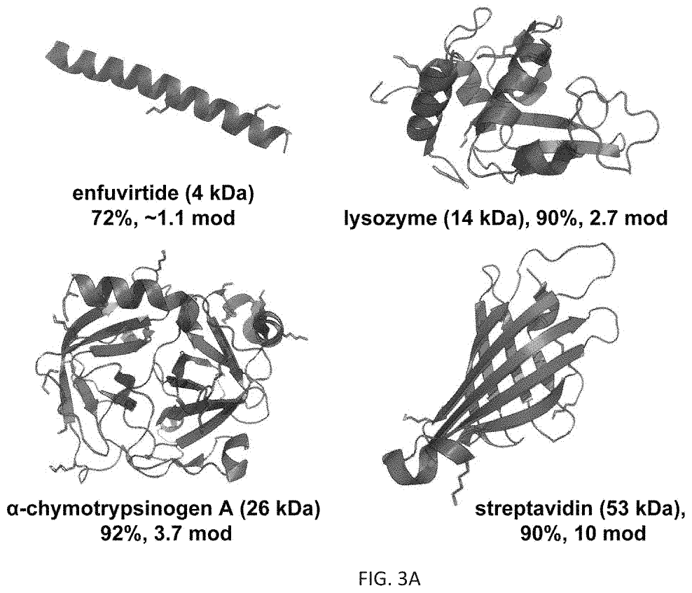

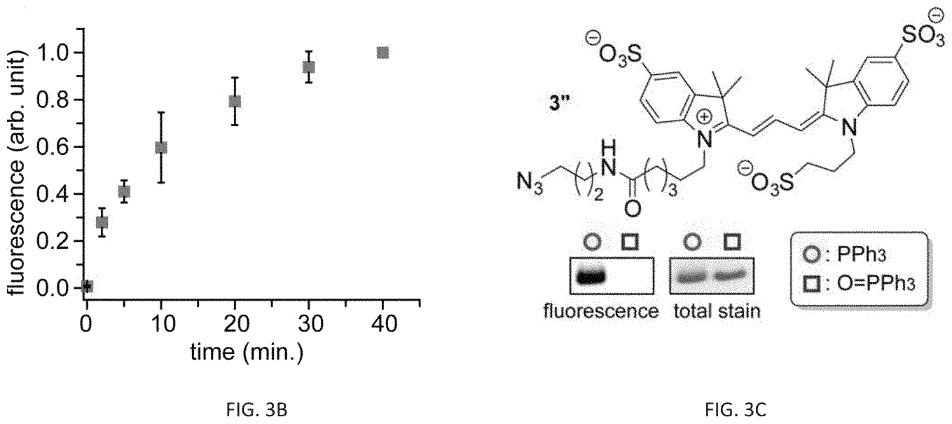

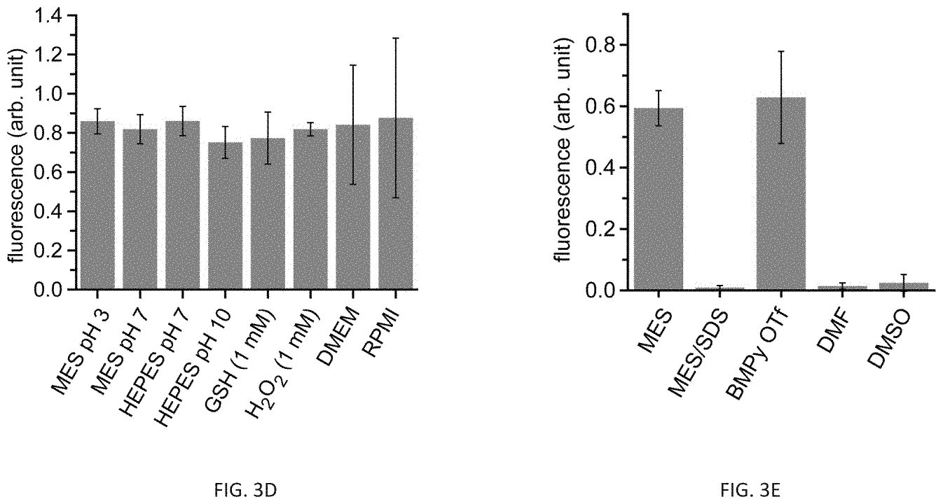

### Assessment of the Linkage Stability (a Procedure for FIG. 3D)

The solution of α-chymotrypsinogen A-biotin conjugate (2 μL, 0.1 mM in MES 50 mM) was mixed with buffer or medium containing 0.1% SDS (2 μL). The solution was incubated in a humidified chamber at 10° C. overnight or rt for 2 h and was spotted onto nitrocellulose membrane (1 μL/spot). After Ponceau S stain of the membrane, the membrane was washed with TBST buffer twice, blocked with 5% BSA at rt for 20 min, incubated with streptavidin-Cy5 conjugate (1:2,000) at rt for 40 min, washed with TBST buffer three times, and imaged by IMAGEQUANT® 800 (Cytiva, Upsala, Sweden). The experiment was triplicated on different days, and the fluorescence intensity was quantified by ImageJ software.

### Example 7

### Assessment of Streptavidin Activity after the Modification Conditions in Various Media (a Procedure for FIG. 3E.)

Streptavidin (75 μM) was incubated with potassium bicarbonate aqueous solution (20 mM), azide S5(7.5 mM), and PPh₃ or O═PPh₃ (7.5 mM) in different media at 37° C. for 2 h. For the condition in MES buffer with 0.1% SDS, the solution was heated at 95° C. for 1 min before the addition of azide and phosphine reagents to fully denature the protein. MES buffer (5 μL), H₂SO₄ aqueous solution (5 μL, 5% v/v), cold acetone (0.6 mL) was added to each reaction, and the solution was kept at −80° C. overnight. The precipitate was collected by centrifugation (15,000 rcf, 15 min, 4° C.), and the pellet was further washed by two additional cycles of acetone addition and centrifugation. The pellet was air-dried on bench at rt for 15 min after removing the final acetone solution. The dried pellet was reconstituted in ammonium carbonate buffer (5 μL, 5 mM).

For the binding assay, 2 μL of the reconstituted solution was mixed with 2 μL of 7:3 TBST buffer/glycerol solution containing 1.5 mM biotin-fluorescein (Sigma-Aldrich, 53608). The mixture was incubated at rt for 10 min and spotted onto PVDF membrane (0.5 μL/spot). The membrane was washed with methanol (2×30 sec) and imaged with IMAGEQUANT® 800 (Cytiva, Upsala, Sweden). After the fluorescence imaging, the membrane was subjected to Ponceau S stain.

For the total stain of the solubilized samples, 1 μL of the reconstituted solution was mixed with 1 μL of 7:3 TBST buffer/glycerol solution containing 8 M urea. The mixture was heated at 95° C. for 1 min and spotted onto PVDF membrane (0.5 μL/spot), and then the membrane was subjected to Ponceau S stain.

### Example 8

### Antibody and Antibody Fragment Modification and Purification

The modification reaction was performed in a 50-80-μL scale, following the procedure described in Typical peptide protein modification procedure in ionic liquid (Example 1). After the reaction at 37° C. for 2 h, cold PBS solution (1200 μL, 1× or 10×) was added, and any insoluble materials were separated by centrifugation (15,000 rcf, 15 min, 4° C.). The supernatant was diluted to 5 mL with cold PBS buffer, and concentrated to <0.5 mL by centrifugal filter (10k MWCO, 8,000 rcf, 30 min, 4° C.). The diluting- and concentrating-processes were repeated twice (3 times total centrifugation) to obtain antibody solution in PBS. The solution was used for the subsequent experiments without further purification, and aliquoted and stored at −80° C.

### Example 9

### Deglycosylation with PNGase F for MS Analysis

Modified antibody samples, after the cleanup processes either by acetone precipitation or centrifugal filter described above, were incubated with PNGase F (1 u/μL final concentration from 10 u/μL stock solution, Promega V4831, Promega Corporation, Madison, Wis., United States of America) in ammonium bicarbonate buffer (5 mM, pH 8) containing SDS (0.1%) at 37° C. for 2 h (typical final volume: 30 μL). TCEP solution (2 mM final concentration from 100 mM) in ammonium bicarbonate buffer (500 mM, pH 8) was added, and the mixture was incubated at rt for 20 min. Cold acetone (600 μL, −20° C.) was added in one portion, and the mixture was mixed by upside-down shaking and sit at −80° C. for 3 h. The precipitates were collected by centrifugation (15,000 rcf, 15 min, 4° C.), and the pellet was air-dried on bench at rt for 15 min after removing acetone. The dried pellet was reconstituted in ammonium bicarbonate buffer (5 mM, pH 8), and analyzed by MALDI-MS.

### Example 10

### Immunofluorescence Studies

SK-BR-3 was cultured and fixed in a 24-well culture plate with coverslips, according to the cell culture procedure described in the Materials and Reagents section above. The cells were blocked in a 4:1 mixture of 1×PBS/Blocking One (Nacalai Tesque Inc., Kyoto, Japan, 03953-95) solution at rt for 15 min. The blocking solution was removed, and the cells were incubated with primary antibody in 19:1 mixture of 1×PBS/Blocking One at rt for 1 h. Primary antibodies used are as follows: mouse anti-ErbB2 antibody (Invitrogen Technologies, Waltham, Mass., United States of America, e2-4001, 1:50 dilution); Herceptin (Carbosynth Ltd., Staad, Switzerland, FT65040, 1:1000 dilution, ˜0.2 μM final concn). Herceptin-SN-38 conjugate prepared by the tetrazene forming reaction (˜0.2 μM final concn), and Herceptin-biotin conjugate prepared by the tetrazene forming reaction (˜0.2 μM final concn). After the primary antibody treatment, the cells were washed with PBS solution three times and incubated with secondary antibody in 19:1 mixture of 1×PBS/Blocking One containing DAPI (0.01 mg/mL) at rt for 0.5 h. Secondary antibodies used are as follows: anti-mouse antibody Fab fragment-Cy5 conjugate (Jackson Immuno Research, West Grove, Pa., United States of America, 115-175-146, 1:100 dilution, ˜0.3 μM), anti-mouse antibody Fab fragment-Cy3 conjugate prepared by the urea forming reaction (1:30 dilution, ˜0.3 μM), anti-human antibody Fab fragment-Cy5 conjugate (Jackson Immuno Research, West Grove, Pa., United States of America, 709-175-149, 1:100 dilution), or streptavidin-Cy5 conjugate (Jackson ImmunoResearch, West Grove, Pa., United States of America, 016-170-084, 1.50 dilution). After the secondary antibody incubation, cells were washed with PBS solution three times. The coverslip with stained cells were mounted onto a microscope slide with a liquid mountant sold under the tradename PROLONG™ Gold Antifade Mountant (Thermo, P10144) and COVERGRIP™ Coverslip Sealant (Biotium, Fremont, Calif., United States of America, 23005), and the cells were imaged using confocal microscope.

### Example 11

### Preparative Synthesis of Small Molecules

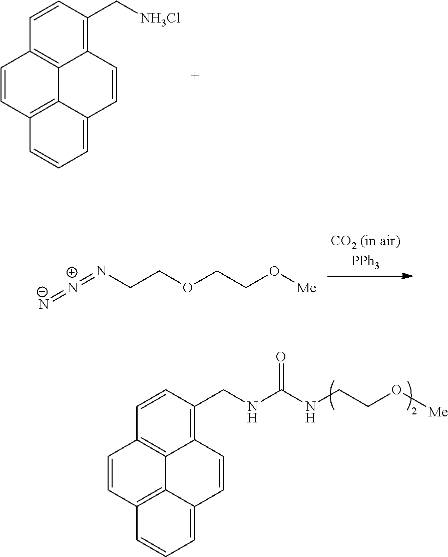

Synthesis of urea S2: Azide 2 (83.4 mg, 0.574 mmol) was added to a 4-mL vial equipped with a magnetic stir bar. BMPy OTf (0.1 mL), DMF (1.9 mL), DMSO (0.2 mL), and K₂CO₃ aq solution (0.045 mL, 5M, 0.225 mmol) were added. To the mixture, powder of (aminomethyl)pyrene hydrochloride S1 (17.4 mg, 0.065 mmol) was added. The mixture was heated at 50° C., and then PPh₃ (299.2 mg, 1.14 mmol) solution in DMF (0.9 mL) was added dropwise. After the reaction mixture was heated at 50° C. overnight, the formation of the product was confirmed by thin layer chromatography (R_{f}=0.5 with 98:1:1 dichloromethane/methanol/trimethylamine on basic aluminum oxide 60, MilliporeSigma #1057130001, Burlington, Mass., United States of America), and ether (4 mL) was added to the reaction mixture. The resulting suspension was filtered, and the solid was purified by thin layer chromatography (basic aluminum oxide 60, MilliporeSigma #1057130001, Burlington, Mass., United States of America) with 98:1:1 dichloromethane/methano/trimethylamine as eluents. The product was recovered from the alumina by addition of 4:1 acetonitrile/toluene mixture (40 mL) and sonication, and then the suspension was passed through diatomaceous earth (sold under the tradename CELITE®, Imerys Minerals California. Inc., San Jose, Calif., United States of America). After concentrating the filtrate under vacuum, the chromatography and CELITE® process was repeated, and removal of the volatiles under vacuum afforded the urea compound as off-white solids (3.7 mg, 15%). ¹H NMR (700 MHz, CD₃CN/DMSO-d₆ 95:5): δ 8.42 (d, J=9.2 Hz, 1H), 8.28 (t, J=7.4 Hz, 2H) 8.23 (t, J=7.4 Hz, 2H), 8.13 (s, 2H), 8.08 (t, J=7.7 Hz, 1H), 8.04 (d, J=7.8 Hz, 1H), 6.15 (br, 1H), 5.57 (br, 1H), 5.01 (d, J=5.8 Hz, 2H), 3.54 (m, 2H), 3.48 (t, J=5.3 Hz, 2H), 3.44 (m, 2H), 3.30 (q, J=5.5 Hz, 2H), 3.26 (s, 3H). ¹⁵N {¹H} NMR (60 MHz, CD₃CN/DMSO-d₆ 95:5, ¹⁵N-enriched sample): δ 302.6, 82.4. ESI-MS: calcd for C₂₂H₂₅N₄O₂ [M+H]⁺ 377.2, found 377.2.

Larger-scale synthesis of urea S2: Azide 2 (164.4 mg, 1.133 mmol) was added to a 20-mL vial equipped with a magnetic stir bar. BMPy OTf (0.22 mL), DMF (4 mL), DMSO (0.4 mL), and K₂CO₃ aq solution (0.09 mL, 5M, 0.45 mmol) were added. To the mixture, powder of (aminomethyl)pyrene hydrochloride S1 (32.5 mg, 0.122 mmol) was added. To the mixture, PPh; (698.6 mg, 2.663 mmol) solution in DMF (0.9 mL) was added in one portion, and the reaction vial was sealed with cap and electrical tape immediately. After the reaction mixture was heated at 50° C. overnight, the formation of the product was confirmed by thin layer chromatography (R_{f}=0.3 with 99:1 dichloromethane/methanol on basic aluminum oxide 60, MilliporeSigma #1057130001, Burlington, Mass., United States of America). Insoluble white solid was separate by filtration using MeCN (15 mL), and water (0.5 mL) was added to quench the reaction. After the removal of DMF and MeCN in vacuo, water (5 mL) was added, and the product was extracted with ether (5×10 mL). All the organic layers were combined and dried under vacuum. The resulting solid was purified by flash column chromatography (basic aluminum oxide 60, particle size 0.063-0.200 mm, 70-230 mesh ASTM, MilliporeSigma #1010671000: Burlington, Mass., United States of America) with 1:1 dichloromethane/hexane and then 80:20 dichloromethane/methanol as eluents, followed by preparative thin layer chromatography (basic aluminum oxide 60, MilliporeSigma #1057130001) with 99:1 dichloromethane/methanol as eluents. The product was recovered from the alumina plate by addition of methanol (3 mL) followed by sonication. And then 4:1 acetonitrile/toluene mixture (40 mL) was added, followed by sonication. The suspension was passed through CELITE® to remove alumina, and removal of the volatiles under vacuum afforded the urea compound as off-white solids (8.1 mg, 18%).

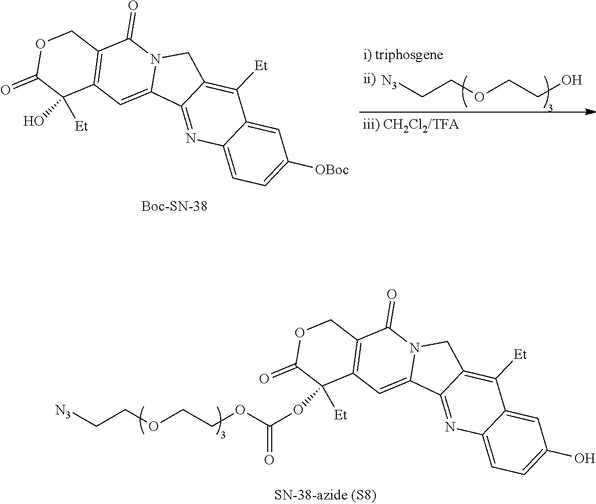

Synthesis of SN-38-azide (S8): Boc-SN-38 was prepared according to a previous literature report.⁵⁸ The carbonate formation and TFA deprotection procedures were adapted from a previous report.⁶⁰ Boc-SN-38 (32 mg, 0.065 mmol), DMAP (44.3 mg, 0.363 mmol), and triphosgene (9.4 mg, 0.032 mmol) were placed in a 2-mL vial. The vial was sealed, and N₂ gas was introduced. To the vial, dry CH₂Cl₂ (0.2 mL) was added in one portion. After the reaction mixture was stirred at rt for 5 min, 11-Azido-3,6,9-trioxaundecanol (38.3 mg, 0.175 mmol) was added dropwise, and the mixture was stirred at rt for 45 min. A 1:1 mixture of water/pyridine (0.1 mL) was added to quench the reaction, and the mixture was stirred at rt for 15 min. All the volatiles were removed by a gentle flow of nitrogen gas. The resulting oil was purified by silica gel column chromatography (CH₂Cl₂ with increase of methanol concentration from 0, 2, and 5% v/v) to afford Boc-protected SN-38-azide intermediate (21.1 mg). The intermediate was dissolved in CH₂Cl₂ (0.2 mL), and the solution was cooled in ice water bath for 2 min. Neat trifluoroacetic acid (0.2 mL) was added dropwise. After the solution was stirred in ice bath for 30 min, all the volatiles were removed by a gentle flow of nitrogen gas, and the resulting oil was purified by silica gel column chromatography (CH₂Cl₂ with increase of methanol concentration from 0, 2, and 5% v/v) to afford SN-38-azide as a yellow solid (9.2 mg, 22% over 3 steps). ¹H NMR (700 MHz, CDCl₃): δ 8.03 (d, J=9.1 Hz, 1H), 7.36 (dd, J=9.1, 2.6 Hz, 1H), 7.28 (d, J=2.5 Hz, 1H), 5.63 (d, J=16.8 Hz, 1H), 5.32 (d, J=16.8 Hz, 1H), 5.05 (d, J=2.5 Hz, 2H), 4.23 (m, 1H), 4.15 (m, 1H), 3.61 (m, 13H), 3.31 (t, J=5.2 Hz, 2H), 2.98 (q, J=7.7 Hz, 2H), 2.14 (m, 3H), 1.27 (t, J=7.7 Hz, 3H), 0.93 (t, J=7.5 Hz, 3H). ¹³C {¹H} NMR (176 MHz, CDCl₃): δ 167.5, 157.5, 155.6, 154.0, 149.4, 147.3, 146.0, 145.0, 143.8, 132.2, 128.5, 127.2, 122.5, 119.6, 105.6, 95.7, 78.1, 70.9, 70.8, 70.1, 68.8, 68.2, 67.2, 53.6, 50.8, 49.5, 32.0, 23.3, 13.8, 7.8. ESI-MS: calcd for C₃₁H₃₅N₅O₁₀ [M+H]⁺ 638.2, found 638.3.

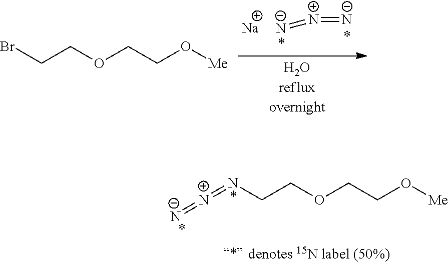

Synthesis of ¹⁵N-enriched azide (2-¹⁵N): The synthetic procedure was adopted from a previous report⁵⁷ but using ¹⁵N-enriched sodium azide (Millipore-Sigma #609374). To a 4-mL vial, 1-bromo-2-(2-methoxyethoxy)ethane (155.2 mg, 0.847 mmol) was added. Water (2.2 mL) and ⁵N-enriched sodium azide (138.5 mg, 2.10 mmol) were added, and the solution was refluxed overnight. After the solution was cooled to rt, brine (1.5 mL) and CH₂Cl₂ (2 mL) was added. The organic layer was separated, and the aqueous layer was further extracted with CH₂Cl₂ (4×2 mL). The combined organic layer was dried over MgSO₄, filtered, and dried in vacuo to afford ¹⁵N-enriched azide 2-¹⁵N as colorless oil (49.3 mg, 40%). ¹H NMR (6(0) MHz, CDCl₃): δ 3.69 (m, 4H), 3.59 (m, 2H), 3.43 (t, J=5.2 Hz, 2H), 3.42 (s, 3H). ¹⁵N {¹H} NMR (60 MHz, CDCl₃, ¹⁵N-enriched sample): δ 211.5, 68.8.

### Example 12

### Discussion of Examples 1-11

During the pursuit of novel protein labeling methods in ionic liquid, it was discovered that triphenylphosphine can induce a coupling reaction of amine groups of proteins with alkylazides. See FIG. 1A. Pyrrolidinium-based ionic liquid (1-butyl-1-methylpyrrolidinium trifluoromethanesulfonate, BMPy OTf) was chosen for initial study (see FIG. 1B), as this aprotic ionic liquid possesses good chemical stability²⁸ and has been successfully used for stabilizing proteins.²⁷ Incubation of amine-containing molecules with azide and phosphine reagents (i.e., Staudinger reaction reagents²⁹) in BMPy OTf induced the formal addition reaction of the entire carboxylate azide (1) onto amine groups under mild conditions (50° C. for 2 h) without release of dinitrogen. See FIG. 1C. In contrast, an appreciable level of the azide adduct was not observed in biologically benign media such as buffer and glycerol, indicative of the necessity of the aprotic solvent for the chemical transformation. See FIG. 1E.

A series of NMR spectroscopic data suggest that a urea group is formed upon the amine-azide coupling reaction. See FIG. 1D. To identify the product structure, alkylamine-substituted pyrene and a simple alkylazide (2) were chosen as model substrates. ¹H and 2D NMR spectra of the reaction product revealed the emergence of two broad peaks at 5.5 and 6.1 ppm (urea NHs) and their spatial relationship.^{30,31} Furthermore, using ¹⁵N-enriched azide on the terminal and alkyl-substituted nitrogen atoms (50% enrichment on each position³²), ¹⁵N-induced spin coupling was observed for one of the NH groups in the ¹H NMR spectrum, supporting the formation of an ¹⁵NH group. See FIG. 1D. Two peaks at 75 and 300 ppm were detected in the ¹⁵N NMR spectrum, corresponding to the signature peaks of nitrogen atoms of the NH groups. See FIG. 1D. Thus, all the spectroscopic data supports the formation of the urea group upon the described chemical reaction.

Even in the presence of a variety of NH-containing groups such as imidazole and guanidine, specific reactivity of the urea formation reaction was observed toward alkylamine groups of several peptide substrates with high reaction efficiency. See FIG. 2A. Consistent with this observation, an N-terminal amine-protected peptide without a lysine residue (LHRH) did not display meaningful reactivity. The modification sites were confirmed by tandem mass spectrometry (MS/MS) analysis. See FIG. 2B. The phosphine reagent proved necessary, as the corresponding phosphine oxide did not afford any detectable amount of product. See FIG. 2C. In addition, the compatibility of the reaction with various functional groups, such as carbamate and alcohol, in the azide reagent was also confirmed.

The urea formation reaction was amenable to chemical modification of protein substrates. See FIG. 3A. Mass spectrometry analysis of an HIV fusion inhibitor enfuvirtide³⁵ with two lysine residues and acetylated N-terminal amine group showed two modifications as predicted. Model protein substrates, lysozyme. α-chymotrypsinogen A, and streptavidin were subjected to the urea-forming reaction conditions, and, consistent with the number of lysine residues in each protein, multiple modifications were observed. The kinetics analysis of the protein modification showed that completion of the reaction can occur in less than 1 hour. See FIG. 3B. The applicability of the modification reaction to protein substrates was also confirmed by gel fluorescence analysis using a fluorophore azide in a phosphine-dependent manner. See FIG. 3C. Lysine residues were found to be the major modification sites of these reactions, as confirmed by pepsin digestion of the modified proteins and subsequent tandem mass spectrometry analysis.

The urea linkage is stable toward a multitude of biologically relevant species. To assess the stability of the modification linkage, biotinylated α-chymotrypsinogen A was produced using biotin-azide on a preparative scale. Even after exposure of the biotinylated protein to various conditions, the presence of more than 80% of biotin was observed by anti-biotin western blot, showing the retention of the modifications on the proteins (see FIG. 3D), which is consistent with the known structure- and substituent-dependent stability of urea groups. Retention of the modification was also confirmed on samples exposed to typical biochemical analysis conditions, such as exposure to a surfactant, heating (95° C.), and exposure to a reducing agent.

Protein activity was well retained even after treatment of the protein with the ionic liquid. The binding capability of streptavidin to biotin was chosen as a model system. The protein was exposed to azide and phosphine reagents in different types of media, and the binding capability of streptavidin to biotin-fluorophore was measured after buffer exchange. While meaningful fluorescence signals derived from the binding were observed for the buffer and ionic liquid-treated conditions, samples treated with high concentration of organic solvent (>90% DMF and DMSO) as well as denaturant (SDS)/heat provided almost no fluorescence signal (see FIG. 3E),³⁸ demonstrating the compatibility of the ionic liquid and the urea-forming reaction with this protein substrate.

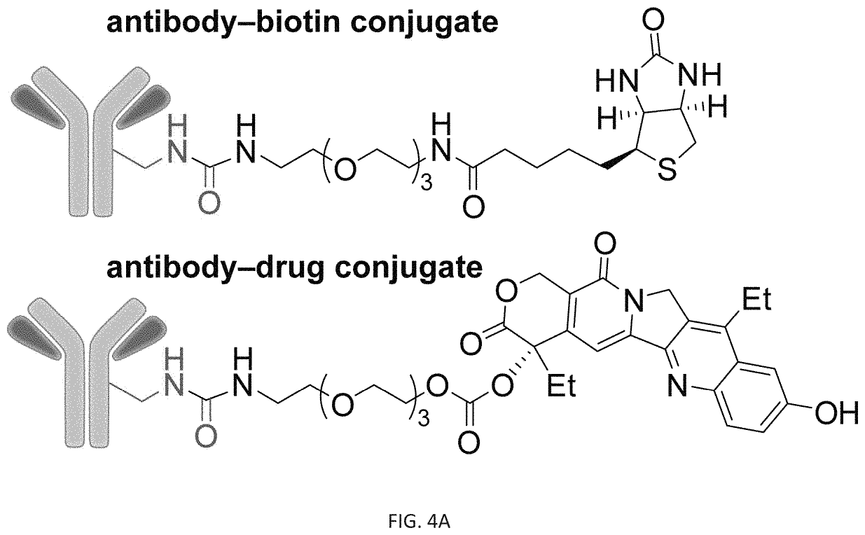

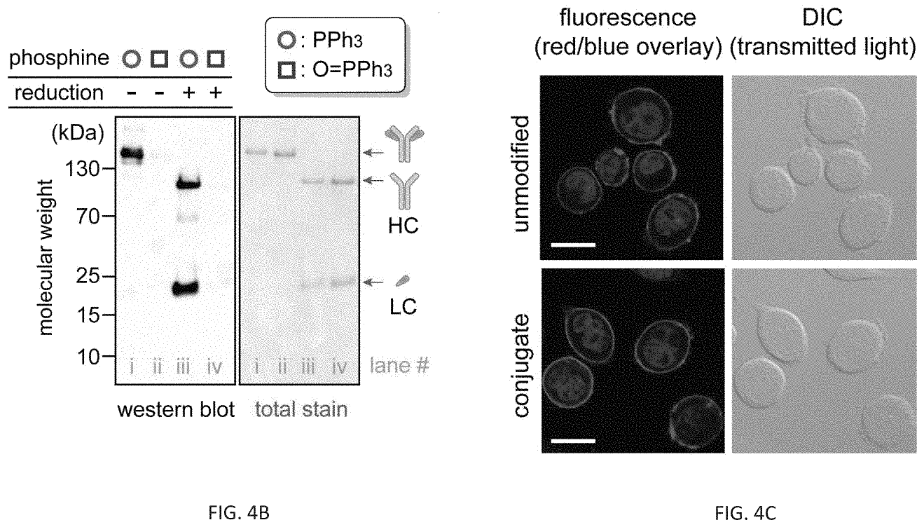

With the promising results of simple protein substrates, attention was turned to the modification of antibodies with a large molecular weight and more complicated structural motif. Initial investigations with polyclonal anti-mouse antibody Fab fragment with cyanine dye azide (Cy3-azide, 3″) resulted in successful attachment of the fluorophore onto the protein and its application to immunofluorescence experiments. Encouraged by the Fab fragment results, the urea-forming reaction was applied to a therapeutically relevant, full-length antibody, trastuzumab (sold under the tradename HERCEPTIN® (Genentech, Inc., South San Francisco, Calif., United States of America) used for breast cancer,³⁹ and its antibody-drug conjugates (ADCs. Trastzumab-emtansine) is an FDA-approved ADC.⁴⁰ Modification of HERCEPTIN® with biotin-azide demonstrated the installation of the affinity handle. See FIGS. 4A and 4B. Although phosphine compounds are typical agents for the fragmentation of antibodies through reduction of their disulfide bonds, the azide- and triphenylphosphine-based modification conditions did not show an appreciable level of fragmentation (see FIG. 4B), further supporting the compatibility of the urea-forming reaction with proteins.

The preparation of the antibody-drug conjugates (ADCs) by the urea modification reaction was also studied. SN-38 is a topoisomerase inhibitor used in the FDA-approved ADS sacituzumab govitecan (sold under the tradename TRODELVY® (Immunomedics, Inc., Morris Plains, N.J., United States of America).⁴¹ SN-38 with the alkylazide group and acid-labile carbonate linker was prepared by standard organic synthesis processes. The drug labeling process proceeded smoothly under the same reaction conditions as above, as confirmed by gel fluorescence and UV absorbance of SN-38. With the ADC in hand, the antigen recognition capability of the Herceptin-SN-38 conjugate was tested by confocal microscopy using SK-BR-3 cells, breast cancer cell lines. See FIG. 4C. Immunofluorescence signals of unconjugated HERCEPTIN® and the HERCEPTIN®-SN-38 conjugate produced virtually identical fluorescence signal intensity and patterns. Furthermore, staining with Herceptin-biotin conjugate and streptavidin-fluorophore conjugate produced the modification dependent fluorescence signal, corroborating the binding of the modified antibody to the antigen as well as the utility of the ionic-liquid reaction even for the modification of a large, intricate biomolecule.

The presently disclosed ionic liquid-based urea-forming amine-azide coupling reaction provided the efficient and selective bioconjugation strategy, and this study implies the presence of untapped opportunities for further development of click chemistry-like reaction in untraditional medium. Beyond bioconjugation fields, the present disclosure also highlights the breadth of the reactivity of azide groups behaving as electrophiles (i.e. umpolung reactivity);⁴² while phosphine-mediated azide reactions (i.e., phosphazide⁴²⁻⁴⁴ and iminophosphorane²⁹) are known to cause nucleophilic attack toward various electrophiles,⁴⁵⁻⁴⁷ covalent bond formation with a nucleophile (amine groups) has been unprecedented. Perhaps, ionic liquid-mediated reactivity enhancement plays a key role in the activation of the reaction intermediates,⁴⁸⁻⁵⁰ promoting the electrophilic nature of the phosphine-azide species to cause the ureabond forming reaction.^{51,52}

### Example 13

### General Methods, Materials and Instrumentation for Nucleic Acid Bioconjugation Studies

All the chemicals including DNAs were purchased from commercial vendor unless otherwise noted. A list of azides and DNAs used in this study is available in Table 5 and Table 6, below, respectively. All DNAs in Table 6 were purchased from Integrated DNA Technologies (Coralville, Iowa, United States of America) DNA ladder (Ultra low range, 10-300 bp) was purchased from Invitrogen (10597012; Waltham, Mass., United States of America). With the exception of azide 1e, azides in Table 5 were purchased as indicated from: Sigma-Aldrich (St. Louis, Mo. United States of America), Biosynth Carbosynth (Staad, Switzerland), Combi-Blocks (San Diego, Calif., United States of America), Synthonix, Inc. (Wake Forest, N.C., United States of America), AK Scientific (Union City, Calif., United States of America), Lumiprobe Corp. (Cockeysville, Md., United States of America), and Chem-Impex (Chem-Impex International, Wood Dale, Ill., United States of America). Structures of DNA modifiers are shown in Scheme 1, below. Benzodioxane-azide (1e) was synthesized according to the reported literature.¹⁰⁴

TABLE 5
Azide Compounds for Nucleic Acid Bioconjugation Studies.

| | Compound | | Supplier |
|---|---|---|---|
| Structure | # | Groups | (Catalog #) |
| 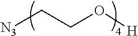 | 1a | Alcohol | Chem-Impex (NCI851841) |
| 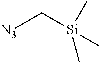 | 1b | Trimethyl- silyl (TMS) | Sigma- Aldrich (152854-5G) |
| 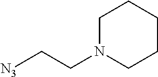 [SMILES: [N-]=[N+]=NCCN1CCCCC1] | 1c | Piperidine | Sigma- Aldrich (CDS015394) |
| 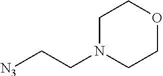 [SMILES: [N-]=[N+]=NCCN1CCOCC1] | 1d | Morpholine | Sigma- Aldrich (CDS005526) |
| 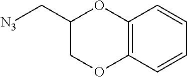 [SMILES: [N-]=[N+]=NCC1COc2ccccc2O1] | 1e | Benzodi- oxane | synthesized |
| 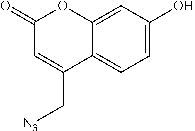 [SMILES: [N-]=[N+]=NCc1cc(=O)oc2cc(O)ccc12] | 1f | Coumarin | Synthonix (A73367) |
| 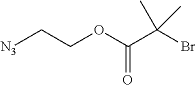 | 1g | Ester | Combi- Blocks (QH-5870) |
| 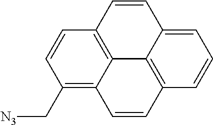 [SMILES: [N-]=[N+]=NCc1ccc2ccc3cccc4ccc1c2c34] | 1h | Pyrene | AK Scientific (AMTGC336) |
| 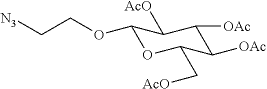 [SMILES: CC(=O)OCC1OC(OCCN=[N+]=[N-])C(OC(C)=O)C(OC(C)=O)C1OC(C)=O] | 1i | Acetyl- pyranose | Biosynth Carbosynth (MA34873) |
| 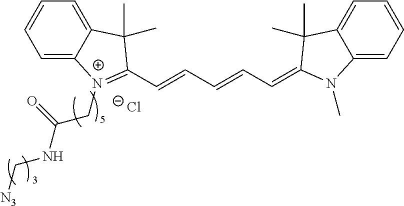 | 1j | Cy5 | Luiniprobe (41430) |
| 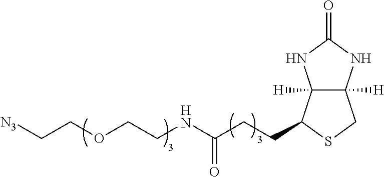 | S8 | Biotin | Sigma- Aldrich (762024) |

TABLE 6
Nucleic Acids.

| | | SEQ ID |
|---|---|---|
| Sequence | Name | NO: |
| 5′-/5AmMC12/TTT TT-3′ | TTTTT-5′-NH₂ | N/A |
| 5′-TTT TT/3AmMC6T/-3′ | 5′-TTTTT-3′-NH₂ | N/A |
| 5′-TTT/iAmMC6T/TT-3′ | 5′-TTT(NH₂)-TT-3′ | N/A |
| 5′-TTT TT-3′ | 5′-TTTTT-3′ | N/A |
| 5′-ATT TT-3′ | 5′-ATTTT-3′ | N/A |
| 5′-GTT TT-3′ | 5′-GTTTT-3′ | N/A |
| 5′-CTT TT-3′ | 5′-CTTTT-3′ | N/A |
| 5′-UTT TT-3′ | 5′-UTTTT-3′ | N/A |
| 5′-/5AmMC12/TGC GGT TGT AGT ACT CGT GGC | HSA aptamer-5′- | 9 |
| CG-3′ | NH₂ | |
| 5′-/5AmMC12/CCC TAG TTA GCC ATC TCC C-3′ | HIV-1-TAR | 10 |
| | aptamer-5′-NH₂ | |
| 5′-GGG AGA TGG CTA ACT AGG G/3Cy5Sp/-3′ | Complementary | 11 |
| | HIV-2-TAR- | |
| | aptamer-3′-Cy5 | |
| 5′-/5AmMC12/GCA GCG GTG TGG GGG CAG CGG | Her2-aptamer-5′- | 12 |
| TGT GGG GGC AGC GGT GTG GGG-3′ | NH₂ | |
| 5′-GCA GCG GTG TGG GGG CAG CGG TGT GGG | Her2-aptamer-3′- | 13 |
| GGC AGC GGT GTG GGG/3AmMO/-3′ | NH₂ | |
| 5′-GCA GCG GTG TGG GGG CAG CGG TGT GGG | Her2 aptamer | 14 |
| GGC AGG GGT GTG GGG-3′ | | |
| 5′-CCC CAC ACC GCT GCC CCC ACA CCG CTG | Complementary | 15 |
| CCC CCA CAC CCC TGC-3′ | HER2 aptamer | |
| 5′-CCCTAGTTAGCCATCTCCC-3′ | HIV-1-TAR aptamer | 16 |

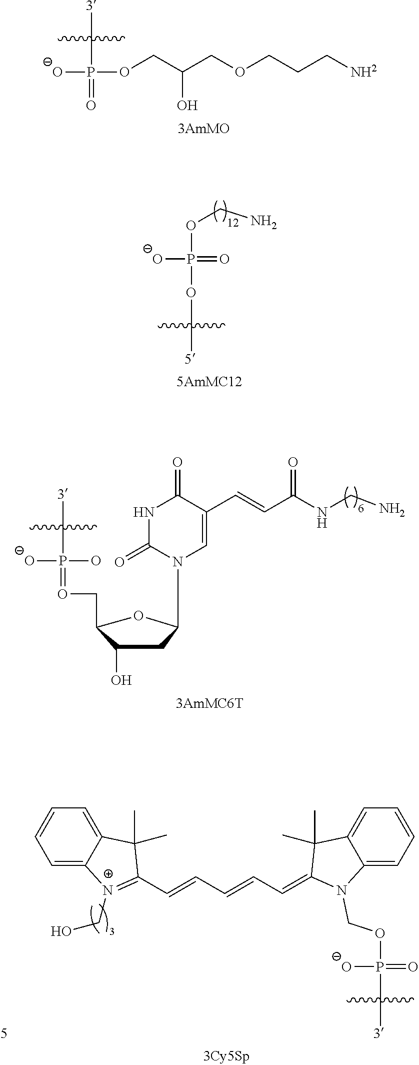

### Scheme 1. Structures of DNA Modifiers.

MALDI-MS was performed on a Bruker Daltonics Autoflex-TOF (Brucker, Billerica, Mass., United States of America). Matrices used were 20 mg/mL 3,4-diaminobenzophenone (DABP) in ammonium citrate (20 mg/mL solution in H₂O)/acetonitrile (1:1) or saturated solution of 3-hydroxypicolinic acid (3-HPA) in ammonium citrate (20 mg/mL solution in H₂O)/acetonitrile (1:1). For DABP, a 1:1 ratio of sample (0.5 or 1 μL) and matrix solution (0.5 or 1 μL) were mixed on a ground-steel MALDI plate (8280784, Bruker, Billerica, Mass., United States of America). For 3-HPA, a 2:1 ratio of matrix (1 μL) and sample (1 μL) were used; first, 3-HPA (1 μL) was added and dried, then sample (1 μL) was added and dried, and another 3-HPA (1 L) was added and dried on the same well. The DABP matrix solution was freshly prepared every time, and the 3-HPA matrix solution was prepared every 1 month and stored at −80° C.

LC-MS was performed on Thermo Vanquish LC system and LTQ-XL linear ion trap MS system with a C18 column (sold under the tradename HYPERSIL GOLD™ 25003-032130, particle size: 3 μm, diameter: 2.1 mm, length: 30 mm) (all from Thermo Fisher Scientific (Waltham, Mass., United States of America). The flow rate was 0.4 mL/min. Triethylammonium acetate buffer (5 mM, pH 7.2) was used as eluent with the gradient of acetonitrile (5-40% for 3.5 min, and then 90% for 1.5 min). The analysis of the reactions was performed by the UV detection of the unmodified DNA at 254 nm, compared with the internal standard (single stranded DNA, TAUCG (0.05 mM). The conversion of the reaction was calculated by the decrease of the peak area of starting material (unmodified DNA) using the internal standard.

Agarose gel electrophoresis was performed as described below. 4.0 g of Agarose (product number A20090, Research Products International, Mount Prospect, Ill., United States of America) was suspended in 100 mL of TAE Buffer (10×EMD Millipore 574797 (Millipore Sigma. Burlington, Mass., United States of America), 4% w/v final concentration) and 10 times diluted with water. The agarose was dissolved by heating in a microwave (1050 W) for 2 min. For the total staining purpose, 1.5 mL of SYBR® Gold nucleic acid gel stain (S11494, Invitrogen, Waltham, Mass. United States of America) was added while stirring. The hot solution was poured into the container of the Electrophoresis Unit (Walter EL-100, Walter Products, Inc., Tecumseh, Ontario, Canada) and cooled at rt for 20 min. In a 1.7-mL Eppendorf tube, 1 μL of the samples were mixed with 5 μL of TRACKIT™ Cyan/Yellow Loading Buffer (10482035, Invitrogen, Waltham, Mass., United States of America), and the mixed samples were loaded to the gel. 1 μL of Ultra Low Range DNA Ladder (10597012; Invitrogen, Waltham, Mass., United States of America) was mixed with 1 μL of TRACKIT™ Cyan/Yellow Loading Buffer (10482035; Invitrogen, Waltham, Mass., United States of America), and the mixed solution was loaded to the gel. The electrophoresis was run by using the power supply (BIO-RAD POWER PAC™ Basic Supply 1645050, Bio-Rad Laboratories, Hercules, Calif., United States of America) for 40-50 min at constant 150 V. The resulting gel was analyzed by Amersham IMAGEQUANT® 800 (Cytiva, Upsala, Sweden).

Gel fluorescence and southern blot imaging was conducted on Amersham IMAGEQUANT® 800 (Cytiva, Upsala, Sweden). Gel or blot fluorescence imaging were performed using 460-nm (Cy2), 535-nm (Cy3), and 635-nm (Cy5) light sources with corresponding emission bandpass filters at 525 nm (±20 nm), 605 nm (±40 nm), and 705 (±40 nm), respectively. Anti-biotin southern blot was performed with streptavidin-Cy5 conjugate (016-170-084, Jackson ImmunoResearch (West Grove, Pa., United States of America), 1:2,000 dilution for southern blot) after blocking with 5% BSA in TBST buffer. Quantification of the blot membrane images were performed by using ImageJ software, and signals were normalized to one of the strongest signals (set as 1.0) in the experiment. Weak, unanalyzable signals in the Image J software were set as 0.01, compared to the normalization sample (1.0).

Confocal microscopy: Fluorescence microscope imaging was performed on Zeiss scanning microscope 710 with a 40× water immersion C-Apochromat objective lens (numerical aperture 1.1)(Carl Zeiss A G, Oberkochen, Germany). Excitation at 405 nm (DAPI), 488 nm (Phalloidin-CF488), and 633 nm (Cy5) were used with filter settings 410-480 nm, 494-631 nm, and 638-759 nm, respectively. Image J software was used to generate images suitable for publication.

NMR was performed on Bruker AVANCE NEO 600 and 700 (Brucker, Billerica, Mass., United States of America).

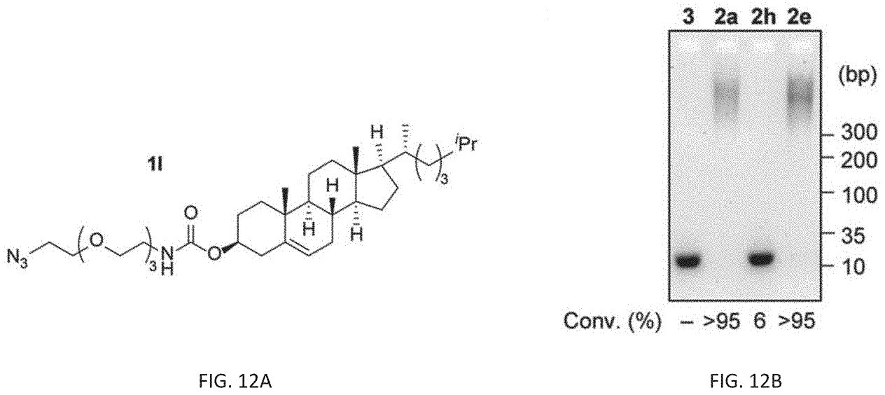

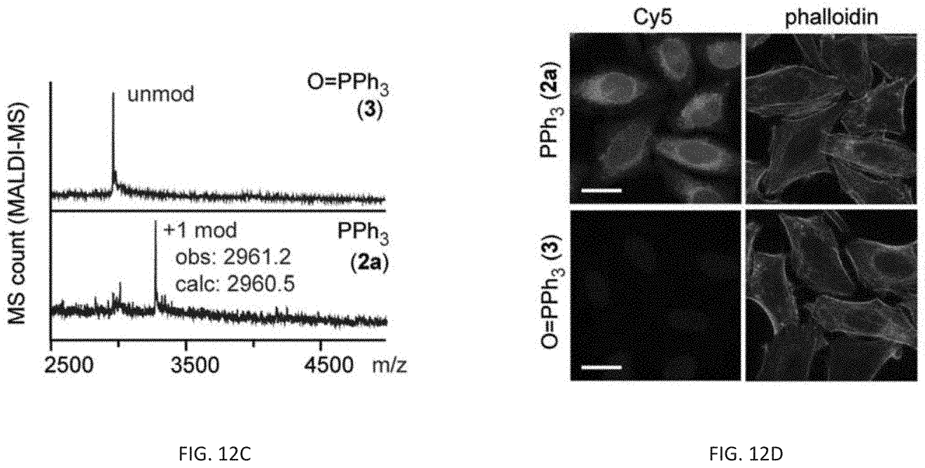

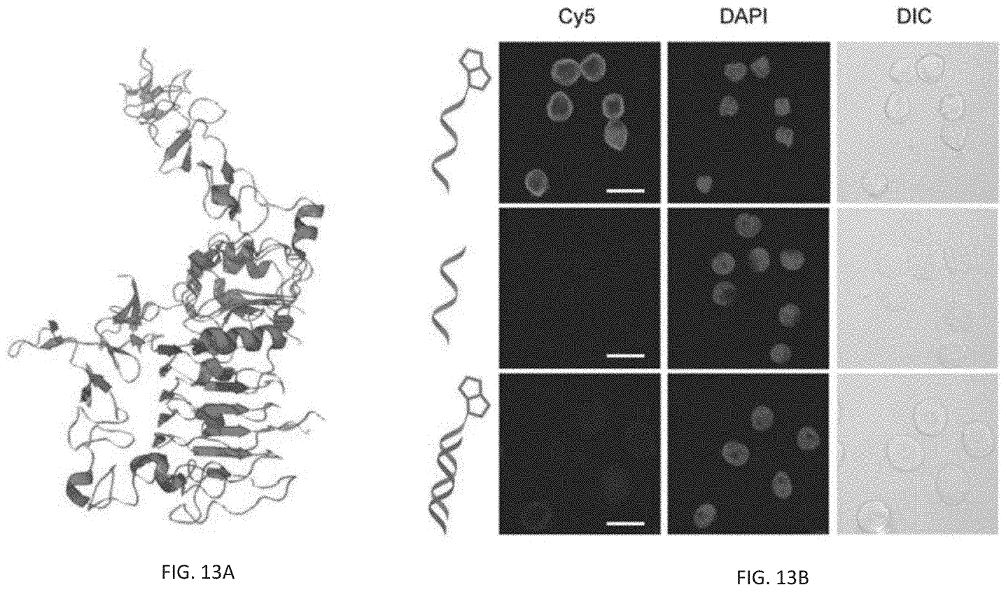

Cell culture: SK-BR-3 and HeLa cells were cultured in Dulbecco's Modified Eagle Medium (DMEM) with Glutamax and 10% fetal bovine serum (FBS) using 24-well cell culture plate (Corning 3524) coated with poly-L-lysine under 5% CO₂ at 37° C. Cells were fixed with 4% paraformaldehyde at 90% (SK-BR-3 cells) or 60% (HeLa cells) confluency, washed with PBS three times, and used for cell staining experiments. See FIGS. 12D and 13B.

### Example 14

### Typical DNA Modification Procedure in Ionic Liquids

To ionic liquids (typically 10-40 μL for analytical scale), potassium bicarbonate aqueous solution (20 mM final concentration from 2M stock solution in water), DNA aptamers or pentanucleotides (0.02-0.1 mM final concentration from 2-5 mM stock solution in water), alkyl azide (3-7.5 mM final concentration from 100-500-mM stock solution in DMSO), and PPh₃ or O═PPh₃ (3-20 mM final concentration from 150-500-mM stock solution in DMSO) were added. The final concentration of H₂O was kept lower than 6% v/v. The reaction mixture was incubated in a 50° C. incubator for 2 h and subjected to a Post-reaction cleanup process for analytical scale reaction before analysis.

### Example 15

### Post-Reaction Cleanup Process for Analytical Scale Reaction

To the reaction mixture (typically 10-40 μL) in a 1.7-mL Eppendorf tube, cold acetone (600-900 μL, −20° C.) was added in one portion. For the samples in EMIM OAc, the precipitation was performed with cold acetone/methanol (5:1 ratio, 600-900 μL, −20° C.) instead of cold acetone. After the addition of acetone or acetone/methanol, the mixture was mixed by upside-down shaking and sit at −80° C. for 1 h or overnight. The precipitates were collected by centrifugation (15,000 rcf, 15 min, 4° C.), and acetone or acetone/methanol was removed. The pellet was further washed by acetone or acetone/methanol (5:1) addition and centrifugation processes before the pellet was air-dried on the bench at rt for 15 min. The dried pellet was reconstituted in 10-30 μL of ammonium bicarbonate (NH₄HCO₃) aqueous solution (5 mM) and analyzed by suitable analytical methods.

### Example 16

### Southern Blotting (Dot Blot)

The reconstituted samples (0.5 μL) were spotted onto a positively charged nylon membrane (11209299001; Roche Holding AG, Basel, Switzerland). Mayer hemalum solution for the membrane stain was prepared by diluting the commercial solution (1.09249.0500 (Sigma-Aldrich, St. Louis, Mo., United States of America) with water (100 times dilution) in a 50-mL falcon tube, and the diluted solution can be stored at rt. The membrane with the DNA was stained with the diluted Mayer hemalum solution for 5 min and rinsed with TBST buffer twice. The stained membrane was imaged by IMAGEQUANT® 800 (Cytiva. Upsala, Sweden) to obtain the colorimetric image. Then the membrane was washed with TBST buffer for 5 min, blocked with 5% BSA in TBST buffer at rt for 20 min, incubated with streptavidin-Cy5 conjugate (1:2000) in the blocking buffer at rt for 40 min, washed with TBST buffer three times, and imaged by IMAGEQUANT® 800 (Cytiva, Upsala, Sweden). For the quantification purpose, the experiment was triplicated on different days, and the fluorescence intensity was quantified by ImageJ software.

### Example 17

### Pentanucleotide Modification with Azide 1a

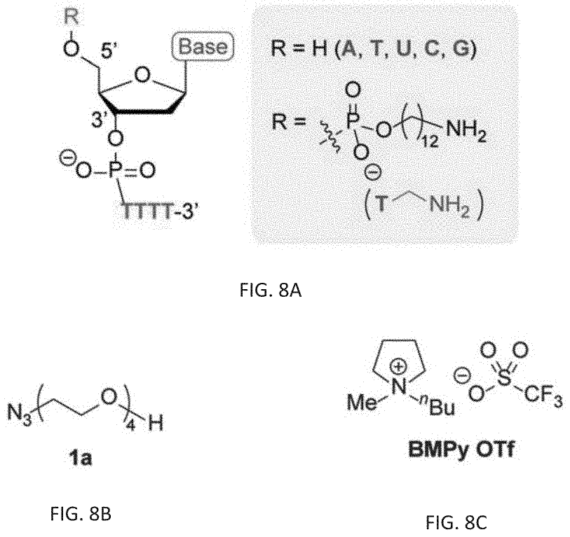

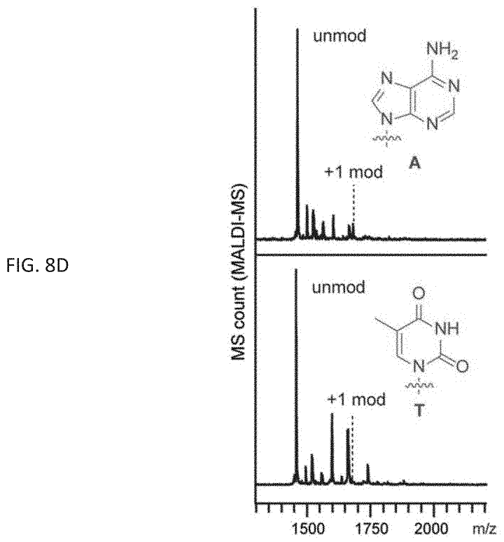

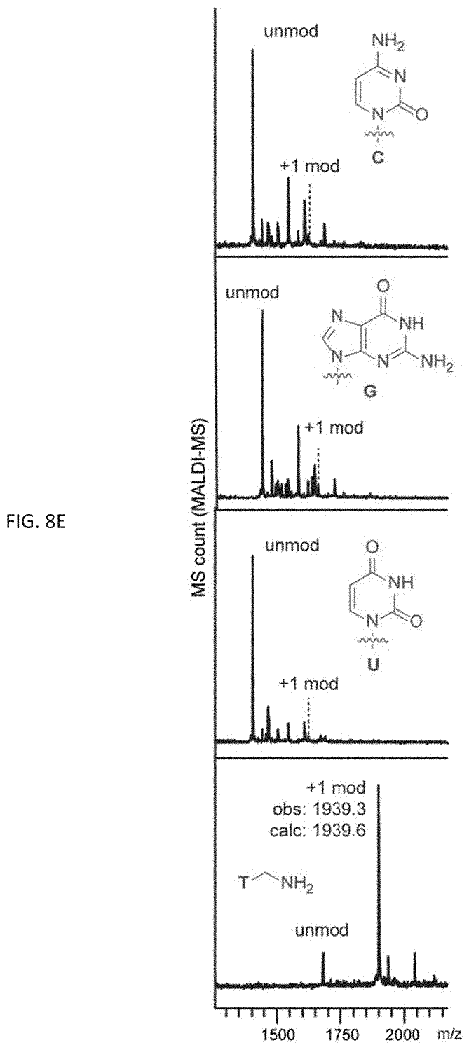

The urea-forming reaction on pentanucleotides (5′-XTTTT-3′, where X=adenosine, thymidine, cytosine, guanosine, deoxyuridine, and thymidine with alkylamine containing 12 carbon linker) were performed by Typical DNA modification procedure in ionic liquids (see Example 14) and Post-reaction cleanup process for analytical scale reaction (see Example 15) in 30 μL scale with following conditions. 5′-XTTT-3′ (0.2 mM final concentration from 5 mM stock solution in H₂O), KHCO₃ (20 mM final concentration from 2 M stock solution in H₂O), azide 1a (7.5 mM final concentration from 250 mM stock solution in DMSO), and PPh₃ (20 mM final concentration from 500 mM stock solution in DMSO) in BMPy OTf at 50° C. for 2 h. See FIGS. 8A-8D.

### Example 18

### Kinetics Studies

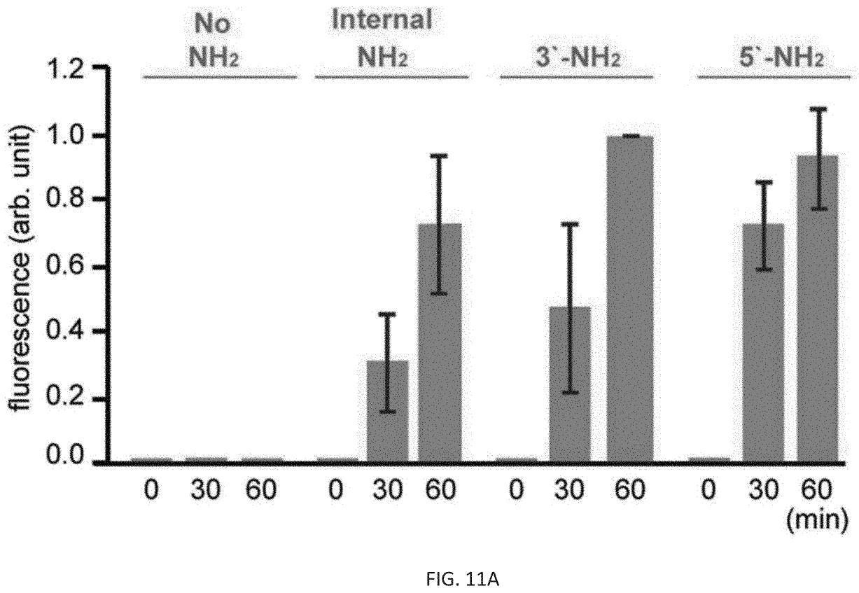

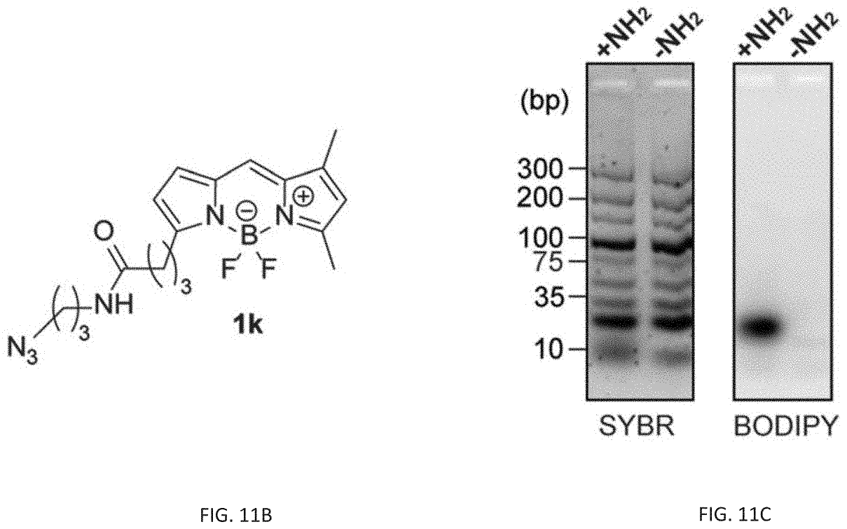

The urea-forming reaction on pentathymidine for the kinetics investigation were performed by Typical DNA modification procedure in ionic liquids (see Example 14) and Post-reaction cleanup process for analytical scale reaction (see Example 15) in 20 μL scale with following conditions. The resulting DNA solution was spotted in a nylon membrane. Once the spots get dried, the membrane was rinsed with MeOH once and visualized by fluorescence (BODIPY). After that, the membrane was stained by the diluted MHS (see Southern blotting in Example 16) for 5 min, rinsed with TBST buffer twice, and visualized by IMAGEQUANT® 800 (Cytiva, Upsala, Sweden). Standard deviations (error bars, n=3) and coefficient of determination (R-squared test) were calculated after three independent replicates. Reaction conditions: Pentathymidine (0.2 mM final concentration from 5 mM stock solution in H₂O), KHCO₃ (20 mM final concentration from 2 M stock solution in H₂O), BODIPY azide 1k (3 mM final concentration from 100 mM stock solution in DMSO), and PPh₃ (3 mM final concentration from 150 mM stock solution in DMSO) in BMPy OTf at 50° C. forgiven time (0, 30, 60 min). See FIG. 11A.

### Example 19

### Modification of Alkylamine-Tagged DNA in the Presence of DNA Ladder

To 10 μL of Ultra Low Range DNA Ladder (10597012, Invitrogen (Waltham, Mass., United States of America)), 1 μL of 3 M sodium acetate at pH 5.2 and 30 μL of ice-cold 100% ethanol were added. DNA samples were mixed and stored at −20° C. for 1 h to precipitate DNA. DNA pellet was collected by centrifugation (15,000 rcf, 15 min, 4° C.), and then supernatant was removed. And the pellet was washed with ice-cold 70% ethanol two times with the centrifugation processes. The pellet was air-dried on the bench at rt for 15 min after removing ethanol and reconstituted with 5 μL of distilled water. The concentrated DNA solution was analyzed by NANODROP® 2000 (Thermo Fisher Scientific, Waltham, Mass., United States of America).

The pentanucleotides were modified with BODIPY azide 1k in the presence of DNA Ladder by Typical DNA modification procedure in ionic liquids (see Example 14) and Post-reaction cleanup process for analytical scale reaction (see Example 15) in 20 μL scale with following conditions. T-TMT-5′-NH₂ (0.05 mM final concentration from 1 mM stock solution in H₂O), Ultra Low Range DNA Ladder (0.02 μg/μL final concentration from 0.9 μg/μL stock solution in H₂O), KHCO₃ (20 mM final concentration from 2 M stock solution in H₂O), BODIPY azide 1k (3.75 mM final concentration from 100 mM of stock solution), and PPh₃ (3 mM final concentration from 150 mM stock solution in DMSO) were added into BMPy OTf: DMSO: DMF (2:1:1 ratio). After the acetone precipitation process, the reconstituted solution was added 6×DNA Loading dye (1 μL) and run by 4% agarose gel premade with SYBR® Gold nucleic acid gel stain (1,5-2:10000) or plain gel for 40 min at constant 150 V. Total DNA samples was visualized by the fluorescence from SYBR® Gold nucleic acid gel stain (Cy3), whereas modified DNA samples were visualized by the fluorescence from BODIPY (Cy2). See FIG. 11C.

### Example 20

### Representative Procedure for Formation of DNA Duplexes

The single-stranded DNA (5-9 μL, 0.02-0.1 mM final concentration from 0.04-0.2 mM stock solution in H₂O) and its complementary sequence (5-9 μL, 0.02-0.1 mM final concentration from 0.04-0.2 mM stock solution in H₂O) was hybridized by heating at 60° C. for 10 min using Digital Dry Bath (product number 88870002; Thermo Fisher Scientific (Waltham, Mass., United States of America)).

### Example 21

### Cell Staining with Cholesterol Modified DNA

The cholesterol modified DNA was prepared by Typical DNA modification procedure in ionic liquids (see Example 14) and Post-reaction cleanup process for analytical scale reaction (See Example 15) in 30 μL scale with following conditions. HIV-1-TAR-5′-NH₂ aptamer (SEQ ID NO: 10, 0.1 mM final concentration from 5 mM stock solution in H₂O). KHCO₃ (20 mM final concentration from 2 M stock solution in H₂O), cholesterol azide 11(7.5 mM final concentration from 100 mM stock solution in DMSO/toluene (7:3)), and PPh₃ or O═PPh₃ (20 mM final concentration from 500 mM stock solution in DMSO) in EMIM OAc/DMF/DMSO (2:1:1 ratio) at 50° C. for 2 h. HeLa cells were cultured and fixed in a 24-well culture plate with coverslips, according to the procedure described in Cell culture (see Example 13). The cells were blocked in a 4:1 mixture of 1×PBS/Blocking One (03953-95, Nacalai Tesque (Kyoto, Japan)) solution at rt for 15 min. The blocking solution was removed, and the cells were incubated with dsDNA (final concentration ˜2 μM) and phalloidin-CF488 conjugate (1:40 dilution, final concentration 5 U/mL, Biotium (Fremont, Calif., United States of America), 00042) in 19:1 mixture of 1×PBS/Blocking One at rt for 30 min. For the preparation of dsDNA solution, the cholesterol modified (PPh₃) or unmodified (O═PPh₃) ssDNA was hybridized with Cy5 conjugated complementary ssDNA, according to the procedure described in Assembly of DNA duplexes (see Example 20). After the incubation, cells were washed with PBS solution three times. The coverslip with stained cells were mounted onto a microscope slide with PROLONG GOLD™ Anti-fade Mountant (P10144, Thermo Fisher Scientific, Waltham, Mass., United States of America) and CoverGrip Coverslip Sealant (23005, Biotium. Fremont, Calif., Untied States of America), and the cells were imaged using confocal microscope. See FIGS. 12A-12D.

### Example 22

### Cell Staining with Biotin Modified Her2 Aptamer DNA

The biotin modified DNA was prepared by Typical DNA modification procedure in ionic liquids (see Example 14) and Post-reaction cleanup process for analytical scale reaction (see Example 15) in 30 μL scale with following conditions. Her2 5′-NH₂ aptamer (SEQ ID NO: 12, 0.1 mM final concentration from 2 mM stock solution in H₂O). KHCO₃(20 mM final concentration from 2 M stock solution in H₂O), biotin azide S8 (7.5 mM final concentration from 250 mM stock solution in DMSO), and PPh₃ or O═PPh₃ (20 mM final concentration from 500 mM stock solution in DMSO) in EMIM OAc/DMF/DMSO (2:1:1 ratio) at 50° C. for 2 h. SK-BR-3 cells were cultured and fixed in a 24-well culture plate with coverslips, according to the procedure described in Cell culture (see Example 13). The cells were blocked in a 4:1 mixture of 1×PBS/Blocking One (03953-95, Nacalai Tesque, Kyoto, Japan) solution at rt for 15 min. The blocking solution was removed, and the cells were incubated with ssDNA (biotin-modified or unmodified) or ds DNA (biotin-modified) at the final concentration of ˜0.5 μM in 19:1 mixture of 1×PBS/Blocking One at rt for 1 h. For the preparation of dsDNA solution, the biotin modified ssDNA (PPh₃) was hybridized with complementary ssDNA, according to the procedure described in Assembly of DNA duplexes (see Example 20). For the sake of consistency across the samples, ssDNAs were also heated at 60° C. for 10 min by the same method before used in the cell staining. After the incubation with the DNA solutions, the cells were washed with PBS solution three times and incubated with Streptavidin Cy5 (016-170-084 (Jackson ImmunoResearch, West Grove, Pa., United States of America), 1:100 dilution) in 19:1 mixture of 1×PBS/Blocking One containing DAPI (0.1 μg/mL) at rt for 30 min. After the incubation, cells were washed with PBS solution three times. The coverslip with stained cells were mounted onto a microscope slide with PROLONG™ Gold Antifade Mountant (P101441, Thermo Fisher Scientific, Waltham, Mass., United States of America) and COVERGRIP™ Coverslip Sealant (23005, Biotium, Fremont, Calif., United States of America), and the cells were imaged using confocal microscope. See FIGS. 13A and 13B.

### Example 23

### Mayer Staining Procedure

A pentanucleotides, 5′-TAUCG-3′, sample was diluted as 0.05, 0.1, 0.2, 0.4, 0.8 mM, spotted in a nylon membrane, stained with the diluted MHS (see Example 16) for 5 min, dried overnight, and imaged by IMAGEQUANT® 800 (Cytiva, Upsala, Sweden). The experiment was repeated three times to obtain the standard deviation (error bars, n=3) for the images.

### Example 24

### Gel Shift Assay for the Her2 Aptamer Modification

The biotin modified DNA for the gel shift assay was prepared by in 24 μL scale with following conditions. Her2 aptamer-5′-NH₂ (SEQ ID NO. 12, 5 μM final concentration from 0.2 mM stock solution in 5 mM NH₄HCO₃ aq.) modified (PPh₃) or unmodified (OPPh₃) with biotin azide S8 was incubated with or without streptavidin (25 μM final concentration from 1 mM stock solution in 50 mM MES buffer) at rt for 20 min. 5 μL of the resulting mixture was analyzed by the agarose gel electrophoresis and the DNA was visualized by SYBR® Gold total staining.

### Example 25

### Discussion of Examples 13-24

Reactivity survey of the phosphine-mediated chemistry: An initial survey of the reactivities of the adenine (A), thymine (T), cytosine (C), guanine (G), and uracil (U) nucleotides in a simple DNA substrate revealed their inertness toward the phosphine-azide coupling reaction. See FIGS. 8A-8D. In order to understand the applicability of the phosphine-azide coupling reaction, nonfunctional azide reagent 1a was first applied to a tetrathymidine containing an additional nucleotide at the 5′ terminus. See FIGS. 8A-8C. Notwithstanding the presence of different types of NH₂ groups in native DNAs, virtually no urea-modified DNAs were observed in mass spectrometric analysis after the reaction and subsequent buffer exchange processes. See FIGS. 8D and 8E. On the other hand, introduction of alkylamine at the 5′-terminus of the backbone led to a significant increase in the signal from the peak corresponding to the formation of the urea group. See FIG. 8E, bottom. Those results suggest that the ionic liquid-based amine-azide coupling has a high specificity for alkylamines over other endogenous arylamine groups.

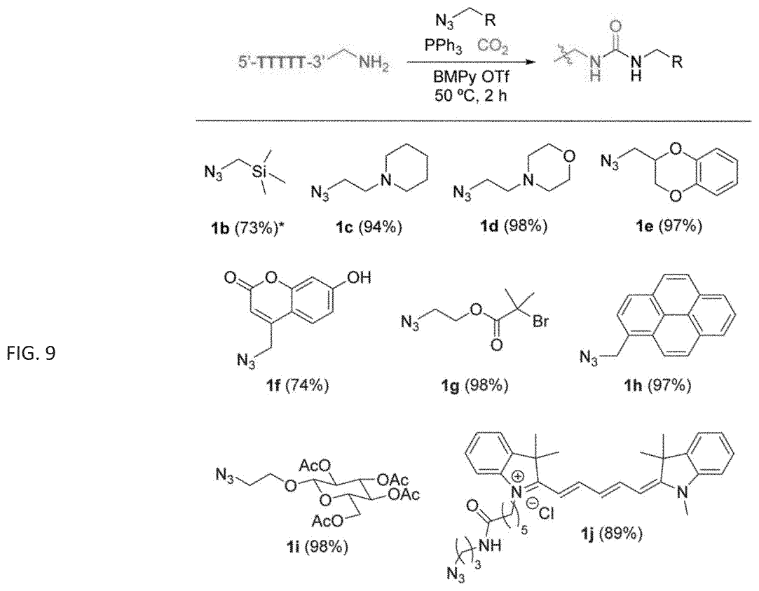

The high functional group tolerance of the modification process allowed incorporation of a variety of alkylazide reagents to the alkylamine-tagged DNA. See FIG. 9. The trimethylsilyl (TMS) group has been used increasingly as a chemical reporter in structural biology research because of its characteristic chemical shift in nuclear magnetic resonance (NMR) spectra.^{87,88} Despite the relatively large steric bulk of the trimethylsilyl group, the TMS-methylazide (1b) could be used to introduce the TMS group to the DNA. Tertiary amine-containing reagents such as 1c or 1d did not decrease the efficiency of the modification and could be useful for introduction of an additional positive charge to the biomolecule. Incorporation of the morpholine group (1d) is noteworthy as the resulting tag could be useful in endoplasmic reticulum-targeting applications.⁸⁹ Aromatic rings and a fluorophore scaffold are also compatible with the modification, enabling preparation of a color-palette of DNA-fluorophore conjugates. Interestingly, despite the versatile reactivities of azides, phosphines, and their derivatives (e.g. phosphazide and iminophosphorane), a single product was observed with an alkyl bromide substituted azide (1g) without loss of the bromide group. As the incorporation of the dimethylalkyl bromide is common in radical polymerization processes,⁹⁰⁻⁹² the successful attachment of the group assists further development of DNA-polymer conjugates. Thus, the phosphine azide reaction is not affected by a series of functional groups such as silyl, tertiary amino, ether, ester, aryl alcohol, tertiary alkyl bromide and alkene groups.

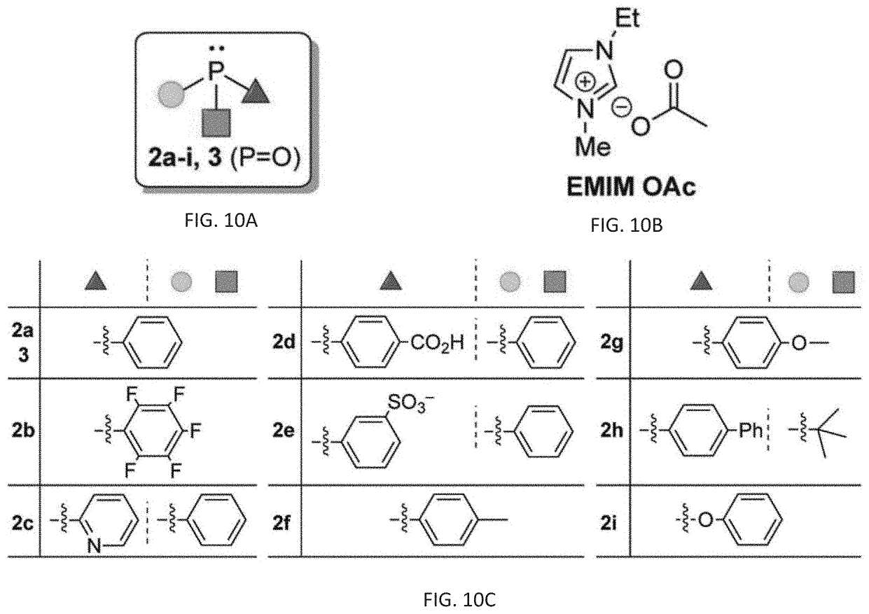

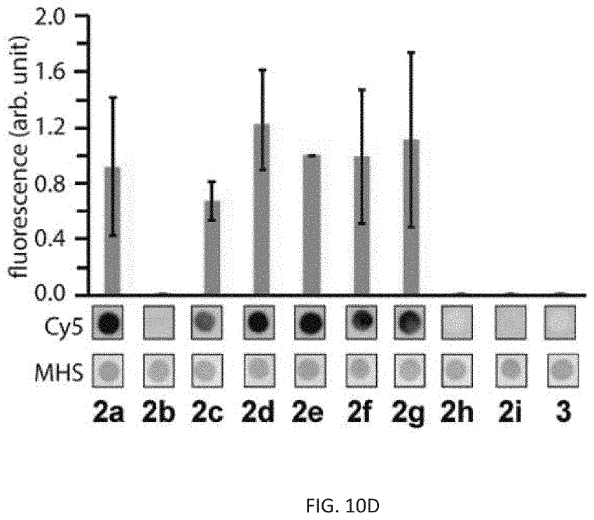

In an effort to understand the compatibility of the functional groups and enhance the reaction efficiency, different types of phosphine and phosphite reagents were used. See FIGS. 10A-10D. A DNA aptamer for human serum albumin was chosen as a model substrate for this study of phoshines.⁹³ Reasonably air-stable phosphines including triaryl-phosphines with a range of substituents (2a-2g), alkylphosphine (JohnPhos, 2h), and arylphosphite (2i) were used and triphenylphosphine oxide (3) was employed as a negative control.⁹⁴ See FIGS. 10A and 10C. The degree of the modification reaction was assessed with biotin-azide which provides an analysis handle for anti-biotin southern blotting with a streptavidin-fluorophore conjugate. With the sole exception of tris(pentafluorophenyl)phosphines (2b), all the triarylphosphines (2a, 2c-2g) displayed a fluorescence signal at a similar level, indicating the minor effect of the subtle changes of the electronic properties of the aryl substituents. See FIG. 10D. On the other hand, substantially weaker fluorescence signals were observed from reactions involving bisalkyl-monoarylphosphine (2h) or triphenylphosphite (2i). In order to visualize the total DNA amount on a blot membrane, Mayer's hemalum solution, which relies on coordination of aluminum with the phosphate backbone, was employed.⁹⁵ In contrast to the southern blot showing a varied fluorescence signal dependent on the biotin attachment on DNA, Mayer's hemalum stain showed similar intensity across all the conditions, confirming the validity of the experimental design. The triarylphosphine-dependent modification was also confirmed by mass spectrometry. Different types of ionic liquid could be utilized for this chemical transformation. See FIG. 10B. Furthermore, double-stranded DNAs with the alkylamine tag exhibited comparable reactivity to the single-stranded DNA system as well.

Site-specific amine-azide coupling reactions: The site-specific incorporation of the urea functional group proved to be independent of the alkylamine introduction site. See FIGS. 11A and 11B. In order to determine whether the reactivity of the alkylamine group is affected by its location on the DNA sequence, the reaction of a small DNA (TTTTT) with an alkylamino group on the internal thymidine (alkylamine on thymidine base), 3′ terminus, and 5′-terminus was studied. See FIG. 11A. Assessment of the modification was performed by using fluorophore-azide (1k, boron dipyrromethene, BODIPY) to compare the reaction efficiency of the different DNAs (see FIG. 11B), because the fluorescence intensity from the DNAs after the reaction reflects the efficacy of the reaction process. The DNAs were incubated with the reaction cocktails containing the fluorophore azide (1k) and triphenylphosphine, and then the reactions were subjected to the buffer exchange process to remove the labeling reagents and ionic liquid at a different time points (0, 30 and 60 min). The fluorescence intensities of the DNA after the buffer exchange were visualized on a nylon blot membrane. See FIG. 11A. There is a slight difference of the labeling efficiency tendency, but no substantial difference of the reactivity at any position was observed, and the negative control without amine groups displayed no meaningful reactivity. The similar observation was also confirmed by MALDI-MS analysis, and those results demonstrated the generality of the labeling process on different locations of alkylamines. Taking advantage of the minimal reactivity of the phosphine-azide toward endogenous DNA functional groups, we were able to selectively modify alkylamine-labeled DNA in a mixture with other untagged DNAs. See FIG. 11C. Thymidine pentamer (TTTTT) with and without the alkylamine tag at the 5′ terminus was incubated with BODIPY-azide (1k) and triphenylphosphine in the presence of a series of DNAs ranging from 10 to 300 base pairs. Agarose gel analysis showed a single fluorescence band at the bottom of the gel representing the alkylamine-containing condition. In contrast, a total stain of the gel with a SYBR® fluorophore confirmed the presence of a number of DNAs, demonstrating that the modification process indeed occurred preferentially on the alkylamine-tagged DNA over other untagged DNAs.

Preparation of DNA aptamer conjugates: The ionic liquid-based approach also enabled the introduction of a hydrophobic anchor onto a DNA aptamer. See FIGS. 12A-12D. Synthetic DNAs with hydrophobic tags have been studied increasingly as hydrophobic DNAs exhibit unique properties such as formation of nanostructures and cell permeability for molecular transport across cell membranes.^{96,97} However, due to the preference of an aqueous or highly polar solution of DNA with its polyionic nature, introduction of nonpolar, hydrophobic groups including lipids and steroids is chemically challenging. With the ionic nature of the hydrophobic scaffold of ionic liquids, it was hypothesized that the presently disclosed BINDRS strategy could address the dilemma of a reaction solvent incorporating a hydrophobic tag reacting with hydrophilic DNA molecules. A DNA aptamer toward the RNA hairpin of human immunovirus (HIV)-1 transactivation-responsive (TAR) element⁹⁸ was incubated with cholesterol azide (1l) and different types of phosphine reagents. See FIG. 12A. Consistent with the known cholesterol-induced aggregation of DNA⁹⁹ as well as with the phosphine screening experiments in FIGS. 10A-10D, the agarose gel analysis showed the aggregation of the DNAs with active triarylphosphine reagents in a high conversion but not from sterically bulky alkylphosphines nor from phosphine oxide negative control conditions. See FIG. 12B. Even though the polarity or solubility of DNAs would have been altered by the cholesterol modification processes, no significant loss of nucleotides was observed after the buffer exchange, as confirmed by results from the Mayer's hemalum stain. The high efficiency of the modification process with cholesterol was confirmed by MALDI-MS. See FIG. 12C. The aggregation observed in the agarose gel is indicative of the potential capability of the aptamer modified with a hydrophobic tag to diffuse into the cell membrane, and accordingly, the cholesterol-tagged aptamer was studied in confocal microscope experiments using a cancer cell line. Cultured HeLa cells were incubated with the cholesterol aptamer hybridized with its complementary sequence bearing a cyanine fluorophore (Cy5), and the fluorescence signal was visualized on a confocal microscope. See FIG. 12D. As anticipated,¹⁰⁰ the fluorescence signal derived from the Cy5 fluorophore was observed to be dependent on the modification. Staining of the actin filament with a phalloidin-fluorophore conjugate shows the presence of cells in both conditions. Together, these experiments demonstrate the successful introduction of cholesterol to the aptamer sequence through the ionic liquid-based reaction.

Finally, the ionic liquid-based amine-azide coupling was applied to labeling of a therapeutically important DNA aptamer. See FIGS. 13A and 13B. The Her2 receptor is an epidermal growth factor related protein (ErbB2) family of receptor tyrosine kinases and an emblematic example of overexpressed proteins in several types of cancer cells.¹⁰¹ See FIG. 13A. A sequence (42 nucleotides) of a DNA aptamer to the Her2 receptor was adapted from that originally reported by Yarden and co-workers.¹⁰² Modification of the Her2 aptamer with biotin-azide reagent proceeded smoothly in the same reaction conditions as those used with other aptamers, and was confirmed by MALDI-MS and anti-biotin southern blotting. A gel shift assay using streptavidin also demonstrated the consumption of the unmodified aptamer. The biotinylated aptamer was used for cell imaging experiments on a Her2-overexpressing cell line, SK-BR-3. Treatment of SK-BR-3 cells with the biotinylated aptamer and streptavidin-fluorophore (Cy5) conjugate displayed strong fluorescence signals while negligible fluorescence was observed from the unconjugated aptamer. See FIG. 13B. Of note, cells treated with the biotinylated aptamer hybridized with its complementary sequence showed substantial decrease in the fluorescence signals, highlighting the importance of the single-strand aptamer structure for its binding with the target protein. In addition, treatment of the cells with the Her2 aptamer prior to the introduction of the biotinylated aptamer caused substantial decrease of fluorescence response, and this competition experiment shows the binding of the modified aptamer to the same antigen target. As such, conservation of the aptamer's binding capability toward antigen targets even after treatment with ionic liquids and phosphine/azide reagents was confirmed, demonstrating the practical utility of the ionic liquid-DNA bioconjugation approach.

Accordingly, the ionic liquid-based urea-forming reaction has been successfully applied to the site-specific modification of unprotected DNA substrates. The high reaction efficiency at a desired location and high tolerance toward a variety of functional groups on azide and phosphine reagents could be of significant help in tailoring the technology to more specific applications. Thanks to the widespread use of azide-alkyne cycloaddition reactions in the chemistry and biology communities.¹⁰³ there are numerous commercially available alkylazide reagents, and the current work can be readily adopted for diverse applications. The shelf-stable nature of the alkylazide and triarylphosphine reagents would also be practically helpful in this context. Persistent issues of common amine-targeting reagents originate from the reagent instability such as the hydrolytic decomposition of N-hydroxysuccinimide (NHS) ester reagents for the acylation reaction and the aerobic oxidation of aldehyde reagents used in the reductive alkylation reaction. The presently disclosed ionic-liquid bioconjugation for nucleotide substrates provides access to untapped chemical labeling methodologies for preparation of nucleotide conjugates.

### Example 26

General Materials and Methods for Saccharide Bioconjugation Studies

All the chemicals including saccharides were purchased from commercial vendors unless otherwise noted. 1-Azido-2-(2-methoxyethoxy)ethane was synthesized according to previous reports.⁵⁷ Doxorubicin hydrochionde (D4193) and Disialylnonasaccharide-β-ethylazide (D4217) was purchased from Tokyo Chemical Industry (Tokyo, Japan) Valrubicin (AD-32) was purchased from Biovision (B1833. Biovision Inc., Milpitas, Calif., United States of America) and Vancomycin HCl was purchased from APExBIO (B1223. APExBio, Houston, Tex. United States of America). Oritavancin was purchased from Carbosynth (AA16180, Carbosynth Ltd., Staad, Switzerland). Toluidine blue (01804) and brilliant blue (6104-59-2) were purchased from Chem-Impex (Chem-Impex International. Wood Dale, Ill., United States of America) 7-Benzylamino-4-nitrobenz-2-oxa-1,3-diazole (NBD-amine) was purchased from Combi-Blocks (QA-8328, Combi-Blocks Inc., San Diego, Calif., United States of America). Hyaluronic acid (HA101) and hyaluronate azide (HA-1901) were purchased from CreativePEGWorks (Chapel Hill, N.C. United States of America). 5-TAMRA cadaverine (1248-25) and trans-cycloctyne (TCO)-amine HCl salt (1021-25) were purchased from ClickChemTools (Scottsdale, Ariz., United States of America). FITC labeled vancomycin (SBR00028-1.5MG), chitosan practical grade shrimp (417963-25G), DEAE-dextran hydrochloride (D9885-10g), fluorescein isothiocyanate dextran 200k (FD2000S), and amino-peg4-alkyne (764248-10MG) were all purchased from Sigma-Aldrich (St. Louis, Mo. United States of America).

NMR was performed on Bruker AVANCE NEO 500, 600, and 700 (Brucker, Billerica, Mass., United States of America). For ¹⁵N NMR, neat nitromethane (381.6 ppm, TCI N0209, Tokyo Chemical Industry. Tokyo. Japan) was used as the external standard. MALDI-MS was conducted on a Bruker Daltonics Autoflex-TOF (Brucker, Billerica, Mass., United States of America). A sample (0.5 or 1 μL) was mixed with an equal volume (0.5 or 1 μL) of matrix solution (20 mg/mL soln in 50:50:0.1 H₂O/MeCN/trifluoroacetic acid) on a ground-steel MALDI plate (Bruker 8280784, Brucker, Billenca, Mass., United States of America). Super-DHB or gentisic acid (DHBA) was used as a matrix.

LC-MS analysis of saccharides and small molecule models were performed on Shimadzu LCMS-2020 (Shimadzu, Kyoto, Japan) with a 2.6 μm C18 column (50×2.1 mm). The flow rate was 1 mL/min with the gradient of acetonitrile (5-90%) in the presence of 0.1% formic acid. The analysis of the reactions was performed by the UV detection saccharide at 280 nm. For the other saccharides (doxorubicin and vancomycin), LC-MS analysis was conducted on Agilent Technologies 1260 Infinity II series single quad instrument with 5 μm Luna C18 column (150×4.6 mm) (Agilent Technologies, Santa Clara, Calif., United States of America). The flow rate was 0.5 mL/min with the gradient of acetonitrile (10-90%) in the presence of 0.1% trifluoroacetic acid. The analysis of the reactions was performed by the UV detection of peptide peaks at 254 nm. LC-MS analysis of modified DNA was performed on Thermo Vanquish LC system and LTQ-XL linear ion trap MS system with a C18 column (HYPERSIL GOLD™ 25003-032130, particle size 3 μm, diameter: 2.1 mm, length: 30 mm; Thermo Fisher Scientific, Waltham, Mass., United States of America). The flow rate was 0.4 mL/min. Triethylammonium acetate buffer (5 mM, pH 7.2) was used as eluent with the gradient of acetonitrile (5-40% for 3.5 min, and then 90% for 1.5 min). The analysis of the reactions was performed by the UV detection at 254 nm.

Gel fluorescence and western blot imaging was performed on Amersham IMAGEQUANT® 800 (Cytiva, Upsala, Sweden). Gel or blot imaging was conducted using 360-nm, 535-nm, and 635-nm light sources with correlating emission bandpass filters at 525 nm (±20 nm), 605 nm (±40 nm), and 705 (±40 nm), relatively. Anti-biotin western blot was conducted with streptavidin-Cy5 conjugate (Jackson ImmunoResearch 016-170-084, 1:2,000 dilution (Jackson ImmunoResearch, West Grove, Pa., United States of America)) after blocking with 5% BSA in TBST buffer.

FT-IR was performed on Cary 630 FTIR spectrometer by Agilent Technologies (Santa Clara, Calif., United States of America).

Cell culture: HEK 293T cells were cultured in Dulbecco's Modified Eagle Medium (DMEM) with Glutamax, 10% fetal bovine serum (FBS) and penicillin/streptomycin (0.5 mg/mL) using 10 cm Petri dishes (Sigma-Aldrich 280721, Sigma-Aldrich, St. Louis, Mo., United States of America) under 5% CO₂ at 37° C.

### Example 27

### Post Reaction Clean-Up Processes for Ionic Liquid-Based Saccharide Bioconjugation

Extraction of hydrophilic saccharides: To BMPy OTF, potassium bicarbonate aqueous solution (20 mM final concn from 2-M stock solution), fluorescein isothiocyanate (FITC)-labeled dextran (1 mg/mL final concn from 5 mg/mL-stock solution) and 1× phosphate buffered saline (0.3 μL) was added (30 μL total volume) and subjected to liquid-liquid extraction using a water-immiscible ionic liquid (BMPy NTF₂, 30 μL) and H₂O (60 μL). FITC-dextran was recovered from the solution by transferring the aqueous layer to a new Eppendorf tube. Extraction was confirmed by visualization under a short UV wavelength (360 nm).

Acetone precipitation: To BMPy OTF, fluorescein isothiocyanate (FITC)-labeled vancomycin (3.0 mM final concn from 25-mM stock solution was added (30 μL total volume). Cold acetone (600 μL, −20° C.) was added in one portion to the mixture in a 1.7-mL Eppendorf tube. The mixture was mixed by flipping the tube upside-down multiple times and kept at ˜80′° C. for 1 h. Once removed from the freezer, the mixture was centrifuged (15,000 rcf, 15 min, 4° C.) to obtain the precipitates. Then, the acetone was removed and the pellet air-dried on bench at rt for 15 min

Extraction of hydrophobic saccharides: To EMIM OAc, 1× phosphate buffered saline (0.3 μL) and valrubicin (0.5 mM final concentration from 10-mM stock solution in DMSO) were added (30 μL total volume). The mixture was subjected to liquid-liquid extraction using a 2:1 mixture of ethyl acetate/water. Valrubicin was obtained from the mixture by transferring the aqueous layer to a new Eppendorf tube.

Thin Layer chromatography of small saccharides and molecules: To a 1:1 mixture of BMPy OTf/MeOH in a 1.7-mL Eppendorf tube, a mixture of 3:3:1 valrubicin/NBD-amine/brilliant blue (5 mM final concn from 50-mM stock solution of each) was added. The resulting solution (10 μL) was spotted on a reverse-phase TLC (#1156850001; MilliporeSigma, Burlington, Mass., United States of America) and developed using 70% MeOH in H₂O and 1% trifluoroacetic acid. The portion of C18/silica gel containing valrubicin was extracted using hexafluoroisopropanol (750 uL) by sonicating the mixture for 10 min. The mixture was centrifuged (15,000 rcf, 15 mins, room temperature) to obtain the supernatant containing valrubicin.

### Example 28

### General Procedure for Acetone Precipitation

Cold acetone (600-1200 μL, −20° C.) was added in one portion to the reaction mixture (typically 20-40 μL) in a 1.7-mL Eppendorf tube. The mixture was mixed by flipping the tube upside-down multiple times and kept at −80° C. for 1 h to overnight. Once removed from the freezer, the mixture was centrifuged (15,000 rcf, 15 min, 4′° C.) to obtain the precipitates. Then, the acetone was removed and the pellet air-dried on bench at rt for 15 min. For MALDI-MS analysis, the pellet was further washed with an additional cycle of acetone and centrifugation before air-drying process. The dried pellet was reconstituted in 10-40 μL of water and analyzed by respective analytical methods.

### Example 29

### Saccharide-Small Molecule Model Modification in Ionic Liquids

General Conditions: To ionic liquids (typically 10-40 μL for analytical scale), potassium bicarbonate aqueous solution (20 mM final concentration from 2-M stock solution in water), saccharide or small molecule (0.02-0.7 mM final concentration from 25-50-mM stock solution in water), alkyl azide (3-125 mM final concentration from 100-1000-mM stock solution in DMSO), and PAr₃ or O═PPh₃ (3-125 mM final concentration from 100-500-mM stock solution in DMSO) were added. The final concentration of H₂O was kept lower than 6% v/v. The reaction mixture was incubated at 37-50° C. for 2 h.

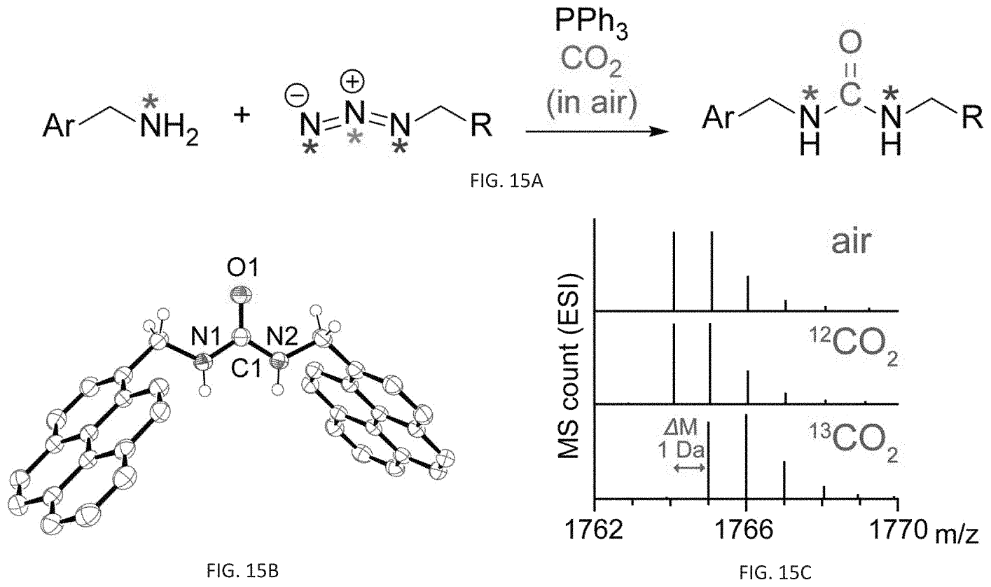

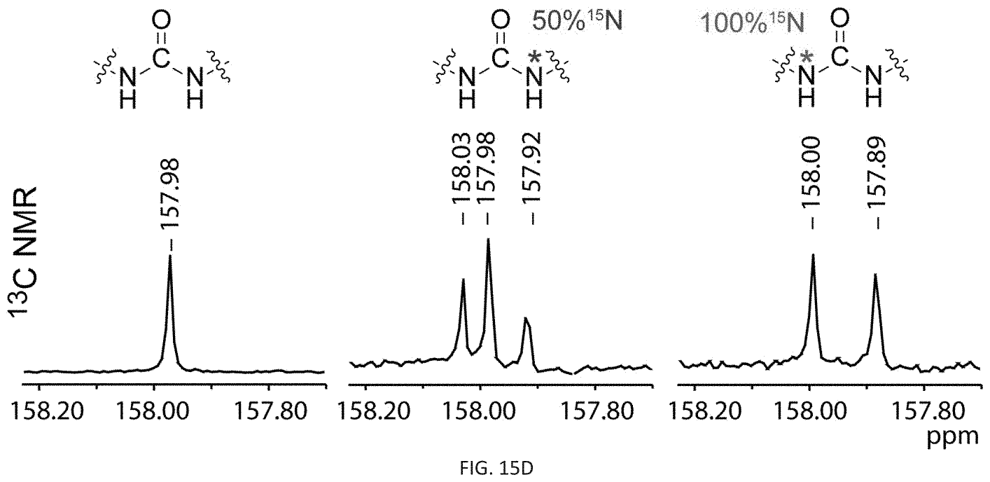

Pyrene amine modification with pyrene azide (a procedure for FIG. 15B): To DMF (30.25 uL), pyrene-amine (100 mM final concn from 0.4-M stock solution), pyrene-azide (20 mM final concn from 1-M stock solution), and PPh₃ (12.5 mM final concn from 1-M stock solution in 1:1 toluene/DMSO) were added (50 μL total volume). The reaction was quadruplicated in different Eppendorf tubes. The reaction mixtures were incubated at 50° C. overnight. The reaction mixtures were combined into one Eppendorf tube and placed at −20° C. for 1 week to afford crystals suitable for the X-ray analysis.

Pentanucleotide modification with azide: The urea forming reaction on pentanucleotide (5′-TTTTT-3′ where T=thymidine with alkylamine containing 12 carbon linker) was performed following the same general modification procedure as described in above in 30-μL scale with the following conditions. 5′-TTTTT-3′ (0.2 mM final concentration from 5-mM stock solution in H₂O), ¹²C/¹³C—K₂CO₃ (20 mM final concentration from 2-M stock solution in H₂O), azide 2 (7.5 mM final concentration from 250-mM stock solution in DMSO), and PPh₃ (20 mM final concentration from 500-mM stock solution in DMSO) in BMPy OTf at 50° C. for 2 h. The product was purified by adding cold acetone (600 μL, −20° C.) to the reaction mixture, mixed by upside-down shaking and allowed to sit at −80° C. overnight. The precipitates were collected by centrifugation (15,000 rcf, 15 min, 4° C.), and acetone was removed. The pellet obtained was further washed with acetone and the centrifugation process repeated before the pellet was air-dried on the bench at room temperature for 15 min. The dried pellet was reconstituted in 30 μL of ammonium bicarbonate (NH₄HCO₃) aqueous solution (5 mM) and analyzed using LC-MS.

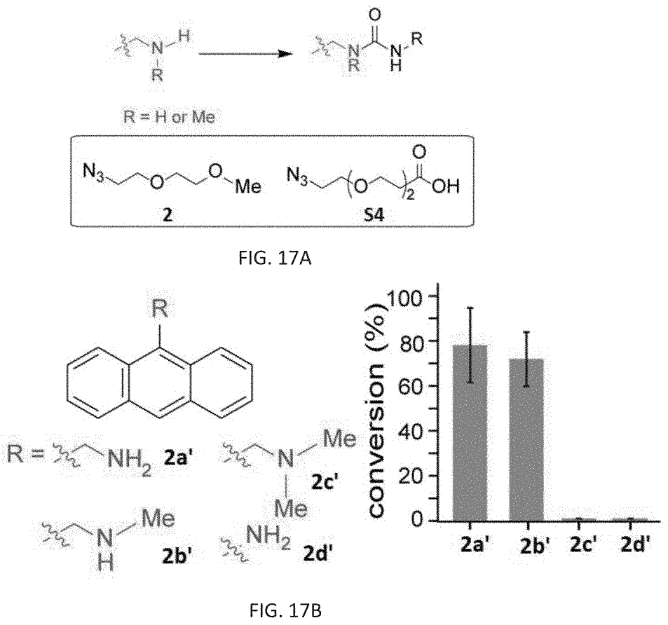

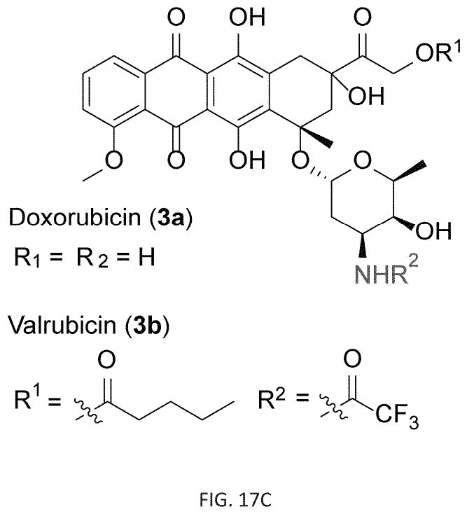

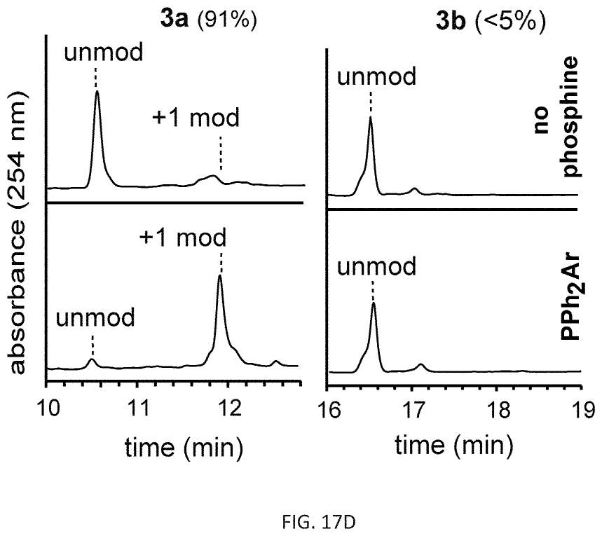

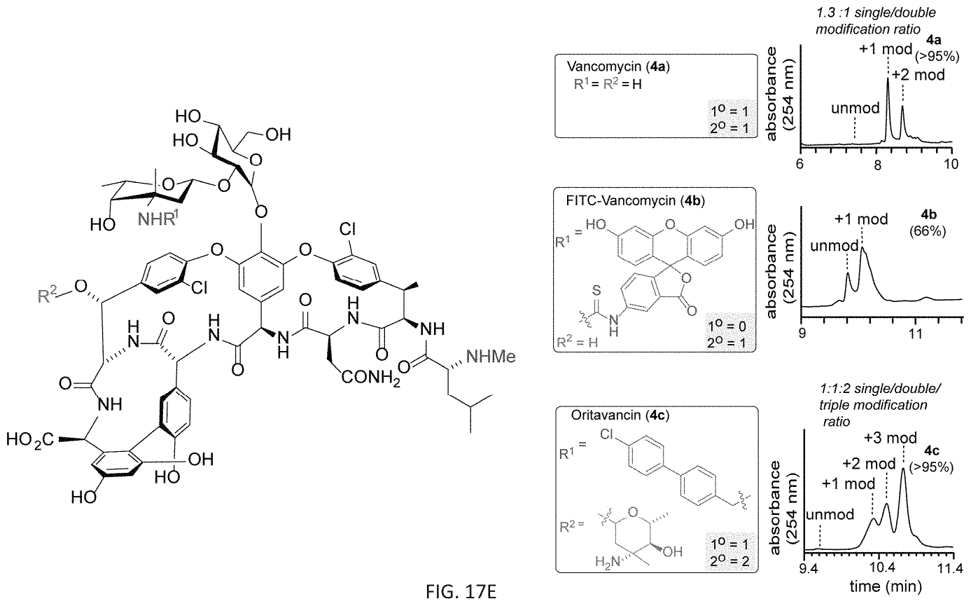

Reaction of amine-containing anthracene compounds (a procedure for FIG. 17B): To a 1:1:1 mixture of DMF (10.53 uL), MeCN (10.53 uL), and BMPyOTf (10.53 uL), potassium bicarbonate aqueous solution (20 mM final concn from 2-M stock solution), anthracene derivatives (2.5 mM final concn from 50-mM stock solution in DMSO). 2 (50 mM from 1000-mM stock solution in DMSO), and PPh₃ (50 mM final concn from 500-mM stock solution in DMSO) were added. The final concn of H₂O was kept at 1% v/v. The reaction mixture was incubated at a 50° C. incubator for 2 h. The reaction mixture was purified by thin layer chromatography (basic aluminum oxide 60, #1057130001, MilliporeSigma, Burlington, Mass., United States of America) with 98:2 dichloromethane/MeOH as eluents. The product was recovered from alumina by addition of MeCN (750 uL) and sonication. This mixture was centrifuged (15,000 rcf, 15 min, rt) and the supernatant was analyzed by LC-MS.

Doxorubicin/valrubicin modification (a procedure for FIG. 17D): Aqueous solution of potassium bicarbonate (20 mM final concentration prepared from 2-M stock solution in water), doxorubicin or valrubicin (0.3 mM final concentration prepared from 25-mM stock solution in DMSO), azide 2 (7.5 mM final concentration from 250-mM stock solution in DMSO) and PPh₂ (m-sulfophenyl) (3 mM final concentration prepared from 100-mM stock solution in DMSO) or DMSO used as a negative control were added to a 1:3 mixture of BMPy OTF and MeCN. The reaction mixture was incubated at 37° C. for 2 h. The product was purified by reverse-phase TLC plate (MilliporeSigma #11156850001; MilliporeSigma, Burlington, Mass., United States of America) using 95:5 acetonitrile/water. The portion of silica/C18 containing the desired saccharides was extracted using hexafluoroisopropanol (750 uL) by sonicating the mixture of the solvent and silica/C18 for 10 min. The mixture was centrifuged (15,000 rcf, 15 mins at room temperature), and the supernatant was transferred to a 5-mL Eppendorf tube. The silica/C18 was washed with additional HFIP (200 uL) and transferred to the same Eppendorf tube. The content of the tube was diluted with MeCN (3 mL), syringe filtered, and the solvent was evaporated by gentle flow of the nitrogen gas. The obtained solution was analyzed by LC-MS.

Modification of vancomycin and its derivatives: FITC-vancomycin and oritavancin. (a procedure for FIG. 17E): To BMPy OTf, potassium bicarbonate aqueous solution (20 mM final concn from 2-M stock solution), vancomycin and its derivatives (0.5 mM final concn from 25-mM stock solution in DMSO), azide S4 (125 mM final concn from 1000-mM stock solution in DMSO), and tritolylphosphine (125 mM final concn from 500-mM stock solution in 1:1 DMSO/toluene) were added (30 μL total volume). The reaction mixture was incubated at 50° C. for 2 h and subjected to acetone precipitation as described in the General procedure fir acetone precipitation (see Example 28) using 600 μL cold acetone. The reconstituted samples were analyzed using LC-MS.

### Example 30

### Chitosan Modification

To EMIM OAC (20 μL), chitosan or DEAE-dextran (1 mg/mL final concentration from 10 mg/mL stock solution in 1:1 BMIM: 1-M acetate buffer), biotin-peg3-azide (0.3-7.5 mM final concentration from 15-375 mM stock solutions in DMSO), and PPh₃ or O═PPh₃ (0.3-7.5 mM final concentration from 15-375 mM stock solution in DMSO) were added. The final concn of H₂O was kept lower than 6% v/v. The reaction mixture was incubated in a 37° C. incubator for 2 h and subjected to the following post-reaction cleanup process.

Post-reaction cleanup process: To the reaction mixture (20 μL) in a 1.7-mL Eppendorf tube, a mixture of 5:1 acetone/methanol (600 μL) was added in one portion. The mixture was mixed by upside-down shaking and sit at −80° C. for 1 h or overnight. The precipitates were collected by centrifugation (15,000 rcf, 15 min, 4° C.), and acetone/methanol was removed. The pellet was air-dried on the bench at room temperature for 15 min. The dried pellet was reconstituted in 20 μL of acetate buffer (20 mM, pH 5) and analyzed by dot blot methods.

Immunodetection of biotin tag of chitosan using anti-biotin antibodies: The reconstituted samples (0.5 μL) were heated for 1 min at 95° C. and then spotted onto nitrocellulose membrane. Eosin Y solution (0.1 mM final concentration in water from 50-mM stock solution) was used for the total stain purpose for 5 min, and the membrane was rinsed with water twice. The stained membrane was imaged by IMAGEQUANT® 800 (Cytiva. Upsala, Sweden) to obtain the colorimetric image. Then, the membrane was washed twice with TBST buffer for 5 min, blocked with 5% BSA in TBST buffer at rt for 20 min, incubated with streptavidin-Cy5 conjugate (1:2000) in the blocking buffer at rt for 40 min, washed with TBST buffer three times, and imaged by IMAGEQUANT® 800 (Cytiva. Upsala, Sweden).

### Example 31

### Pyrine-Azide Modification with Amine

To a 1:8:1 mixture of DMF (4.52 uL), MeCN (36.16 uL), and BMPyOTf (4.52 uL), potassium bicarbonate aqueous solution (20 mM final concn from 2-M stock solution), azidomethyl-pyrene (12.5 mM final concn from 250-mM stock solution in DMSO). 2-(2-methoxyethoxy)ethanamine (125 mM final concn from 1000-mM stock solution in DMSO), and PPh₃ (125 mM final concn from 500-mM stock solution in DMSO) were added. This reaction was replicated in 14 separate 1.7-mL Eppendorf tubes. The reaction mixtures were incubated at 50-° C. incubator for 1 h. Once the reaction was complete, all the mixtures were combined into one tube. The solution was purified by thin layer chromatography (basic aluminum oxide 60, #1057130001, MilliporeSigma. Burlington, Mass., United States of America) with 80:20 dichloromethane/hexane as eluents. This process was repeated two more times with 99:1 dichloromethane/MeOH as eluents for thin layer chromatography. The product was recovered from the alumina by addition of 9:1 MeCN/MeOH and sonication and analyzed by LC-MS and ¹H NMR after separating the insoluble alumina by centrifugation.

### Example 32

### Saccharide-Azide Modification with Alkylamine

BMPy OTf (16.8 uL), KHCO₃ (40 mM final concn from 2-M stock solution), DSNS-azide (0.2 mM final concn from 5-mM stock solution), alkyl amines with different degrees of substitution (20 mM final concn from 250-mM stock solution), and PPh; or O═PPh₃ (20 mM final concn from 1-M stock solution in 1:1 DMSO/toluene) were mixed and incubated at 50° C. for 2 hr. The rest of the procedure is the same as the acetone precipitation procedure from General procedure for acetone precipitation (see Example 28).

### Example 33

### Modification of Hyaluronic Acid Derivatives

In a typical procedure, to EMIM OAc (20 μL), hyaluronic acid derivatives hyaluronic acid derivatives (2 mg/mL final concentration from 40 mg/mL-stock solution in H₂O), amine reagent (10-20 mM final concentration from 250-mM stock solutions in DMSO), and PPh₃ or O═PPh₃ (20 mM final concentration from 250-mM stock solution in DMSO) were added. The final concn of H₂O was kept lower than 6% v/v. The reaction mixture was incubated in a 37° C. incubator for 2 h and subjected to precipitation before analysis.

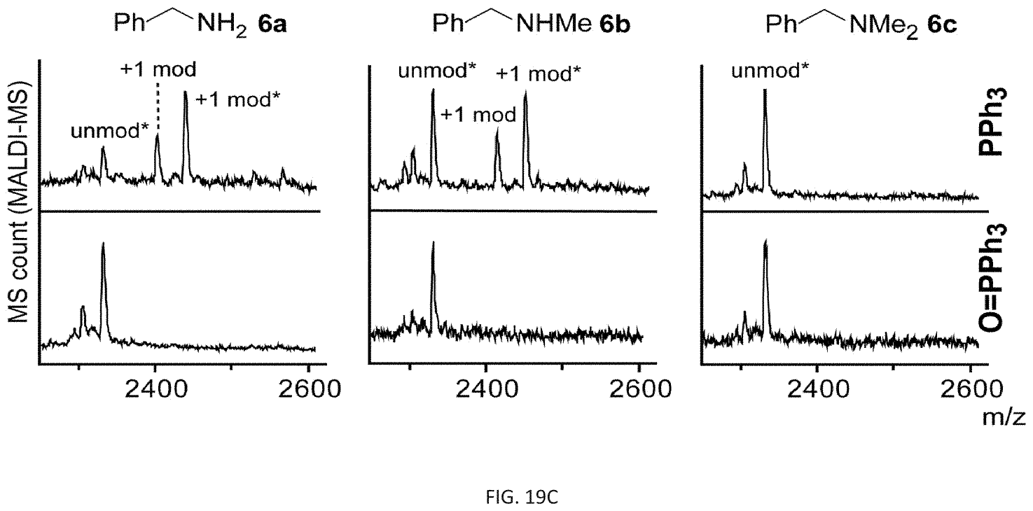

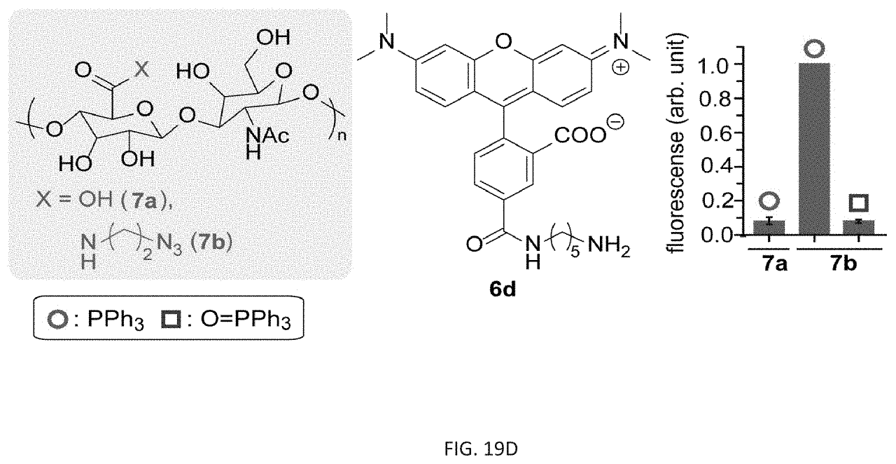

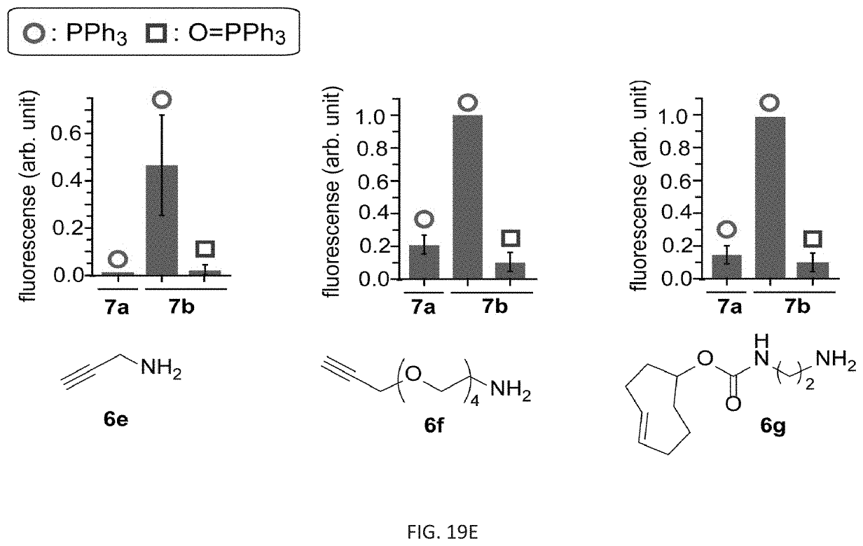

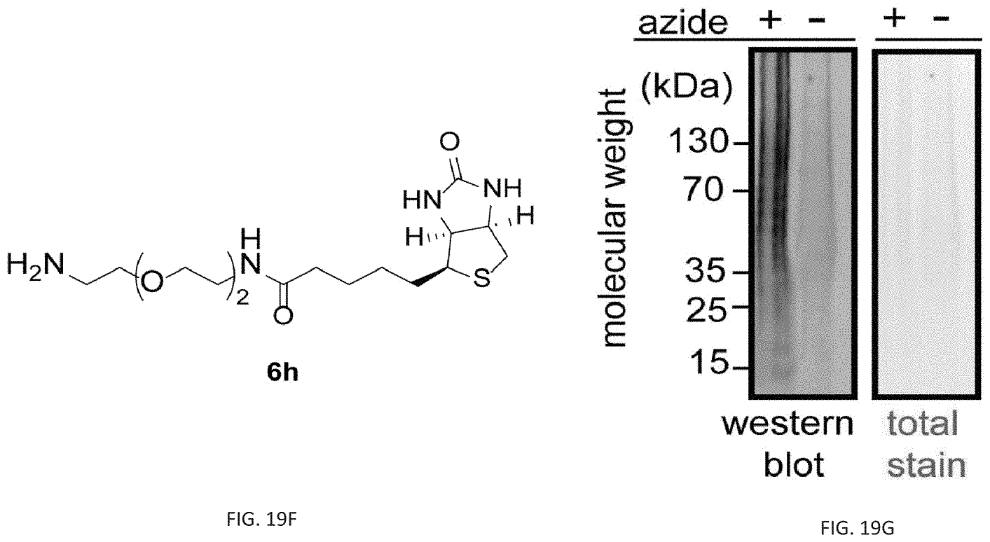

Hyaluronic acid derivative modification with TAMRA amine (a procedure for FIG. 19)): The modification procedure follows the typical procedure in 20 μL scale with following conditions. Hyaluronic acid derivatives (2 mg/mL final concentration from 40-mg/mL stock solution in H₂O), TAMRA-NH₂ (20 mM final concentration from 250-mM stock solution in DMSO), KHCO₃ (20 mM final concentration from 2-M stock solution in H₂O), and PPh₃ or O═PPh₃ (20 mM final concentration from 500-mM stock solution in DMSO) were added into EMIM OAC and incubated for 2 h at 37° C. To the reaction mixture (20 μL) in a 1.7-mL Eppendorf tube, a 5:1 mixture of cold acetone/methanol (600 μL) was added in one portion, mixed by upside-down shaking and placed overnight at −80° C. The precipitates were collected by centrifugation (15,000 rcf, 15 min, 4° C.), and acetone/methanol was removed, and the pellet was air-dried on the bench at room temperature for 15 min. The dried pellet was reconstituted with 20 μL H₂O, spotted onto nylon membrane (#11209299001, MilliporeSigma, Burlington, Mass. United States of America) (and washed overnight with 1:1 MeOH/DMSO, and the fluorescence intensity was quantified by IMAGEQUANT® 800 (Cytiva, Upsala. Sweden).

The reconstituted samples were also spotted separately on a new membrane and stained with diluted Toluidine Blue solution (3 mM final concentration in water from 250-mM stock solution in DMSO) for 5 min and rinsed 4 times with water. The stained membrane was imaged by IMAGEQUANT® 800 (Cytiva, Upsala. Sweden) to obtain the colorimetric image. For the quantification purpose, the experiment was done in triplicates but on different days and the fluorescence intensity was quantified by ImageJ software.

Hyaluronic acid derivative modification with Propargyl-NH₂ and Alkyne-Peg4-NH₂ (a procedure for FIG. 19E): The modification procedure follows the typical procedure for modification of hyaluronic acid derivatives above. Hyaluronic acid derivatives (2 mg/mL final concentration from 40-mg/mL stock solution in H₂O), propargyl amine or alkyne-peg4-amine (20 mM final concentration from 250-mM stock solution in DMSO), KHCO₃ (20 mM final concentration from 2-M stock solution in H₂O), and PPh₃ or O═PPh₃ (20 mM final concentration from 500-mM stock solution in DMSO) were added into EMIM OAc and incubated for 2 h at 37° C. To the reaction mixture (20 μL), in a 1.7-mL Eppendorf tube, a mixture of acetone/methanol was added in one portion. The precipitation was performed with cold acetone/methanol (5:1 ratio, 600 μL, −20° C.). After the addition of acetone/methanol, the mixture was mixed by upside-down shaking and sit at −80° C. overnight. The precipitates were collected by centrifugation (15,000 rcf, 15 min, 4° C.), and acetone/methanol was removed and the pellet was air-dried on the bench at room temperature for 15 min. The dried pellet was reconstituted with 7 μL H₂O, spotted onto nylon membrane and rinsed with MeOH for 5 min, then membrane was washed with water twice and subjected to chemical blotting^{[2]} for 30 min with the following conditions: THPTA (0.1 mM final concentration from 0.1-M stock solution in H₂O, sodium ascorbate (1.5 mM final concentration from 0.1-M stock solution in H₂O, CuSO₄ (0.1 mM final concentration from 100 mM stock solution in H₂O), and coumarin azide (10 μM final concentration from 20-mM stock solution in DMSO) in 5 ml of 1:1 H₂O/DMSO.

The reconstituted samples were also spotted separately on new nylon membrane and stained with the toluidine Blue solution (3 mM final concentration in water from 250-mM stock solution in DMSO) for 5 min, and the membrane was rinsed 4 times with water. The stained membrane was imaged by IMAGEQUANT® 800 (Cytiva, Upsala. Sweden) to obtain the colorimetric image. For the quantification purpose, the experiment was done in triplicates but on different days and the fluorescence intensity was quantified by ImageJ software.

Modification of hyaluronic acid derivatives with TCO-NH₂ (a procedure for FIG. 19E): The modification procedure follows the typical procedure for modification of hyaluronic acid derivatives above. Hyaluronic acid derivatives (2 mg/mL final concentration from 40-mg/mL stock solution in H₂O) TCO-NH₂ (10 mM final concentration from 250-mM stock solution in DMSO), KHCO₃ (20 mM final concentration from 2-M stock solution in H₂O), and PPh₃ or O═PPh₃ (20 mM final concentration from 500-mM stock solution in DMSO) were added into EMIM OAC and incubated for 2 h at 37° C. To the reaction mixture (20 μL), a mixture of acetone/methanol was added in one portion. The precipitation was performed with cold acetone/methanol (5:1 ratio, 600 μL, −20° C.). After the addition of acetone/methanol, the mixture was mixed by upside-down shaking and kept at −80° C. overnight. The precipitates were collected by centrifugation (15,000 rcf, 15 min, 4° C.), and acetone/methanol was removed and the pellet was air-dried on the bench at room temperature for 15 min. The dried pellet was reconstituted with 50-mM MES buffer containing sulfu-Cy5 tetrazine (20 μL, 1 mM) and incubated for 2 h at rt. Acetone (600 μL) was added to the reconstituted samples and precipitation was performed again at −80° C. overnight. After the centrifugation and removal of the supernatant, the samples were washed by two additional cycles of acetone addition and centrifugation. The pellet was air-dried on bench at rt for 15 min after removing the final acetone solution and then reconstituted with 20 μL H₂O, spotted on nylon membrane. The membrane was washed 4 times with 1:1 MeOH:DMSO and imaged by IMAGEQUANT® 800 (Cytiva, Upsala, Sweden) to obtain fluorescence image.

The reconstituted samples were spotted separately on a new nylon membrane and stained with the toluidine blue solution (3 mM final concentration in water from 250-mM stock solution in DMSO) for 5 min and rinsed 4 times with water. The stained membrane was imaged by IMAGEQUANT® 800 (Cytiva, Upsala, Sweden) to obtain the colorimetric image. For the quantification purpose, the experiment was done in triplicates but on different days and the fluorescence intensity was quantified by ImageJ software.

### Example 34

### Cell Lysis Studies

HEK 293T cultured cells without poly-D-lysine coating at 100% confluency (15 million) were taken out, washed three times with PBS, separated from the buffer by centrifugation (1000 rcf, 3 min, 4° C.), placed in −80° C. for 30 min, and lysed in PBS buffer (960 μL) containing 0.1% SDS, 0.1% triton and EDTA-free protease inhibitor (complete tablets, Roche #04-693-159-001 Roche Holding AG, Basel, Switzerland). The cell lysate was transferred to 1.7-mL Eppendorf tube, and a homogenizer was used to assure the complete lysis of cells. The cell lysate was placed for 30 min in ice before centrifugation (15000 rcf, 15 min, 4° C.) and the supernatant was taken out to run the oxidation reaction.

Periodate oxidation and hydrazone formation on saccharide groups of glycoproteins in cell lysates: Sodium periodate (30 mM final concentration from 1000-mM stock solution in H₂O) or H₂O (as a negative control) was added to a 1.7-mL Eppendorf tube containing cell lysates (310 μL total). The mixture was incubated for 1 h at rt before quenching with 20% v/v glycerol in TBST buffer (16.5 μL). 103-μL aliquots were transferred to three different tubes, and cold acetone (1200 μL, −20° C.) was added to each tube. After the addition of acetone, the mixture was mixed by inversion processes and kept at −80° C. overnight. The precipitates were collected by centrifugation (15,000 rcf, 15 min, 4° C.), acetone was removed, and the pellet was air-dried on the bench at room temperature for 15 min. The dried pellet was reconstituted with 50 μL of complete PBS buffer (0.1% v/v SDS, 0.1% v/v triton and EDTA-free protease inhibitor) followed by the addition of azido-PEG4-hydrazide (HY-140814; MedChemExpress, Monmouth Junction, N.J., United States of America) (12 mM final concentration from 20-mM stock solution in DMSO) to both oxidized and control lysates. After an overnight reaction, acetone (600 μL) was added to the resultant mixture which was put at −80° C. overnight. Centrifugation (15,000 rcf, 15 mins, 4° C.) afforded the hydrazone azide pellets which were air-dried at rt for 15 min and reconstituted in 12.5-μL PBS buffer (0.1% v/v SDS, 0.1% v/v triton). The lysate concentration was determined by Bradford assay, and the concentration of the azide-tagged and non-tagged solutions were adjusted to the same.

Modification of azide containing saccharides in cell lysates. (a procedure for FIG. 19G): To BMPy OTf (16.4 μL), KHCO₃ (20 mM final concentration from 2-M stock solution), cell lysates with and without hydrazone azide (0.91 mg/mL), biotin amine (20 mM final concentration from 250-mM stock solution), and PPh₃/O═PPh₃ (20 mM final concentration from 500-mM stock solution in DMSO) were added. The final concn of H₂O was kept lower than 6% v/v. The reaction mixture was incubated at 37° C. for 2 h and subjected to overnight precipitation using 1:1 toluene/acetone (600 μL) at −80° C. The resultant suspension was centrifuged (15,000 rcf, 15 mins, 4° C.), the air-dried pellet was reconstituted in 5 μL PBS buffer (0.1% v/v SDS, 0.1% v/v triton). To the reconstituted pellet (5 μL), water (2.5 μL) and 4×LDS sample buffer (2.5 μL) were added, mixed and used for SDS-PAGE gel electrophoresis for 40 min. The modified glycoproteins on the gel were transferred to a PVDF membrane (Biorad TransBlot Turbo PVDF membrane L002048A, Bio-Rad Laboratories, Hercules, Calif. United States of America). The membrane was then activated with methanol and washed twice with water before Ponceu staining to obtain the colorimetric image using IMAGEQUANT® 800 (Cytiva, Upsala, Sweden). The membrane was once again reactivated with methanol and washed twice with water followed by TBST buffer for 5 mins, blocked with BSA (50 mg/mL) in TBST buffer at rt for 1 h, incubated with streptavidin-Cy5 conjugate (1:2000) in the blocking buffer at rt for 40 min, washed with TBST buffer three times, and imaged by IMAGEQUANT® 800 (Cytiva, Upsala, Sweden). Molecular weight marker (Thermo Scientific 26619: Thermo Fisher Scientific, Waltham, Mass., United States of America) was used for the analysis.

### Example 35

### Preparative Synthesis of Small Molecules

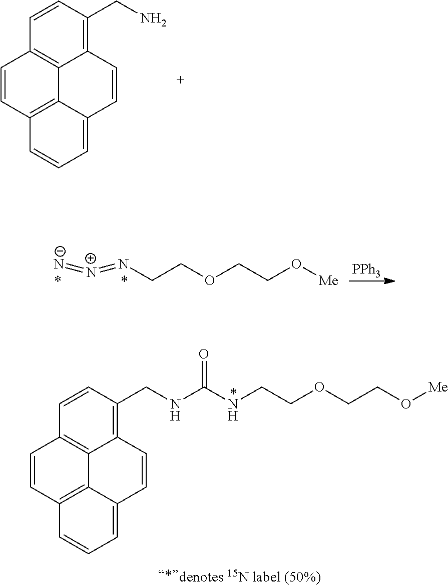 [SMILES: NCc1ccc2ccc3cccc4ccc1c2c34 ; COCCOCCN=[N+]=[N-] ; COCCOCCNC(=O)NCc1ccc2ccc3cccc4ccc1c2c34 ; "PPh3"]

Synthesis of pyrene urea compound using alkylamine and terminal ¹⁵N-enriched azide: Alkyl azide (25 mg, 0.165 mmol) was added to a 4-mL vial equipped with a magnetic stir bar. DMF (280 uL), DMSO (100 uL), and K₂CO₃ aq solution (13.6 uL, 2M, 0.034 mmol) were added. To the mixture. (aminomethyl)pyrene hydrochloride (9.1 mg, 0.039 mmol) was added. The mixture was heated for 5 min at 50° C., and then PPh₃ (90.2 mg, 0.327 mmol) solution in DMF (300 uL) was added dropwise. After the reaction mixture was heated at 50° C. overnight, the formation of the product was confirmed and purified by TLC (2:1 hexane/dichloromethane on basic aluminum oxide 60, #1057130001 (MilliporeSigma, Burlington, Mass., United States of America). The product was recovered from the alumina by addition of methanol (1 mL) and sonication for 3 minutes after which MeCN (9 mL) was added and sonicated for another 3 min. The suspension was passed through CELITE® followed by paper filtration to remove CELITE®. After concentrating the filtrate by nitrogen flow, the resulting solid was reconstituted in 1:1 MeOH/MeCN. TLC and CELITE® process was repeated using 99:1 dichloromethane/methanol (R_{f}=0.3-0.4), and removal of the volatiles under vacuum afforded the pyrene urea compound as off-white solids (4.3 mg, 17%). ¹H NMR (700 MHz, DMSO-d₆): δ 8.40 (d, J=9.2 Hz, 1H), 8.31 (dd, J=11.9, 7.5 Hz, 2H) 8.25 (m, 2H), 8.15 (s, 2H), 8.07 (t, J=7.6 Hz, 1H), 8.02 (d, J=7.6 Hz, 1H), 6.67 (br, 1H), 6.04 (br, 1H), 4.94 (d, J=5.9 Hz, 2H), 3.49 (m, 2H), 3.40 (m, 5H), 3.21 (m, 5H). ¹⁵N {¹H} NMR (60 MHz, CD₃CN/DMSO-d₆ (95:5). ¹⁵N-enriched sample): δ 77.2.

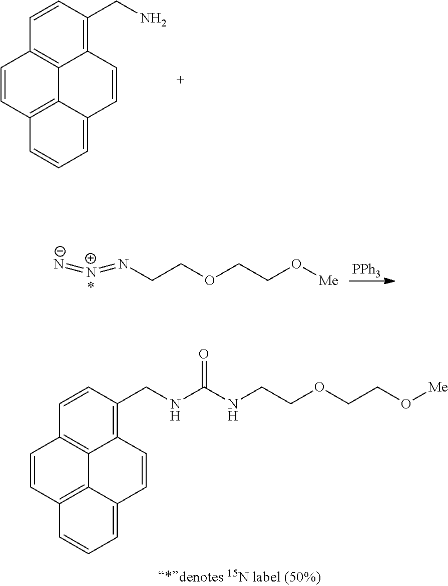 [SMILES: NCc1ccc2ccc3cccc4ccc1c2c34 ; COCCOCCN=[N+]=[N-] ; COCCOCCNC(=O)NCc1ccc2ccc3cccc4ccc1c2c34 ; "PPh3"]

Synthesis of pyrene urea compound using alkylamine and internal ¹⁵N-enriched azide: Azide (25 mg, 0.165 mmol) was added to a 4-mL vial equipped with a magnetic stir bar. DMF (280 uL), DMSO (100 uL), and K₂CO₃ aq solution (13.6 uL, 2M, 0.034 mmol) were added. To the mixture, (aminomethyl)pyrene hydrochloride (9.1 mg, 0.039 mmol) was added. The mixture was heated for 5 min at 50° C., and then PPh₃ (90.2 mg, 0.327 mmol) solution in DMF (300 uL) was added dropwise. After the reaction mixture was heated at 50° C. overnight, the formation of the product was confirmed and purified by TLC (2:1 hexane/dichloromethane on basic aluminum oxide 60, #1057130001 (MilliporeSigma, Burlington, Mass., United States of America). The product was recovered from alumina by addition of methanol (1 mL) and sonication for 3 minutes after which MeCN (9 mL) was added and sonicated for another 3 min. The suspension was passed through CELITE® followed by paper filtration to remove CELITE®. After concentrating the filtrate by nitrogen flow, the resulting solid was reconstituted in 1:1 MeOH/MeCN. TLC and CELITE® process was repeated using 99:1 dichloromethane/methanol (R_{f}=0.3-0.4), and removal of the volatiles under vacuum afforded the pyrene urea compound as off-white solids (3.6 mg, 14%). ¹H NMR (700 MHz, CD₃CN/DMSO-d₆ (95:5): δ 8.48 (d, J=10.8 Hz, 1H), 8.34 (t, J=7.7 Hz, 2H) 8.30 (d, J=7.1 Hz, 1H), 8.28 (d, J=8.8 Hz, 1H), 8.19 (s, 2H), 8.14 (t, J=8.9 Hz, 1H), 8.03 (d, J=8.9 Hz, 1H), 6.16 (br, 1H), 5.60 (br, 1H), 5.08 (d, J=6.8 Hz, 2H), 3.60 (m, 2H), 3.54 (t, J=6.4, 2H), 3.50 (m, 2H) 3.36 (m, 2H), 3.32 (s, 3H). ¹⁵N {¹H}NMR (60 MHz, CD₃CN/DMSO-d₆ (95:5). ¹⁵N-enriched sample): δ 244.78.

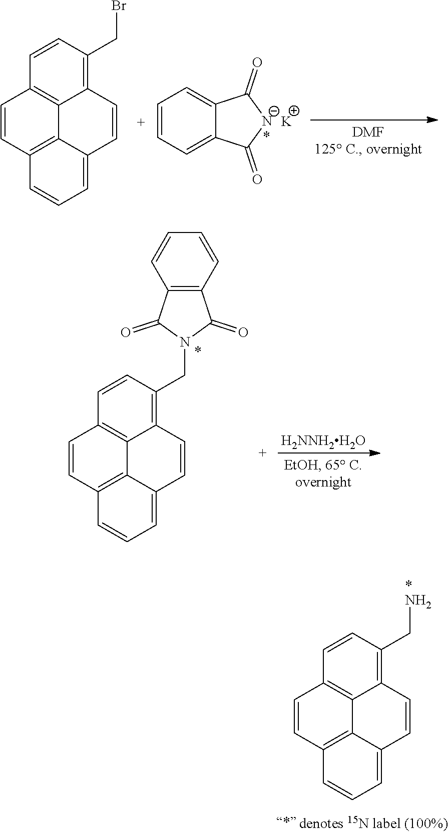

Synthesis of ¹⁵N-enriched pyrene amine: The synthetic procedure was adopted from previous reports using ⁵N-enriched potassium phthalimide (#299243; MilliporeSigma, Burlington, Mass., United States of America).¹²⁵ Pyrene bromide (48.6 mg, 0.165 mmol) was dissolved in DMF (350 uL) in a 4 mL vial equipped with a magnetic stir bar. To the mixture, ⁵N-enriched potassium phthalimide (50 mg, 0.165 mmol) was added, the reaction vial was sealed with cap and vinyl tape and covered with aluminum foil. After the reaction mixture was heated overnight at 125° C. cold H₂O (2 mL) was added to the resulting mixture and transferred to a 0.1.7 mL eppendorf tube. The precipitates were collected by centrifugation (15,000 rcf, 15 mins, 4° C.), the pellets were washed again with H₂O and centrifuged using the same conditions as stated previously. Evaporation of the volatiles by gentle flow of nitrogen gas afforded the phthalimide intermediate. The ⁵N-enriched pyrene phthalimide intermediate was dissolved in EtOH (2 mL) in a 4 mL vial, and hydrazine hydrate (54.6 mg, 1.09 mmol) was added. After the reaction mixture was heated overnight at 65° C. EtOH (2×2 mL) was added to the resulting mixture which was then centrifuged (15,000 rcf, 15 mins, 4° C.) to obtain the supernatant. A gentle flow of nitrogen gas over the supernatant gave the product as a mixture with phthalimide-derived impurity (28.5 mg). This crude material was used without further purification ¹H NMR (500 MHz, DMSO-d₆): δ 8.41-8.06 (m, 9H), 4.47 (s, 2H).

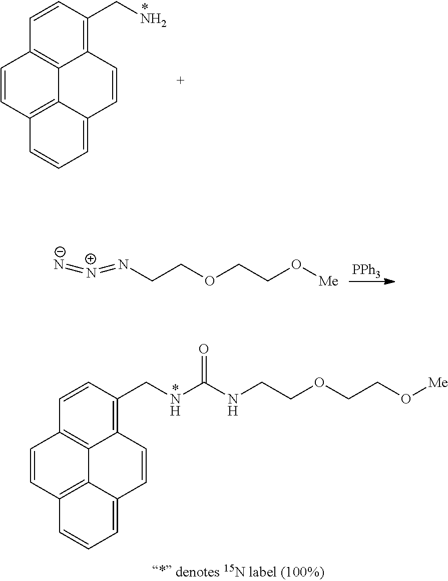 [SMILES: NCc1ccc2ccc3cccc4ccc1c2c34 ; COCCOCCN=[N+]=[N-] ; COCCOCCNC(=O)NCc1ccc2ccc3cccc4ccc1c2c34 ; "PPh3"]

Synthesis of pyrene urea compound using ¹⁵N enriched alkylamine and unlabeled azide: Azide (25 mg, 0.165 mmol) was added to a 4-mL vial equipped with a magnetic stir bar. DMF (280 uL), DMSO (100 uL), and K₂CO₃ aq solution (13.6 uL, 2M, 0.034 mmol) were added. To the mixture, ¹⁵N enriched aminomethyl pyrene (9.1 mg, 0.039 mmol) was added. The mixture was heated for 5 min at 50° C., and then PPh₃ (90.2 mg, 0.327 mmol) solution in DMF (300 uL) was added dropwise. After the reaction mixture was heated at 50° C. overnight, the formation of the product was confirmed by TLC, and the reaction mixture was also purified by TLC (2:1 hexane/dichloromethane on basic aluminum oxide 60, (#1057130001; MilliporeSigma, Burlington, Mass., United States of America). The product was recovered from the alumina by addition of methanol (I mL) and sonication for 3 minutes, and then MeCN (9 mL) was added and sonicated for another 3 min. The suspension was passed through CELITE® followed by paper filtration to remove CELITE®. After concentrating the filtrate by nitrogen flow, the resulting solid was reconstituted in 1:1 MeOH/MeCN. TLC and CELITE®, process was repeated using 99:1 dichloromethane/methanol (R_{f}=0.3-0.4), and removal of the volatiles under vacuum afforded the pyrene urea compound as off-white solids (2.2 mg, 9%). ¹H NMR (500 MHz, DMSO-d₆): δ 8.41 (d, J=9.3 Hz, 1H), 8.31 (t, J=8.2 Hz, 2H) 8.26 (t, J=7.3 Hz, 2H), 8.16 (s, 2H), 8.08 (t, J=7.6 Hz, 1H), 8.03 (d, J=7.6 Hz, 1H), 6.69 (br, 1H), 6.07 (br, 1H), 4.96 (d, J=5.6 Hz, 2H), 3.51 (m, 2H), 3.41 (m, 3H), 3.21 (m, 4H). ¹⁵N {¹H} NMR (60 MHz, DMSO-d₆, ¹⁵N-enriched sample): δ 83.51. ESI-MS: calcd for C₂₃H₂₅N₂O₃ [M+H]⁺ 378, found 378.

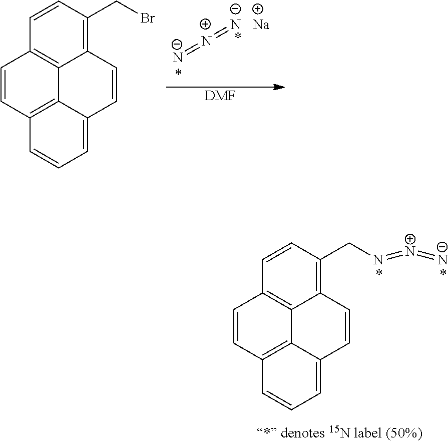

Synthesis of ¹⁵N-enriched pyrene azide: The synthetic procedure was adopted from previous reports using ¹⁵N-enriched sodium azide (#609374; MilliporeSigma, Burlington, Mass., United States of America).¹²⁶ Pyrene bromide (50.8 mg, 0.172 mmol) was added to DMF (300 uL) in a 4-mL vial equipped with a magnetic stir bar. To the mixture, powder of NaN₃ (17.0 mg, 0.258 mmol) was added, the reaction vial was sealed with cap and electrical tape immediately. After the reaction mixture was heated overnight at 60° C., the formation of the product was confirmed by thin layer chromatography. The volatiles were removed by the gentle flow of N2 gas, and then the resulting solid was reconstituted in H₂O (2 mL). The product was extracted from the aqueous phase with diethyl ether (2×3 mL), dried with Na₂SO₄ and filtered into a 20-mL vial. The mixture was dried under vacuum to afford a yellow waxy solid (29.1 mg, 57%). ¹H NMR (500 MHz, CDCl₃): δ 8.23 (d, J=10.0 Hz, 1H), 8.15 (m, 4H), 7.98 (m, 4H), 4.99 (s, 2H). ¹⁵N {¹H} NMR (60 MHz, CDCl₃, ¹⁵N-enriched sample): δ 78.5.

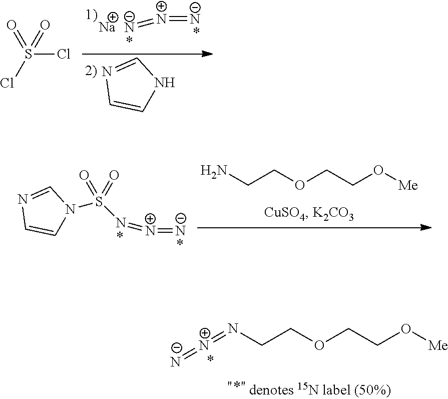

Synthesis of ¹⁵N-enriched alkylazide at the internal nitrogen: The synthetic procedure was adopted from previous reports using ¹⁵N-enriched sodium azide (#609374; MilliporeSigma, Burlington, Mass., United States of America).^{127,128} To a 4-mL vial, ¹⁵N-enriched sodium azide (69.9 mg, 1.08 mmol) was added. Dry MeCN (1 mL) was added to the vial in air, and the suspension was cooled down in an ice bath for 2 min. Sulfuryl chloride (87.4 μL, 1.08 mmol) was added to the vial in air with a mechanical pipette, and the mixture was stirred at rt overnight. The reaction mixture was cooled in an ice bath for 2 min, and imidazole (147.9 mg, 2.16 mmol) was added in two portions. After the reaction mixture was stirred at rt for 3 h, the formation of the imidazolyl sulfonylazide product was confirmed by silica gel thin layer chromatography using 1:1 EtOAc/hexane as an eluent (R_{f} ˜0.4). EtOAc (3 mL) was added to the reaction mixture, and the organic layer was washed with water (1 mL) and saturated NaHCO₃ solution (1 mL), dried over Na₂SO₄, filtered, and dried by the gentle flow of nitrogen gas. Shortly after the removal of the solvents by the nitrogen gas flow, the sulfonyl azide intermediate was dissolved in methanol (1.5 mL) for the azide transfer reaction to prevent its decomposition. In a 4-mL vial, 2-(2-methoxyethoxy)ethanamine (103.0 mg, 0.864 mmol) and K₂CO₃ (113.0 mg, 0.818 mmol) were suspended in methanol (1 mL), and CuSO₄.5H₂O (2.6 mg, 0.010 mmol) was added. To the blue suspension, the solution of the sulfonyl azide in methanol was added in two portions (i.e. 2×0.75 mL). After the mixture was stirred at rt overnight, the resulting light gray suspension was concentrated to ˜0.25 mL by the gentle flow of nitrogen gas, and the formation of the alkylazide product as well as the disappearance of the amine starting material were confirmed by silica gel thin layer chromatography (4:1 EtOAc/hexane) using anisaldehyde and ninhydrin stain, respectively. Water (6 mL) and HCl aqueous solution (1 mL, 6 M stock) was added, and the product was extracted with EtOAc (3×5 mL). The combined organic layer was dried over Na₂SO₄, filtered, and dried under vacuum to afford the crude product of the alkylazide as light yellow oil (84.0 mg, 67%). The crude material was used without further purification for the phosphine-mediated reaction. ¹H NMR (500 MHz, CDCl₃): δ. 3.66 (m, 4H), 3.56 (m, 2H), 3.39 (m, 5H). ¹⁵N {¹H} NMR (60 MHz, CDCl₃, ¹⁵N-enriched sample): δ 248.2.

### Example 36

### Discussion of Examples 26-35

Through various characterization methods, it has been confirmed that the product of the phosphine-mediated azide-anine coupling reaction is a urea group. Although a tetrazene structure with four consecutive nitrogen atoms aligned linearly was originally proposed as the structure of the transformation based mainly on mass spectrometry and ¹⁵N NMR experiment, it was found that one of the observed signals in ¹⁵N NMR around 300 ppm (N═N in the originally proposed tetrazene structure, corresponding to the terminal azide depicted in FIG. 15A) was an artifact and additional data suggested a partial loss of the ¹⁵N-labeled nitrogen after the reaction. To fully understand the product structure, ¹⁵N isotope was incorporated into each of the four possible nitrogen atoms of the amine and azide starting materials, and the introduction of the isotope was found only for the two positions of the product (see FIG. 15A), confirmed by NMR and mass spectrometry. Such partial loss of the ¹⁵N isotopes from the azide reagents was not observed when the same azide reagents were subjected to the well-known azide-alkyne cycloaddition, which validates the presence of the ¹⁵N isotopes in the azide compounds and their loss specifically by the phosphine-mediated chemistry. Using amino-pyrene and azido-pyrene starting materials, we were able to obtain crystal structure of the reaction product, which suggests the branched linkage corresponding to the urea group (see FIG. 15B), as the bond lengths and angles of the crystal structure represents a typical urea structure.¹²⁹ Liquid chromatography-mass spectrometry (LC-MS) analysis of a product of a reaction under ¹³C-labeled carbon dioxide showed a 1-Da mass shift, compared to the reaction under air and with ¹²CO₂. See FIG. 15C. Introduction of the ¹³C isotope was also observed for the reaction with ¹³C-labeled potassium carbonate used as an additive too whereas such mass shift did not occur with ¹⁸O-labeled water, altogether confirming that the C═O fragment originates from carbon dioxide. The ¹³C isotope incorporation was observed in the product of not only the model substrate pyrene, but also for the amine-containing peptide, DNA, and carbohydrate substrate. In addition, ¹³C NMR spectra of the products exhibited signature peak around 158 ppm corresponding to the urea carbonyl carbon with and without the ¹⁵N-label-based peak splitting patterns. See FIG. 15D. Furthermore, the C═O stretch of the urea group was found in infrared spectroscopy.¹³⁰

Without being bound to any one theory, the phosphine-mediated urea forming chemistry is believed to proceed via the iminophosphorane-carbon dioxide reaction. The loss of two nitrogen atoms observed in LC-MS and NMR experiments suggests the generation of iminophosphorane intermediate,²⁹ which is known to react with CO₂ generating isocyanate intermediate.¹³¹ Indeed, a recent report demonstrated that the urea formation through iminophosphorane intermediate is exceptionally efficient even with small concentration of carbon dioxide.¹³² and this attribute possibly provided the application of the presently disclosed ionic liquid-based bioconjugation solely with CO₂ at the atmospheric level. Due to their hydrolytic instability,¹³³ in situ generation of isocyanates has provided a convenient and practically useful methodology for bioconjugation processes,¹³⁴ which could function as an amine-targeting labeling approach.¹³⁵

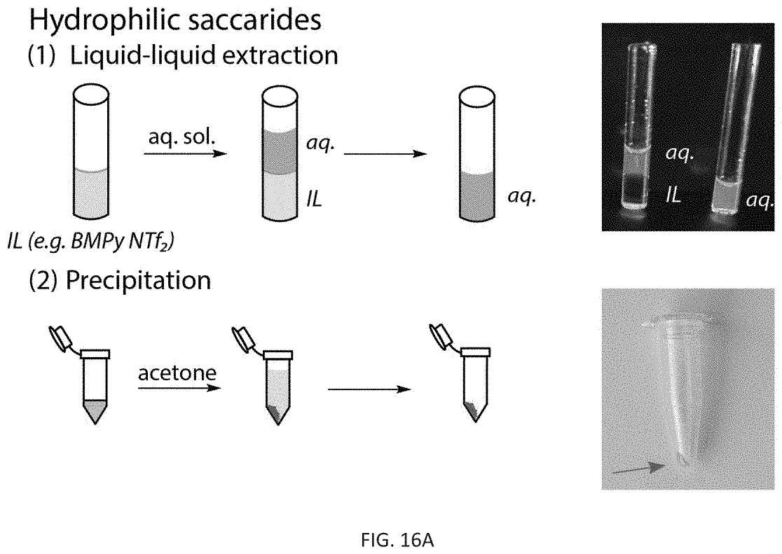

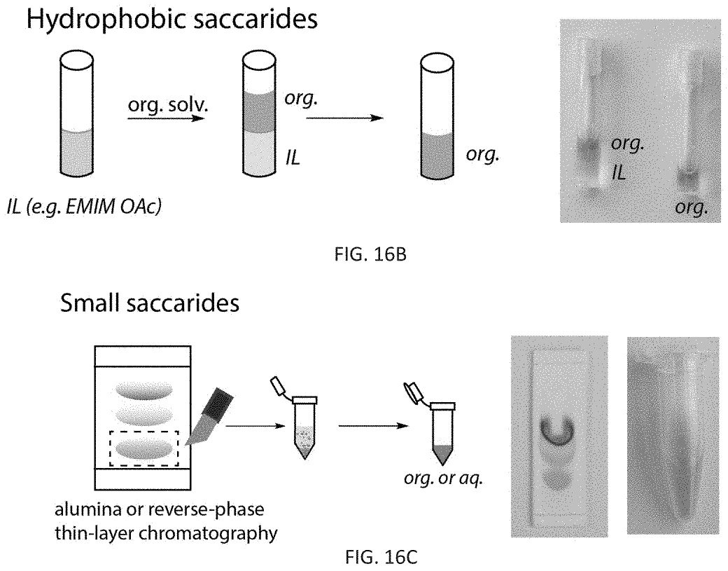

To facilitate the ionic liquid-based bioconjugation of various saccharides with the diverse structure and solubility, multiple methods were developed to separate the modified saccharides from the ionic liquid-containing reaction mixture. See FIGS. 16A-16C. While a collective set of protocols for the compound extraction using ionic liquid has been available.¹³⁶ difficulty separating the target saccharides from ionic liquids and excess chemical labeling reagents was observed in microliter-scale reaction screening processes. It is believed that the majority of the previous efforts for the ionic liquid-based purification have been focused mainly on a large, industrial scale rather than small-scale reaction screening process, let alone a bioconjugation process.¹³⁶ Precipitation of target biomolecules with organic solvent (e.g. acetone) was used as in the protein and DNA modification studies described above to remove ionic liquid and excess chemical reagents. Although this precipitation approach was applicable to some relatively large saccharides (see FIG. 16A), significant loss of the samples was observed for many other carbohydrate derivatives.

To this end, a microliter-scale liquid-liquid extraction protocol was established for the bioconjugation screening processes by using different combinations of the ionic liquid and phosphine reagent. See FIGS. 16A and 16B. For instance, hydrophilic saccharides were successfully extracted through a bilayer system of water and a water-immiscible ionic liquid, such as bis(trifluoromethanesulfonyl)imide (NTf₂)-based ionic liquid, and the separated aqueous solution containing the desired saccharides can be further washed by organic solvent such as ethyl acetate. While water-immiscible (or relatively more hydrophobic) ionic liquids can be less convenient as a reaction medium for such hydrophilic carbohydrate due to their limited dissolution properties, it was discovered that this extraction system can be applied for a reaction in water-miscible ionic liquid (e.g. butylmethylpyrrolidinium trifluoromethanesulfonate, BMPy OTf) by addition of a water-immiscible ionic liquid after the reaction. For example, a product of a reaction performed in water-miscible BMPy OTf can be extracted with water or buffer by the addition of water-immiscible BMPy NTf₂, as the water-immiscible ionic liquid induces the phase separation from water even in the presence of the water-miscible ionic liquid. This process can be translated for hydrophobic saccharide targets by using ionic liquids immiscible with organic solvents during the reaction, and the desired hydrophobic compound can be extracted with an organic solvent, such as ethyl acetate. See FIG. 16B. In addition to the extraction protocols, a screening process for small saccharides using normal-phase (alumina) or reverse-phase (C18) thin-layer chromatography (TLC) was developed, as well. See FIG. 16C. These established protocols are not only conducive for the presently disclosed phosphine-mediated chemistry, but also for future development of ionic liquid-based reaction screening processes for biomolecules including carbohydrates, peptides/proteins, and nucleotides.

With multiple purification methods in hand, the selectivity of the phosphine-mediated reaction toward therapeutically important saccharides with different types of amine groups was examined. See FIG. 17A. To begin with, a reaction survey of different types of amine groups was conducted on a simple small molecule (anthracene), including primary, secondary, tertiary alkylamine as well as primary arylamine. See FIG. 17B. The amine compounds were incubated with azide 2 and triphenylphosphine, and the reaction mixture was subjected to the thin-layer chromatography clean-up process, as well as to liquid-chromatography (LC) analysis. The LC analysis showed that product formation was observed for primary and secondary alkylamines under the phosphine-mediated reaction, whereas the tertiary alkylamine and primary arylamine did not display meaningful reactivity, consistent with the presently disclosed peptide and DNA modification. Having understood the reactivity toward not only primary but also secondary alkylamine groups, modification of primary and secondary amine-containing small molecule carbohydrates was studied. See FIGS. 17C-17E. Doxorubicin is an anti-tumor agent containing one primary alkylamine group, and its chemical modification has been part of emerging therapeutic strategies.^{137,138} According to the presently disclosed subject matter, it was found that the reaction of doxorubicin with alkylazide reagent produced a single modification product, and the phosphine-dependent nature of the reaction was also demonstrated. See FIGS. 17C and 17D. To verify the amine dependence of the modification, valrubicin bearing trifluoroacetyl group-capped amine was used. Despite the structural similarity of valrubicin to doxorubicin, the reaction of valrubicin resulted in the observation of only intact starting material. To better understand the generality of the presently disclosed azide-amine bioconjugation chemistry on other therapeutically important saccharides, the antibiotic vancomycin and its derivatives were tested, and the modification efficiency was assessed by LC-MS. See FIG. 17E. The parent vancomycin contains one primary and one secondary alkylamine and, consistent with the aforementioned selectivity survey, the vancomycin reaction resulted in up to 2 modifications. Other vancomycin derivatives exhibited the similar trends; fluorescein isothiocyanate (FITC)-modified vancomycin containing only one secondary amine group provided one modification, and oritavancin with an additional secondary amine compared to parent vancomycin gave up to 3 modifications. Thus, the presently disclosed phosphine-mediated chemistry can be applicable to different types of therapeutic carbohydrates with primary and secondary alkylamine groups.

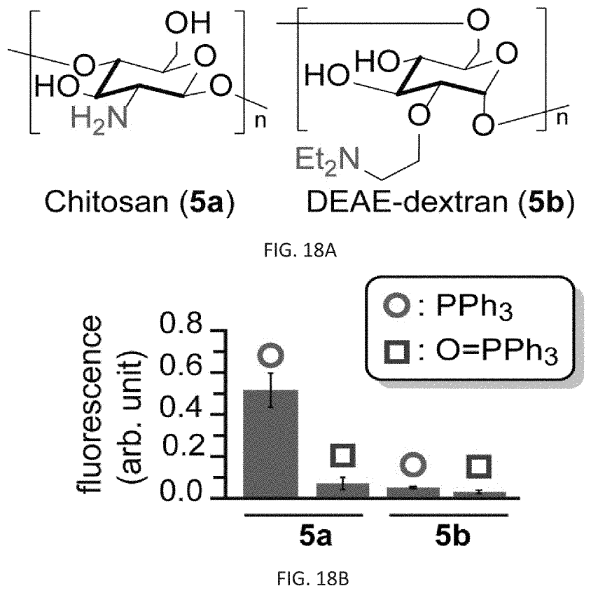

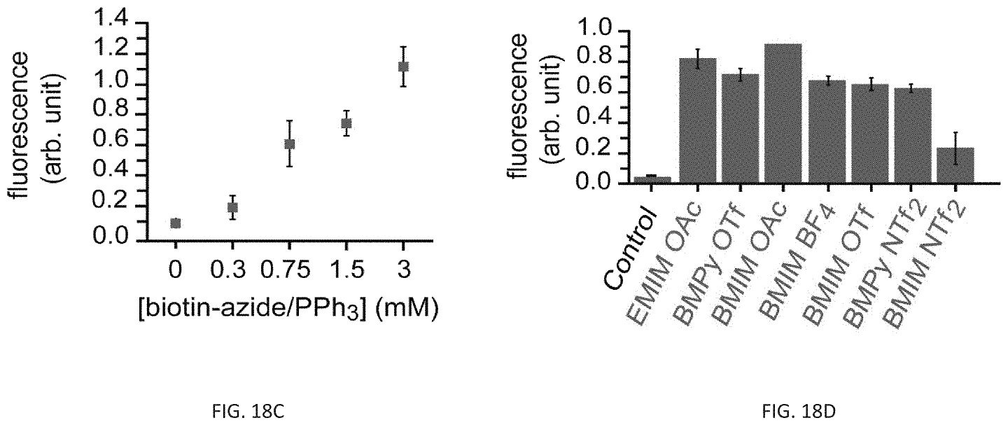

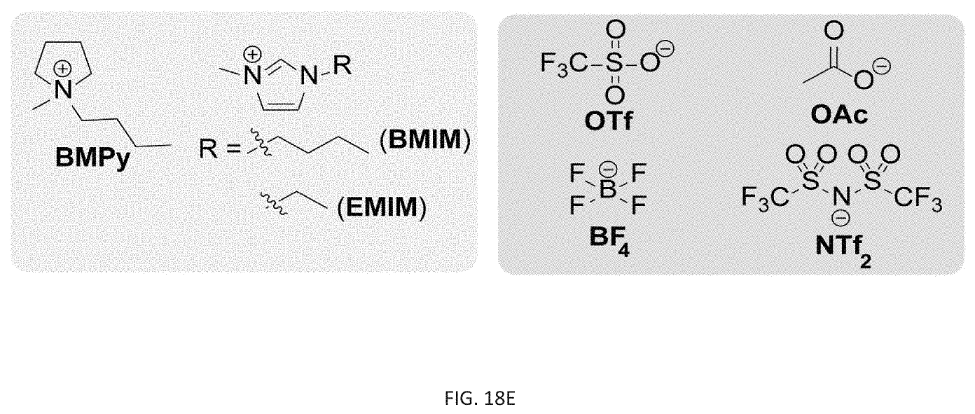

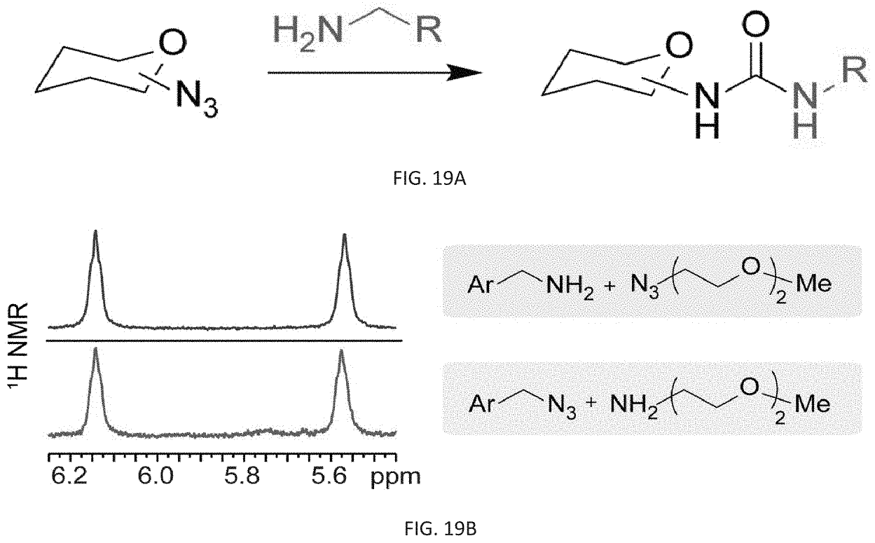

Having confirmed the successful bioconjugation of small molecule carbohydrate derivatives, we have turned our attention to amine-containing polysaccharide substrates. See FIGS. 18A-18E. Chitosan is an aminopolysaccharide that can cause nanoparticle formation to encapsulate and deliver desired molecules for a variety of applications, and bioconjugation of chitosan is a powerful tool to append additional capability to the nanoparticle carrier.^{139,140} Thus, the reactivity of chitosan toward the phosphine-mediated chemistry was investigated. Alkylazide-functionalized biotin was used for this study, as fluorescence signals from an anti-biotin blot with streptavidin-fluorophore conjugate would be proportional to the labeling efficiency. Similar blot-based analysis of polysaccharides have been reported previously.¹⁴¹ As a comparison, diethylaminoethyl (DEAE)-dextran bearing tertiary alkylamine groups was also subjected to the reaction conditions. Strong signal was observed exclusively from the chitosan reaction with the triphenylphosphine reagent but not from the reactions of DEAE-dextran with PPh₃ nor from chitosan with O═PPh₃ (see FIGS. 18A and 18B), in harmony with the reactivity survey in FIG. 17B. The modification efficiency proved dependent on concentration of azide reagents (see FIG. 18C), and this concentration dependence can provide for incorporation of a desired number of functionalities onto the polysaccharide for suitable applications. Consistent with the DNA modification described herein, a variety of ionic liquids are amenable for this chemical transformation (see FIGS. 18D and 18E), except for the reaction in BMIM NTf₂ which showed low labeling efficiency, which, without being bound to any one theory is believed due to the poor solubility of chitosan in this ionic liquid. The robustness of the phosphine-mediated modification reaction in different ionic liquids reinforces the value of the presently disclosed bioconjugation approach.

The amine-azide coupling reaction can be translated for labeling of azide-containing saccharides. See FIG. 19A. There is a large availability of azide-tagged carbohydrates in market and literature due largely to the rise of biorthogonal chemistry.¹⁴² As the modification of amine-containing saccharides has been successful with excess azide and phosphine reagents, it was hypothesized that azidocarbohydrates could be chemically labeled with excess amine reagents in the same fashion. To validate the hypothesis, an LC-MS and NMR analysis was conducted on small molecule substrate, azide-pyrene with excess amine reagents. The purified reaction product of this reaction showed the formation of the same product as the previously reported reaction of amino-pyrene and azide reagent in ¹H NMR displaying the distinct peaks for the newly formed NH groups. See FIG. 19B. Confirming that labeling of azide-containing substrates with excess amine reagents is possible, modification of an oligosaccharide containing an alkylazide handle was tested. A nonasaccharide containing a single alkylazide group was incubated with excess primary, secondary, and tertiary benzylamine reagents, and the reaction was analyzed by matrix-assisted laser desorption/ionization (MALDI)-MS. See FIG. 19C. The results were consistent with the selectivity survey depicted in FIG. 17B, as appreciable reactivity was observed from primary and secondary alkylamine conditions but not tertiary alkylamines, and triphenylphosphine oxide negative control showed the absence of the reactivity.

Encouraged by the promising results of the small azide-containing saccharides, modification of polysaccharides containing alkylazide groups with different types of functionalized amine reagents was tested. See FIGS. 19D and 19E. Hyaluronic acid is a polysaccharides composed of glucuronic acid and acetylmannosamine units and has been applied for various medical purposes that include the use of chemically functionalized hyaluronate.^{143,144} Along with this application, labeling of hyaluronic acid with bioorthogonal handles (e.g. azide, terminal alkyne, and cyclooctyne) has been increasingly studied as well.¹⁴⁵⁻¹⁴⁷ The reaction of the azide-labeled hyaluronic acid in ionic liquid proceeded smoothly with excess fluorophore-amine, compared to the parent hyaluronic acid, as analyzed by fluorescence imaging on a blot membrane. See FIG. 19D. In addition, bioorthogonal handles such as terminal alkyne and trans-cyclooctene can be incorporated through this method, and the secondary modification with corresponding bioorthogonal chemistry proved feasible using azide (copper-catalyzed azide-alkyne cycloaddition)¹⁴⁸ and tetrazine (inverse electron-demand Diels-Alder reaction),¹⁴⁹ respectively. See FIG. 19E.

The strategy to label azide-containing carbohydrates was further tested in a complex cellular system. See FIGS. 19F and 19G. Periodate oxidation and a subsequent hydrazone forming reaction were employed to introduce azide groups into saccharide groups of glycoproteins, as this approach has been utilized for various biological samples including mammalian cells and zebra fish.^{150,151} To the lysate of human embryo kidney (HEK) 293T cells, sodium periodate solution was added, and after removal of the oxidizing reagent, the oxidized cell lysate was tagged with hydrazide reagent containing alkylazide group. The introduction of the azide group to the lysate was confirmed by the well-established copper-catalyzed azide-alkyne cycloaddition reaction. Then, the azide-tagged cell lysate was treated with alkylamine-containing biotin (see FIG. 19F) and triphenylphosphine in ionic liquid, and the reaction was analyzed by anti-biotin western blot. See FIG. 19G. Fluorescence signals were observed strongly from the oxidized/azide-tagged sample from the blot membrane while the negative control condition without the oxidation step showed insignificant signals. Thus, the presently disclosed ionic liquid-based approach proved translatable into even a complex mixture of biomolecules such as mammalian cell lysate.

Accordingly, the phosphine-mediated chemistry in ionic liquid described herein provided selective, efficient labeling of various types of amine- and azide-containing saccharides through the in situ generation of a reactive electrophile, offering a facile and convenient method for the carbohydrate bioconjugation research. The capability to label azide-containing saccharides is of particular note, as a number of site-specific incorporations of alkylazide groups has been developed, and, thus, the amine-azide coupling method could potentially function as a site-specific labeling technology as well. Various carbohydrates are amenable for this chemical transformation ranging from monosaccharides to polysaccharides with intricate structures, proving its versatility. Applicability to therapeutic carbohydrates, such as antibiotics (vancomycin) and anti-tumor agents (doxorubicin), expands the application of the presently disclosed bioconjugation of saccharides to medicinal chemistry using nontraditional media. In addition to the utility of the bioconjugation reactions, the present disclosure also provides protocols for screening of bioconjugation reactions in ionic liquid through multiple reaction cleanup methods. Moreover, it is believed that the present disclosure represents the first ionic liquid-based bioconjugation of cellular samples/mammalian cell lysate, which could facilitate the future applications of the bioconjugation in nonaqueous medium as a bioorthogonal chemistry-like strategy.

### REFERENCES

All references listed herein including but not limited to all patents, patent applications and publications thereof, scientific journal articles, and database entries are incorporated herein by reference in their entireties to the extent that they supplement, explain, provide a background for, or teach methodology, techniques, and/or compositions employed herein.
(1) Walsh et al., Chem. Soc. Rev 2021, 50, 1305.
(2) Spradlin et al., Acc. Chem. Res. 2021, 54, 1801-1813.
(3) Niphakis et al., Annu. Rev. Biochem. 2014, 83, 341.
(4) Liang et al., Proc. Natl. Acad Sci. U.S.A. 2019, 116, 15453-15462.
(5) Alcock et al., Chem. Soc. Rev. 2018, 47, 231-268.
(6) Kang et al., J. Am. Chem. Soc. 2021, 143, 5616-5621.
(7) Ito et al., Angew. Chem. Int. Ed 2018, 130, 15928-15932.
(8) Iwashita et al., Proc. Natl. Acad Sci. U.S.A. 2021, 118, e2018513118.
(9) Messina et al., Prog. Polym. Sci. 2020, 100, 101186.
(10) Row et al., Acc. Chem. Res. 2018, 51, 1073-1081.
(11) MacDonald et al., Nat. Chem. Biol. 2015, 11, 326-331.
(12) Tamura et al., J. Am. Chem. Soc. 2019, 141, 2782-2799.
(13) Hoyt et al., Nat. Rev. Chem. 2019, 3, 147.
(14) Purushottam et al., Nat. Commun. 2019, 10, 2539.
(15) Adusumalli et al., Angew. Chem. Int. Ed. 2020, 59, 10332-10336.
(16) Sletten et al., Angew. Chem. Int. Ed. 2009, 48, 6974-6998.
(17) Zhang et al., Angew. Chem. Int. Ed 2019, 58, 4810-4839.
(18) Ohata et al., Angew. Chem. Int. Ed. 2019, 58, 6176-6199.
(19) Bommarius et al., J. Am. Chem. Soc. 1995, 117, 4515-4523.
(20) Cortes-Clerget et al., Nat Commun 2019, 10, 2169.
(21) Lei et al., Chem. Rev. 2017, 117, 6633-6635.
(22) Egorova et al., Chem. Rev. 2017, 117, 7132-7189.
(23) Parvulescu and Hardacre, Chem. Rev. 2007, 107, 2615-2665.
(24) Gomes et al., Chem. Soc. Rev. 2019, 48, 4317-4335.
(25) Shukla and Mikkola, Front. Chem. 2020, 8, 1219.
(26) Schindl et al., Front Chem 2019, 7, 347.
(27) Brogan and Hallett. J Am. Chem. Soc. 2016, 138.4494-4501.
(28) Gu et al, Macromolecules 2014, 47, 6740-6747.
(29) Bednarek et al., Chem. Rev 2020, 120, 4301.
(30) Tetrazene NH proton was observed at 5.88 ppm as a broad peak in a previous report (see reference 31).
(31) Haines et al., J. Org. Chem. 1982, 47, 474-482.
(32) Klahn et al., Angew. Chem. Int. Ed 2014, 53, 7913-7917.
(33) Dhenain et al., Chem. Eur. J. 2017, 23, 9897-9907.
(34) Sebate and Delalu. Cent. Eur. J Energ. Mat. 2014, 11, 515.
(35) LaBonte et al., Nature Reviews Drug Discovery 2003, 2, 345-346.
(36) Eymann et al., Angew. Chem. Int. Ed. 2021, 60, 1578-1582.
(37) Wiberg et al., Chem. Ber. 1980, 113, 2916-2927.
(38) Chan et al., Anal. Chem. 2017, 89, 9976-9983.
(39) Wang and Xu, Signal Transduct. Target. Ther. 2019, 4, 1-22.
(40) Beck et al., Nat Rev Drug Discov 2017, 16, 315-337.
(41) Shaffer, Nat. Biotechnol. 2021, 39, 128-130.
(42) Myers and Raines, Angew. Chem. Int. Ed. 2009, 48, 2359-2363.
(43) Chou and Raines, J. Am. Chem. Soc. 2013, 135, 14936-14939.
(44) Meguro et al., Org. Lett. 2018, 20, 4126-4130.
(45) Tannoux, et al., J. Org. Chem. 2021, 86, 3017-3023.
(46) Taylor and Lohani, Org. Lett. 2016, 18, 4412-4415.
(47) Desamparados Velasco et al., Tetrahedron 2000, 56, 4079-4084.
(48) Reaction of PPh3 and azide 2 in the presence of BMPy OTf produced a unique phosphorous species in 31P NMR, compared to the reaction without BMPy OTf. Relatively low dielectric constant of ionic liquids may be contributing to this observation (see references 49 and 50).
(49) Rybinska-Fryca et al., J. Mol. Liq. 2018, 260, 574A.
(50) Lin et al., J. Am. Chem. Soc. 2005, 127, 2686-2695.
(51) Kennedy et al., Nature 2021, 593, 223.
(52) Wang et al., Nat. Chem. 2021, 13, 378.
(53) Das, Chem. Rev. 2011, 111, 4405-4417.
(54) Wu and Devaraj, Acc. Chem. Res. 2018, 51, 1249-1259.
(55) Lee et al., J Am. Chem. Soc. 1987, 109, 5050-5051.
(56) Lenstra et al., J Org. Chem. 2019, 84, 6536-6545.
(57) Wu, et al., Angewandte Chemie International Edition 2004, 43, 3928-3932.
(58) Zhao et al., Bioconjugate Chem. 2008, 19, 849-859.
(59) Gu et al., The Journal of Antibiotics 2011, 64, 111-116.
(60) Moon, et al., J. Med. Chem. 2008, 51, 6916-6926.
(61) Sawant et al., Org. Lett. 2018; 20(15), 4397-4400.
(62) Moutiers et al., J. Chem. Soc., Perkin Trans. 2, 1999, 1287-1288.
(63) Borodkin et al., Russian Journal of Physical Chemistry A., 2019, 93(11), 2187-2191.
(64) Zheng et al., Chem Soc Rev, 2015, 44, 3036-3055.
(65) Monroy-Contreras and Vaca, J Nucleic Acids, 2011, 2011, 741723.
(66) Smith and Osborn, FEMS Microbiol Ecol, 2009, 67, 6-20.
(67) Ting et al., J. Am. Chem. Soc., 2004, 126, 12720-12721.
(68) Wang et al., ACS Chem. Biol., 2016, 11, 444-451.
(69) Boersma et al., Chem. Soc. Rev., 2010, 39, 2083-2092.
(70) Madsen and Gothelf, Chem. Rev., 2019, 119, 63844458.
(71) Kulkami et al., Nat. Nanotechnol., 2021, 16, 630-(43.
(72) Young et al., J. Am. Chem. Soc., 2010, 132, 6183-6193.
(73) Ostergaard et al., Nucleic Acids Research, 2020, 48, 1691-1700.
(74) Ma et al., PLoS One, 2015, 10, e0124924.
(75) Struntz and Harki, ACS Chem. Biol., 2016, 11, 1631-1638.
(76) Yu et al., Mol. Pharmaceutics, 2017, 14, 2815-2823.
(77) Meng et al., BMC Biotechnology, 2011, 11, 88.
(78) Hughes and Ellington, Cold Spring Harb Perspect Biol, 2017, 9, a023812.
(79) Xu et al., J Am. Chem. Soc., 2020, 142, 5785-5792.
(80) Tishinov et al., Chem. Sci., 2013, 4, 4401-4406.
(81) Geigle et al., Chem. Sci., 2016, 8, 499-506.
(82) Gillingham and Shahid, Curr Opin Chem Biol, 2015, 25, 110-114.
(83) Li and Moellering, ChemBioChem, 2019, 20, 1599-1605.
(84) Li et al., Proc. Natl. Acad. Sci. U.S.A., 2019, 116, 21493-21500.
(85) Robinson et al., J. Am. Chem. Soc., 2016, 138, 10722-10725.
(86) Jang et al., Nucleic Acids Research, 2019, 47, e102-e102.
(87) Liu et al., Nat Commun. 2020, 11, 4857.
(88) Hu et al., FEBS Letters, 2019, 593, 1113-1121.
(89) Gao et al., Chem. Sci., 2019, 10, 6035-6071.
(90) Wu et al., J. Mater. Chem., 2011, 21, 13611-13620.
(91) Sun et al., J. Mater. Chem., 2010, 20, 5605-5607.
(92) Nantalaksakul et al., J. Am. Chem. Soc., 2009, 131, 2727-2738.
(93) Kuntip et al., Biopolymers, 2021, 112, e23421.
(94) Stewart et al., Organometallics, 2011, 30, 5338-5343.
(95) Kiernan, Biotechnic & Histochemistry, 2018, 93, 133-148.
(96) Cao et al., Angew. Chem. Int. Ed., 2009, 48, 6494-6498.
(97) Wan et al., Cancer Res, 2018, 78, 798-808.
(98) Sekkai et al., Antisense and Nucleic Acid Drug Development, 2002, 12, 265-274.
(99) Ohmann et al. Nucleic Acids Res., 2019, 47, 11441-11451.
(100) Jones et al., J. Am. Chem. Soc., 2021, 143, 8305-8313.
(101) Iqbal and Iqbal, Molecular Biology International, 2014, 2014, e852748.
(102) Mahlknecht et al. Proc. Natl. Acad. Sci. U.S.A., 2013, 110, 8170-8175.
(103) Fantoni et al., Chem. Rev., 2021, 121, 7122-7154.
(104) Wang et al., Angew. Chem. Int. Ed., 2018, 57, 15787-15791.
(105) MacFarlane et al., Faraday Discuss., 2017, 206, 9-28.
(106) Mettu et al., J. Biomed. Sci. 2020, 27, 9.
(107) Klyosov, in Glycobiol. Drug Des., American Chemical Society, 2012, pp. 3-22.
(108) Umstatter et al., Angew. Chem. Int. Ed. 2020, 59, 8823-8827.
(109) Cyphert et al., Antibiot. Basel Switz. 2017, 6, E11.
(110) Chtchigrovsky et al., Angew. Chem. Int. Ed. 2009, 48, 5916-5920.
(111) Szabo et al., Polymers 2020, 12, 919.
(112) Lizundia et al., in Multifunct. Polym. Nanocomposites Based Cellul. Reinf. (Eds.: D. Puglia, E. Fortunati, J. M. Kenny), William Andrew Publishing, 2016, pp. 61-113.
(113) Heinze et al., in Polysacch. II (Ed.: D. Klemm), Springer, Berlin, Heidelberg, 2006, pp. 199-291.
(114) Prestwich et al., J. Controlled Release 1998, 53, 93-103.
(115) Zhou et al., Bioconjug. Chem. 2014, 25, 510-520.
(116) Zeglis et al., Bioconjug. Chem. 2013, 24, 1057-1067.
(117) Moon et al., JACS Au 2022, 2, 74-83.
(118) Zhu et al., Angew. Chem. Int. Ed 2016, 55, 9301-9305.
(119) Debets et al., Proc. Natl. Acad. Sci. 2020, 117, 25293-25301.
(120) Chang et al., Proc. Natl. Acad Sci. 2010, 107, 1821-1826.
(121) Boons, in Carbohydr. Chem., 2010, pp. 152-167.
(122) Sletten and Bertozzi, Acc. Chem. Res. 2011, 44, 666-676.
(123) Shiramizu and Toste, Angew. Chem. Int. Ed Engl. 2013, 52, 12905-12909.
(124) Li et al., Angew. Chem. Int. Ed Engl. 2014, 53, 7179-7182.
(125) Tsai et al., ACS Omega 2018, 3, 4423-4432.
(126) Park et al., J. Org. Chem. 2008, 73, 8212-8218.
(127) Goddard-Borger and Stick, Org. Lett. 2007, 9, 3797-3800.
(128) Pandiakumar et al., Tetrahedron Letters 2014, 55, 2917-2920.
(129) Wang et al., Tetrahedron 2011, 67, 1530-1535.
(130) Wu et al., Synth. Commun. 2016, 46, 1364-1369.
(131) Del Vecchio et al., Angew. Chem. Int. Ed 2018, 57, 9744-9748.
(132) Babin et al., Chem. Commun. 2021, 57, 6680-6683.
(133) Brown et al., Environ. Health Perspect. 1987, 72, 5-11.
(134) Vinogradov et al., Org. Lett. 2016, 18, 1222-1225.
(135) Sato et al., ACS Chem. Biol. 2015, 10, 2633-2640.
(136) Ventura et al., Chem. Rev. 2017, 117, 6984-7052.
(137) Wang et al., J. Am. Chem. Soc. 2019, 141, 17133-17141.
(138) Li and Chen, Nat. Chem. Biol. 2016, 12, 129-137.
(139) Naskar et al., J. Drug Target. 2019, 27, 379-393.
(140) Smith et al., Oncoimmunology 2017, 6, e1259050.
(141) Van Dyken et al., J., Immunol. Baltim. Md 1950 2011, 187, 2261-2267.
(142) Patterson et al., ACS Chem. Biol. 2014, 9, 592-605.
(143) Schante et al., Carbohydr. Polym. 2011, 85, 469-489.
(144) Kogan et al., Biolechnol. Lett. 2007, 29, 17-25.
(145) Hu et al., Acta Biomater. 2011, 7, 1618-1626.
(146) Han, et al., Polym. Chem. 2017, 9, 20-27.
(147) Piluso et al., Int. J. Artif. Organs 2011, 34, 192-197.
(148) Ohata et al., Mol. Biosyst. 2015, 11, 2846-2849.
(149) Devaraj et al., Angew. Chem. Int. Ed. 2009, 48, 7013-7016.
(150) Key et al., Bioconjug. Chem. 2012, 23, 363-371.
(151) Baskin et al., Proc. Natl. Acad. Sci. 2010, 107, 10360-10365.

It will be understood that various details of the presently disclosed subject matter may be changed without departing from the scope of the presently disclosed subject matter. Furthermore, the foregoing description is for the purpose of illustration only, and not for the purpose of limitation.

What is claimed is:

1. A method of performing a chemoselective bioconjugation reaction, the method comprising contacting a biomolecule substrate with a functionalized molecule, wherein said biomolecule substrate comprises one of the group consisting of a peptide, a protein, and a nucleic acid, and wherein said functionalized molecule comprises at least one chemical functional group that can form a bond with a chemical functional group present in said biomolecule substrate, and wherein the contacting is performed in a reaction mixture comprising a solvent or solvent mixture comprising, consisting essentially of, or consisting of, an ionic liquid, thereby forming a bioconjugate product.

2. The method of claim 1, wherein the biomolecule substrate comprises one of the group consisting of an enzyme, an antigenic protein, a chemokine, a cytokine, a cellular receptor, a cellular receptor ligand, an aptamer, and an antibody or active fragment thereof.

3. The method of claim 1, wherein the biomolecule substrate comprises one or more aminoalkyl moiety; wherein the functionalized molecule is an azide-containing compound; wherein the reaction mixture further comprises a triarylphosphine; and wherein the bioconjugation product comprises an urea linkage.

4. The method of claim 3, wherein the aminoalkyl moiety comprises an amino group of a terminal amino acid residue in a protein or peptide or an amino group of a lysine residue side chain in a peptide or protein.

5. The method of claim 3, wherein the azide-containing compound comprises an azide-containing derivative of one of the group consisting of a small molecule therapeutic agent; a nucleic acid; a lipid; a carbohydrate: a polymer; and a detectable label.

6. The method of claim 3, wherein the ionic liquid comprises one or more of the group consisting of 1-butyl-1-methylpyrrolidinium trifluoromethanesulfonate (BMPy OTf), 1-butyl-1-methylpyrrolidinium bis(trifluoromethanesulfonyl)imide (BMPy NTf₂), 1-ethyl-3-methylimidazolium acetate (EMIM OAc), 1-butyl-3-methylimidazolium acetate (BMIM OAc), 1-butyl-3-methylimidazolium trifluoromethanesulfonate (BMIM OTf), 1-butyl-3-methylimidazolium bis(trifluoromethanesulfonyl)imide (BMIM NTf₂), 1-butyl-3-methylimidazolium tetrafluoroborate (BMIM BF₄), and tributylethylphosphonium diethylphosphate (TBEP).

7. The method of claim 3, wherein the contacting is performed at a temperature of about 20 degrees Celsius (° C.) to about 70° C.

8. The method of claim 3, wherein the contacting is performed for about 30 minutes to about 72 hours.

9. The method of claim 3, wherein the biomolecule substrate is present in the reaction mixture at a concentration of about 0.025 millimolar (mM) to about 0.4 mM; wherein the azide-containing compound is present at a concentration of about 3 mM to about 20 mM, and wherein the triarylphosphine is present at a concentration of about 3 mM to about 7.5 mM.

10. The method of claim 3, wherein the reaction mixture further comprises a bicarbonate buffer, a borate buffer, or an acetate buffer.

11. The method of claim 3, wherein the reaction mixture comprises no more than 6% by volume water.

12. The method of claim 1, wherein the biomolecule substrate comprises a protein comprising one or more carboxylic acid group: the functionalized molecule comprises an amino group; the reaction mixture further comprises a diboron compound; and the bioconjugation product comprises an amide linkage.

13. The method of claim 1, wherein the biomolecule substrate comprises a protein comprising one or more aminoalkyl group; the functionalized molecule comprises a triarylphosphonium aldehyde; and the bioconjugation product comprises an enamine linkage.

14. A method of performing a chemoselective bioconjugation reaction, the method comprising contacting a first molecule and a second molecule in a reaction mixture comprising a triarylphosphine and a solvent or solvent mixture, wherein said first molecule comprises an aminoalkyl group, wherein said second molecule comprises an azide group, and wherein the solvent or solvent mixture comprises, consists essentially of, or consists of an ionic liquid, thereby forming a bioconjugate product comprising a urea linkage, and wherein at least one of said first molecule and said second molecule comprises a biomolecule or a derivative thereof, wherein said biomolecule or derivative thereof comprises a biomolecule or derivative selected from the group consisting of a protein, a peptide, a nucleic acid, a carbohydrate, and derivatives thereof.

15. The method of claim 14, wherein the solvent or solvent mixture comprises 6% by volume water or less.

16. The method of claim 14, wherein the first molecule is present in the reaction mixture at a concentration of about 0.025 millimolar (mM) to about 2 mM; the second molecule is present at a concentration of about 0.3 mM to about 125 mM; and wherein the triarylphosphine is present at a concentration of about 3 mM to about 125 mM.

17. The method of claim 14, wherein the reaction mixture further comprises a bicarbonate buffer, a borate buffer, or an acetate buffer.

18. The method of claim 14, wherein at least one of said first molecule and said second molecule comprises a dye, a fluorophore, a polymer, an affinity label, a lipid, a small molecule therapeutic agent, and a radioisotope.
